(19) United States
(12) Patent Application Publication
(10) Pub. No.: US 2019/0290633 A1
(43) Pub. Date: Sep. 26, 2019

(54) Tiotropium Inhalation Solution for Nebulization

(71) Applicant: Nephron Pharmaceuticals Corporation, West Columbia, SC (US)

(72) Inventor: Ashley Daugherty, West Columbia, SC (US)

(21) Appl. No.: 16/172,434

(22) Filed: Oct. 26, 2018

Related U.S. Application Data
(63) Continuation-in-part of application No. 16/119,209, filed on Aug. 31, 2018.
Application No. 16/119,209 is a continuation of application No. 15/796,524, filed on Oct. 27, 2017, now abandoned.

(51) Int. Cl. A61K 31/46; A61K 47/12; B65B 3/02; B65B 3/04; B65B 55/14; A61K 9/00

(52) U.S. Cl. CPC A61K 31/46; A61K 47/12; B65B 3/022; B29L 2031/712; B65B 55/14; A61K 9/0078; B65B 3/04

(57) ABSTRACT

The present invention relates to a sterile pharmaceutical composition comprising tiotropium or a pharmaceutically acceptable salt thereof, for inhalation via nebulization to a subject (e.g. a human). The invention also relates to a process for preparing the pharmaceutical composition and its use in the treatment of respiratory diseases such as chronic obstructive pulmonary disease (COPD) in a subject.

### CROSS REFERENCE TO RELATED APPLICATION

This application is a continuation-in-part of U.S. application Ser. No. 16/119,209, filed Aug. 31, 2018, which is a continuation of U.S. application Ser. No. 15/796,524, filed Oct. 27, 2017. All of the foregoing applications (hereinafter referred to as the “REFERENCE APPLICATIONS”), in their entirety, are incorporated herein by reference.

### FIELD OF THE INVENTION

The present invention relates to a sterile pharmaceutical composition comprising tiotropium or a pharmaceutically acceptable salt thereof, for inhalation via nebulization to a subject (e.g. a human). The invention also relates to a process for preparing the pharmaceutical composition and its use in the treatment of respiratory diseases such as chronic obstructive pulmonary disease (COPD) in a subject.

### BACKGROUND OF THE INVENTION

Tiotropium is a topically active anticholinergic agent indicated for the maintenance treatment of bronchospasm associated with COPD and for reducing COPD exacerbations. Several salts and hydrates of tiotropium exist, the most common salt being tiotropium bromide (CAS Registry No. 136310-93-5; designated chemically as (1α, 2β, 4β, 5α, 7β)-7-[(hydroxydi-2-thienylacetyl) oxy]-9, 9-dimethyl-3-oxa-9-azoniatricyclo [3.3.1.02, 4] nonane bromide):

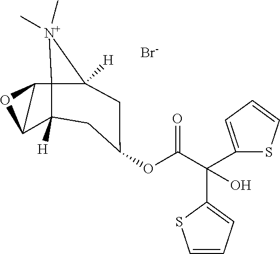

Tiotropium bromide monohydrate is commercially marketed in the United States by Boehringer Ingelheim Pharmaceuticals, Inc. under the SPIRIVA trademark:

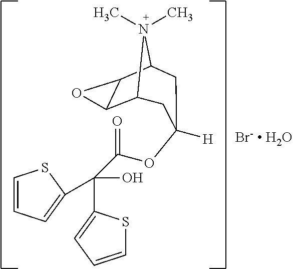

Products sold under the SPIRIVA trademark include capsules containing lactose and 18 mcg tiotropium (equivalent to 22.5 mcg tiotropium bromide monohydrate) under the approved New Drug Application No. 021395, which is hereby incorporated by reference in its entirety. The product label for SPIRIVA capsules is hereby incorporated by reference in its entirety. Boehringer Ingelheim Pharmaceuticals, Inc. also markets tiotropium bromide under the SPIRIVA RESPIMAT trademark as metered dose inhalation solutions for delivery by inhaler (1.25 mcg per inhaler actuation or 2.5 mcg tiotropium per inhaler actuation; two actuations per dose for a total delivered dose of 2.5 mcg or 5 mcg tiotropium, respectively) containing tiotropium bromide, water for injection, edetate disodium, benzalkonium chloride, and hydrochloric acid under the approved New Drug Application No. 021936, which is hereby incorporated by reference in its entirety. The product label for SPIRIVA RESPIMAT Inhalation Solution is hereby incorporated by reference in its entirety.

It is believed that a nebulizable unit dose tiotropium solution may be advantageous over metered dose inhalation formulations for several reasons. First, unit dose formulations can be delivered in sterile unit dose containers, eliminating the need for preservatives such as benzalkonium chloride which have been associated with lung irritation and paradoxical bronchoconstriction. Second, nebulizable solutions require less physical effort on the part of the patient, which may allow for improved treatment of elderly patients or other patients with limited ability to inhale. Third, a nebulization solution form may deliver more consistent dosing than the metered form. It would be of particular interest to develop edetate disodium-free formulations in order to further reduce the risk of undesirable side-effects in patients.

Experience from albuterol and ipratropium bromide systems indicated that as much as 5-10 times as much active ingredient would be required in the nebulization solution compared to the metered dose form. Unexpectedly, it is now believed that a nebulized tiotropium solution would also be more efficacious (requiring a lower dosage) than current metered dose technology. In addition, it is believed that a unit dose of tiotropium may be provided in a reduced volume for administration via nebulization, enabling the unit dose to be delivered more quickly. Accordingly, there is a need for low dose nebulizable pharmaceutical solutions that can be administered to patients in need thereof using available nebulizers.

### BRIEF SUMMARY OF THE INVENTION

Certain embodiments may provide, for example, a benzalkonium chloride-free, EDTA-free pharmaceutical composition, comprising: i) 0.0007-0.00088 wt. % tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, or 0.00088 wt. % tiotropium); ii) 0.08-0.2 wt. % sodium citrate (for example 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, or 0.2 wt. % sodium citrate); iii) 0.2-0.6 wt. % citric acid (for example 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); and iv) 0.6-0.9 wt. % sodium chloride (for example 0.6 wt. %, 0.7 wt. %, 0.8 wt. %, or 0.9 wt. % sodium chloride); and v) 97-99.2 wt. % water.

A. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free. In certain embodiments, for example, a 2 mL dose of the benzalkonium chloride-free, EDTA-free pharmaceutical composition containing 14-17.6 mcg of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, or 17.6 mcg of tiotropium) dispensed through a nebulizer may be bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the nebulized dose of the pharmaceutical composition may be bioequivalent when compared to: (a) an AUC of tiotropium of the inhaler-dispensed dose of the tiotropium solution; and (b) a Cₘₐₓ of tiotropium of the inhaler-dispensed dose of the tiotropium solution. In certain embodiments, for example, the nebulized dose of the pharmaceutical composition may provide: (a) an AUC of tiotropium in the range of 80-125% of an AUC of tiotropium of the inhaler-dispensed dose of the tiotropium solution; and (b) a Cₘₐₓ of tiotropium in the range of 80-125% of a Cₘₐₓ of tiotropium of the inhaler-dispensed dose of the tiotropium solution. In certain embodiments, for example, the nebulized dose of the pharmaceutical composition may provide: (a) an AUC of tiotropium of 25.7-40.2 pg-h/mL; and (b) a Cₘₐₓ of tiotropium of 12-19.5 pg/mL. In certain embodiments, for example, the nebulized dose of the pharmaceutical composition may be bioequivalent when compared to: an improvement in trough FEV₁ compared to placebo of the inhaler-dispensed dose of the tiotropium solution. In certain embodiments, for example, the nebulized dose may be nebulized in an air jet nebulizer.

B. In certain embodiments, for example, the concentration of the sodium citrate may be in the range of 0.1-0.11 wt. %. In certain embodiments, for example, the concentration of citric acid may be 0.4 wt. %. In certain embodiments, for example, the concentration of sodium chloride may be 0.8 wt. %. In certain embodiments, for example, the composition may be exclusive of additional excipients.

Certain embodiments may provide, for example, a pharmaceutical product comprising: i) a container having a volume in the range of 1-6 mL and; ii) a volume of a benzalkonium chloride-free, EDTA-free pharmaceutical composition (for example a sterile pharmaceutical composition) contained in the container, the pharmaceutical composition comprising: a) tiotropium; b) 0.08-0.2 wt. % sodium citrate (for example 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, or 0.2 wt. % sodium citrate); c) 0.2-0.6 wt. % citric acid (for example 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); and d) at least 97 wt. % water, the pharmaceutical product administrable by a nebulizer to provide a nebulized dose of the pharmaceutical composition that is bioequivalent to a dose of a tiotropium solution administered through an inhaler.

A. In certain embodiments, for example, the volume of the pharmaceutical composition may be 2 mL. In certain embodiments, for example, the pharmaceutical product may contain 14-17.6 mcg of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, or 17.6 mcg of tiotropium). Certain embodiments, for example, may provide a method of treating a patient, comprising administering a plurality of the pharmaceutical product according to a prescribed treatment schedule to achieve an improvement in trough FEV₁ of at least 10% or at least 100 mL above placebo. In certain embodiments, for example, the prescribed treatment schedule may extend for at least 48 weeks and the improvement may be maintained from after 1 week of the prescribed treatment until the end of the at least 48 weeks.

Certain embodiments may provide, for example, a pharmaceutical product comprising: i) a container having a volume in the range of 1-6 mL and; ii) a volume of a benzalkonium chloride-free, EDTA-free pharmaceutical composition (for example a sterile pharmaceutical composition) contained in the container, the pharmaceutical composition comprising: a) tiotropium; b) 0.08-0.2 wt. % sodium citrate (for example 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, or 0.2 wt. % sodium citrate); c) 0.2-0.6 wt. % citric acid (for example 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); and d) at least 97 wt. % water, the pharmaceutical product adapted to be dispensed with a nebulizer (for example an air-driven jet nebulizer or a vibrating mesh nebulizer) administrable by a nebulizer to provide a nebulized dose of the pharmaceutical composition that is bioequivalent to an inhaler-administered dose of a tiotropium solution.

A. In certain embodiments, for example, the volume of the pharmaceutical composition may be 2 mL. In certain embodiments, for example, the pharmaceutical product may contain 14-17.6 mcg of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, or 17.6 mcg of tiotropium). Certain embodiments, for example, may provide a method of treating a patient, comprising administering a plurality of the pharmaceutical product according to a prescribed treatment schedule to achieve an improvement in trough FEV₁ of at least 10% or at least 100 mL above placebo. In certain embodiments, for example, the prescribed treatment schedule may extend for at least 48 weeks and the improvement may be maintained from after 1 week of the prescribed treatment until the end of the at least 48 weeks.

Certain embodiments may provide, for example, a method of treating a human COPD patient, comprising administering to the patient, once daily, a unit dose of a benzalkonium chloride-free, EDTA-free pharmaceutical composition, the composition comprising: i) 0.0007-0.00088 wt. % (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, or 0.00088 wt. % tiotropium); ii) 0.08-0.2 wt. % sodium citrate (for example 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, or 0.2 wt. % sodium citrate); iii) 0.2-0.6 wt. % citric acid (for example 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-17.6 mcg of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, or 17.6 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the nebulized dose of the pharmaceutical composition may provide an improvement in trough FEV₁ in a patient that is in the range of 80-125% of an improvement in trough FEV₁ of the inhaler-dispensed dose of the tiotropium solution.

Certain embodiments may provide, for example, a benzalkonium chloride-free, EDTA-free pharmaceutical composition, comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); and iv) 0.6-0.9 wt. % sodium chloride (for example 0.6 wt. %, 0.7 wt. %, 0.8 wt. %, or 0.9 wt. % sodium chloride); and v) 97-99.2 wt. % water.

A. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

B. In certain embodiments, for example, a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, or 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer may be bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6.

C. In certain embodiments, for example, the inhaler-dispensed tiotropium solution may be administered to one or more healthy humans to provide: (a) a mean (for example a geometric or an arithmetic mean) steady-state area under the blood plasma concentration-time curve (AUC) (for example a mean (for example a geometric or an arithmetic mean) steady-state area under the blood plasma concentration-time curve from time 0 to 6 hours (AUC₀₋₆) or another specified number of hours from time 0) of tiotropium in the range of 19.5-30.5 pg-h/mL (for example an AUC (such as an AUC₀₋₆) of tiotropium in the range of 19.5-30.5 pg-h/mL with a 90% or 95% confidence interval); and (b) a mean (for example a geometric or an arithmetic mean) steady-state maximum blood plasma concentration (Cₘₐₓ) of tiotropium in the range of 9.9-15.5 pg/mL (for example a Cₘₐₓ of tiotropium in the range of 9.9-15.5 pg/mL with a 90% or 95% confidence interval). In certain embodiments, for example, the specified number of hours may be 1 hour, 2 hours, 3 hours, 4 hours, 5 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or the number of hours may be 24 hours. In certain embodiments, for example, the AUC (such as an AUC₀₋₆) may be 19.5 pg-h/mL, 19.6 pg-h/mL, 19.7 pg-h/mL, 19.8 pg-h/mL, 19.9 pg-h/mL, 20 pg-h/mL, 20.1 pg-h/mL, 20.2 pg-h/mL, 20.3 pg-h/mL, 20.4 pg-h/mL, 20.5 pg-h/mL, 20.6 pg-h/mL, 20.7 pg-h/mL, 20.8 pg-h/mL, 20.9 pg-h/mL, 21 pg-h/mL, 21.1 pg-h/mL, 21.2 pg-h/mL, 21.3 pg-h/mL, 21.4 pg-h/mL, 21.5 pg-h/mL, 21.6 pg-h/mL, 21.7 pg-h/mL, 21.8 pg-h/mL, 21.9 pg-h/mL, 22 pg-h/mL, 22.1 pg-h/mL, 22.2 pg-h/mL, 22.3 pg-h/mL, 22.4 pg-h/mL, 22.5 pg-h/mL, 22.6 pg-h/mL, 22.7 pg-h/mL, 22.8 pg-h/mL, 22.9 pg-h/mL, 23 pg-h/mL, 23.1 pg-h/mL, 23.2 pg-h/mL, 23.3 pg-h/mL, 23.4 pg-h/mL, 23.5 pg-h/mL, 23.6 pg-h/mL, 23.7 pg-h/mL, 23.8 pg-h/mL, 23.9 pg-h/mL, 24 pg-h/mL, 24.1 pg-h/mL, 24.2 pg-h/mL, 24.3 pg-h/mL, 24.4 pg-h/mL, 24.5 pg-h/mL, 24.6 pg-h/mL, 24.7 pg-h/mL, 24.8 pg-h/mL, 24.9 pg-h/mL, 25 pg-h/mL, 25.1 pg-h/mL, 25.2 pg-h/mL, 25.3 pg-h/mL, 25.4 pg-h/mL, 25.5 pg-h/mL, 25.6 pg-h/mL, 25.7 pg-h/mL, 25.8 pg-h/mL, 25.9 pg-h/mL, 26 pg-h/mL, 26.1 pg-h/mL, 26.2 pg-h/mL, 26.3 pg-h/mL, 26.4 pg-h/mL, 26.5 pg-h/mL, 26.6 pg-h/mL, 26.7 pg-h/mL, 26.8 pg-h/mL, 26.9 pg-h/mL, 27 pg-h/mL, 27.1 pg-h/mL, 27.2 pg-h/mL, 27.3 pg-h/mL, 27.4 pg-h/mL, 27.5 pg-h/mL, 27.6 pg-h/mL, 27.7 pg-h/mL, 27.8 pg-h/mL, 27.9 pg-h/mL, 28 pg-h/mL, 28.1 pg-h/mL, 28.2 pg-h/mL, 28.3 pg-h/mL, 28.4 pg-h/mL, 28.5 pg-h/mL, 28.6 pg-h/mL, 28.7 pg-h/mL, 28.8 pg-h/mL, 28.9 pg-h/mL, 29 pg-h/mL, 29.1 pg-h/mL, 29.2 pg-h/mL, 29.3 pg-h/mL, 29.4 pg-h/mL, 29.5 pg-h/mL, 29.6 pg-h/mL, 29.7 pg-h/mL, 29.8 pg-h/mL, 29.9 pg-h/mL, 30 pg-h/mL, 30.1 pg-h/mL, 30.2 pg-h/mL, 30.3 pg-h/mL, 30.4 pg-h/mL, or the AUC (such as an AUC₀₋₆) may be 30.5 pg-h/mL. In certain embodiments, for example, the Cₘₐₓ may be 9.9 pg/mL, 10 pg/mL, 10.1 pg/mL, 10.2 pg/mL, 10.3 pg/mL, 10.4 pg/mL, 10.5 pg/mL, 10.6 pg/mL, 10.7 pg/mL, 10.8 pg/mL, 10.9 pg/mL, 11 pg/mL, 11.1 pg/mL, 11.2 pg/mL, 11.3 pg/mL, 11.4 pg/mL, 11.5 pg/mL, 11.6 pg/mL, 11.7 pg/mL, 11.8 pg/mL, 11.9 pg/mL, 12 pg/mL, 12.1 pg/mL, 12.2 pg/mL, 12.3 pg/mL, 12.4 pg/mL, 12.5 pg/mL, 12.6 pg/mL, 12.7 pg/mL, 12.8 pg/mL, 12.9 pg/mL, 13 pg/mL, 13.1 pg/mL, 13.2 pg/mL, 13.3 pg/mL, 13.4 pg/mL, 13.5 pg/mL, 13.6 pg/mL, 13.7 pg/mL, 13.8 pg/mL, 13.9 pg/mL, 14 pg/mL, 14.1 pg/mL, 14.2 pg/mL, 14.3 pg/mL, 14.4 pg/mL, 14.5 pg/mL, 14.6 pg/mL, 14.7 pg/mL, 14.8 pg/mL, 14.9 pg/mL, 15 pg/mL, 15.1 pg/mL, 15.2 pg/mL, 15.3 pg/mL, 15.4 pg/mL, or the Cₘₐₓ may be 15.5 pg/mL.

D. In certain embodiments, for example, the inhaler-dispensed tiotropium solution may be administered to one or more healthy humans to provide: (a) an AUC (such as an AUC₀₋₆) of tiotropium in the range of 25.7-40.2 pg-h/mL (for example an AUC (such as an AUC₀₋₆) of tiotropium in the range of 25.7-40.2 pg-h/mL with a 90% or 95% confidence interval); and (b) a Cₘₐₓ of tiotropium in the range of 12-19.5 pg/mL (for example a Cₘₐₓ of tiotropium in the range of 12-19.5 pg/mL with a 90% or 95% confidence interval). In certain embodiments, for example, the AUC (such as an AUC₀₋₆) may be 25.7 pg-h/mL, 25.8 pg-h/mL, 25.9 pg-h/mL, 26 pg-h/mL, 26.1 pg-h/mL, 26.2 pg-h/mL, 26.3 pg-h/mL, 26.4 pg-h/mL, 26.5 pg-h/mL, 26.6 pg-h/mL, 26.7 pg-h/mL, 26.8 pg-h/mL, 26.9 pg-h/mL, 27 pg-h/mL, 27.1 pg-h/mL, 27.2 pg-h/mL, 27.3 pg-h/mL, 27.4 pg-h/mL, 27.5 pg-h/mL, 27.6 pg-h/mL, 27.7 pg-h/mL, 27.8 pg-h/mL, 27.9 pg-h/mL, 28 pg-h/mL, 28.1 pg-h/mL, 28.2 pg-h/mL, 28.3 pg-h/mL, 28.4 pg-h/mL, 28.5 pg-h/mL, 28.6 pg-h/mL, 28.7 pg-h/mL, 28.8 pg-h/mL, 28.9 pg-h/mL, 29 pg-h/mL, 29.1 pg-h/mL, 29.2 pg-h/mL, 29.3 pg-h/mL, 29.4 pg-h/mL, 29.5 pg-h/mL, 29.6 pg-h/mL, 29.7 pg-h/mL, 29.8 pg-h/mL, 29.9 pg-h/mL, 30 pg-h/mL, 30.1 pg-h/mL, 30.2 pg-h/mL, 30.3 pg-h/mL, 30.4 pg-h/mL, 30.5 pg-h/mL, 30.6 pg-h/mL, 30.7 pg-h/mL, 30.8 pg-h/mL, 30.9 pg-h/mL, 31 pg-h/mL, 31.1 pg-h/mL, 31.2 pg-h/mL, 31.3 pg-h/mL, 31.4 pg-h/mL, 31.5 pg-h/mL, 31.6 pg-h/mL, 31.7 pg-h/mL, 31.8 pg-h/mL, 31.9 pg-h/mL, 32 pg-h/mL, 32.1 pg-h/mL, 32.2 pg-h/mL, 32.3 pg-h/mL, 32.4 pg-h/mL, 32.5 pg-h/mL, 32.6 pg-h/mL, 32.7 pg-h/mL, 32.8 pg-h/mL, 32.9 pg-h/mL, 33 pg-h/mL, 33.1 pg-h/mL, 33.2 pg-h/mL, 33.3 pg-h/mL, 33.4 pg-h/mL, 33.5 pg-h/mL, 33.6 pg-h/mL, 33.7 pg-h/mL, 33.8 pg-h/mL, 33.9 pg-h/mL, 34 pg-h/mL, 34.1 pg-h/mL, 34.2 pg-h/mL, 34.3 pg-h/mL, 34.4 pg-h/mL, 34.5 pg-h/mL, 34.6 pg-h/mL, 34.7 pg-h/mL, 34.8 pg-h/mL, 34.9 pg-h/mL, 35 pg-h/mL, 35.1 pg-h/mL, 35.2 pg-h/mL, 35.3 pg-h/mL, 35.4 pg-h/mL, 35.5 pg-h/mL, 35.6 pg-h/mL, 35.7 pg-h/mL, 35.8 pg-h/mL, 35.9 pg-h/mL, 36 pg-h/mL, 36.1 pg-h/mL, 36.2 pg-h/mL, 36.3 pg-h/mL, 36.4 pg-h/mL, 36.5 pg-h/mL, 36.6 pg-h/mL, 36.7 pg-h/mL, 36.8 pg-h/mL, 36.9 pg-h/mL, 37 pg-h/mL, 37.1 pg-h/mL, 37.2 pg-h/mL, 37.3 pg-h/mL, 37.4 pg-h/mL, 37.5 pg-h/mL, 37.6 pg-h/mL, 37.7 pg-h/mL, 37.8 pg-h/mL, 37.9 pg-h/mL, 38 pg-h/mL, 38.1 pg-h/mL, 38.2 pg-h/mL, 38.3 pg-h/mL, 38.4 pg-h/mL, 38.5 pg-h/mL, 38.6 pg-h/mL, 38.7 pg-h/mL, 38.8 pg-h/mL, 38.9 pg-h/mL, 39 pg-h/mL, 39.1 pg-h/mL, 39.2 pg-h/mL, 39.3 pg-h/mL, 39.4 pg-h/mL, 39.5 pg-h/mL, 39.6 pg-h/mL, 39.7 pg-h/mL, 39.8 pg-h/mL, 39.9 pg-h/mL, 40 pg-h/mL, 40.1 pg-h/mL, or the AUC (such as an AUC₀₋₆) may be 40.2 pg-h/mL. In certain embodiments, for example, the Cₘₐₓ may be 12 pg/mL, 12.1 pg/mL, 12.2 pg/mL, 12.3 pg/mL, 12.4 pg/mL, 12.5 pg/mL, 12.6 pg/mL, 12.7 pg/mL, 12.8 pg/mL, 12.9 pg/mL, 13 pg/mL, 13.1 pg/mL, 13.2 pg/mL, 13.3 pg/mL, 13.4 pg/mL, 13.5 pg/mL, 13.6 pg/mL, 13.7 pg/mL, 13.8 pg/mL, 13.9 pg/mL, 14 pg/mL, 14.1 pg/mL, 14.2 pg/mL, 14.3 pg/mL, 14.4 pg/mL, 14.5 pg/mL, 14.6 pg/mL, 14.7 pg/mL, 14.8 pg/mL, 14.9 pg/mL, 15 pg/mL, 15.1 pg/mL, 15.2 pg/mL, 15.3 pg/mL, 15.4 pg/mL, 15.5 pg/mL, 15.6 pg/mL, 15.7 pg/mL, 15.8 pg/mL, 15.9 pg/mL, 16 pg/mL, 16.1 pg/mL, 16.2 pg/mL, 16.3 pg/mL, 16.4 pg/mL, 16.5 pg/mL, 16.6 pg/mL, 16.7 pg/mL, 16.8 pg/mL, 16.9 pg/mL, 17 pg/mL, 17.1 pg/mL, 17.2 pg/mL, 17.3 pg/mL, 17.4 pg/mL, 17.5 pg/mL, 17.6 pg/mL, 17.7 pg/mL, 17.8 pg/mL, 17.9 pg/mL, 18 pg/mL, 18.1 pg/mL, 18.2 pg/mL, 18.3 pg/mL, 18.4 pg/mL, 18.5 pg/mL, 18.6 pg/mL, 18.7 pg/mL, 18.8 pg/mL, 18.9 pg/mL, 19 pg/mL, 19.1 pg/mL, 19.2 pg/mL, 19.3 pg/mL, 19.4 pg/mL, or the Cₘₐₓ may be 19.5 pg/mL.

E. In certain embodiments, for example, a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer may be administered to one or more healthy humans to provide: (a) an AUC (such as an AUC₀₋₆) of tiotropium in the range of 19.5-30.5 pg-h/mL (for example an AUC (such as an AUC₀₋₆) of tiotropium in the range of 19.5-30.5 pg-h/mL with a 90% or 95% confidence interval); and (b) a Cₘₐₓ of tiotropium in the range of 9.9-15.5 pg/mL (for example a Cₘₐₓ of tiotropium in the range of 9.9-15.5 pg/mL with a 90% or 95% confidence interval). In certain embodiments, for example, the AUC (such as an AUC₀₋₆) may be 19.5 pg-h/mL, 19.6 pg-h/mL, 19.7 pg-h/mL, 19.8 pg-h/mL, 19.9 pg-h/mL, 20 pg-h/mL, 20.1 pg-h/mL, 20.2 pg-h/mL, 20.3 pg-h/mL, 20.4 pg-h/mL, 20.5 pg-h/mL, 20.6 pg-h/mL, 20.7 pg-h/mL, 20.8 pg-h/mL, 20.9 pg-h/mL, 21 pg-h/mL, 21.1 pg-h/mL, 21.2 pg-h/mL, 21.3 pg-h/mL, 21.4 pg-h/mL, 21.5 pg-h/mL, 21.6 pg-h/mL, 21.7 pg-h/mL, 21.8 pg-h/mL, 21.9 pg-h/mL, 22 pg-h/mL, 22.1 pg-h/mL, 22.2 pg-h/mL, 22.3 pg-h/mL, 22.4 pg-h/mL, 22.5 pg-h/mL, 22.6 pg-h/mL, 22.7 pg-h/mL, 22.8 pg-h/mL, 22.9 pg-h/mL, 23 pg-h/mL, 23.1 pg-h/mL, 23.2 pg-h/mL, 23.3 pg-h/mL, 23.4 pg-h/mL, 23.5 pg-h/mL, 23.6 pg-h/mL, 23.7 pg-h/mL, 23.8 pg-h/mL, 23.9 pg-h/mL, 24 pg-h/mL, 24.1 pg-h/mL, 24.2 pg-h/mL, 24.3 pg-h/mL, 24.4 pg-h/mL, 24.5 pg-h/mL, 24.6 pg-h/mL, 24.7 pg-h/mL, 24.8 pg-h/mL, 24.9 pg-h/mL, 25 pg-h/mL, 25.1 pg-h/mL, 25.2 pg-h/mL, 25.3 pg-h/mL, 25.4 pg-h/mL, 25.5 pg-h/mL, 25.6 pg-h/mL, 25.7 pg-h/mL, 25.8 pg-h/mL, 25.9 pg-h/mL, 26 pg-h/mL, 26.1 pg-h/mL, 26.2 pg-h/mL, 26.3 pg-h/mL, 26.4 pg-h/mL, 26.5 pg-h/mL, 26.6 pg-h/mL, 26.7 pg-h/mL, 26.8 pg-h/mL, 26.9 pg-h/mL, 27 pg-h/mL, 27.1 pg-h/mL, 27.2 pg-h/mL, 27.3 pg-h/mL, 27.4 pg-h/mL, 27.5 pg-h/mL, 27.6 pg-h/mL, 27.7 pg-h/mL, 27.8 pg-h/mL, 27.9 pg-h/mL, 28 pg-h/mL, 28.1 pg-h/mL, 28.2 pg-h/mL, 28.3 pg-h/mL, 28.4 pg-h/mL, 28.5 pg-h/mL, 28.6 pg-h/mL, 28.7 pg-h/mL, 28.8 pg-h/mL, 28.9 pg-h/mL, 29 pg-h/mL, 29.1 pg-h/mL, 29.2 pg-h/mL, 29.3 pg-h/mL, 29.4 pg-h/mL, 29.5 pg-h/mL, 29.6 pg-h/mL, 29.7 pg-h/mL, 29.8 pg-h/mL, 29.9 pg-h/mL, 30 pg-h/mL, 30.1 pg-h/mL, 30.2 pg-h/mL, 30.3 pg-h/mL, 30.4 pg-h/mL, or the AUC (such as an AUC₀₋₆) may be 30.5 pg-h/mL. In certain embodiments, for example, the Cₘₐₓ may be 9.9 pg/mL, 10 pg/mL, 10.1 pg/mL, 10.2 pg/mL, 10.3 pg/mL, 10.4 pg/mL, 10.5 pg/mL, 10.6 pg/mL, 10.7 pg/mL, 10.8 pg/mL, 10.9 pg/mL, 11 pg/mL, 11.1 pg/mL, 11.2 pg/mL, 11.3 pg/mL, 11.4 pg/mL, 11.5 pg/mL, 11.6 pg/mL, 11.7 pg/mL, 11.8 pg/mL, 11.9 pg/mL, 12 pg/mL, 12.1 pg/mL, 12.2 pg/mL, 12.3 pg/mL, 12.4 pg/mL, 12.5 pg/mL, 12.6 pg/mL, 12.7 pg/mL, 12.8 pg/mL, 12.9 pg/mL, 13 pg/mL, 13.1 pg/mL, 13.2 pg/mL, 13.3 pg/mL, 13.4 pg/mL, 13.5 pg/mL, 13.6 pg/mL, 13.7 pg/mL, 13.8 pg/mL, 13.9 pg/mL, 14 pg/mL, 14.1 pg/mL, 14.2 pg/mL, 14.3 pg/mL, 14.4 pg/mL, 14.5 pg/mL, 14.6 pg/mL, 14.7 pg/mL, 14.8 pg/mL, 14.9 pg/mL, 15 pg/mL, 15.1 pg/mL, 15.2 pg/mL, 15.3 pg/mL, 15.4 pg/mL, or the Cₘₐₓ may be 15.5 pg/mL.

F. In certain embodiments, for example, a 2 mL nebulized dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer may be administered to one or more healthy humans to provide: (a) an AUC (such as an AUC₀₋₆) of tiotropium in the range of 25.7-40.2 pg-h/mL (for example an AUC (such as an AUC₀₋₆) of tiotropium in the range of 25.7-40.2 pg-h/mL with a 90% or 95% confidence interval); and (b) a Cₘₐₓ of tiotropium in the range of 12-19.5 pg/mL (for example a Cₘₐₓ of tiotropium in the range of 12-19.5 pg/mL with a 90% or 95% confidence interval). In certain embodiments, for example, the AUC (such as an AUC₀₋₆) may be 25.7 pg-h/mL, 25.8 pg-h/mL, 25.9 pg-h/mL, 26 pg-h/mL, 26.1 pg-h/mL, 26.2 pg-h/mL, 26.3 pg-h/mL, 26.4 pg-h/mL, 26.5 pg-h/mL, 26.6 pg-h/mL, 26.7 pg-h/mL, 26.8 pg-h/mL, 26.9 pg-h/mL, 27 pg-h/mL, 27.1 pg-h/mL, 27.2 pg-h/mL, 27.3 pg-h/mL, 27.4 pg-h/mL, 27.5 pg-h/mL, 27.6 pg-h/mL, 27.7 pg-h/mL, 27.8 pg-h/mL, 27.9 pg-h/mL, 28 pg-h/mL, 28.1 pg-h/mL, 28.2 pg-h/mL, 28.3 pg-h/mL, 28.4 pg-h/mL, 28.5 pg-h/mL, 28.6 pg-h/mL, 28.7 pg-h/mL, 28.8 pg-h/mL, 28.9 pg-h/mL, 29 pg-h/mL, 29.1 pg-h/mL, 29.2 pg-h/mL, 29.3 pg-h/mL, 29.4 pg-h/mL, 29.5 pg-h/mL, 29.6 pg-h/mL, 29.7 pg-h/mL, 29.8 pg-h/mL, 29.9 pg-h/mL, 30 pg-h/mL, 30.1 pg-h/mL, 30.2 pg-h/mL, 30.3 pg-h/mL, 30.4 pg-h/mL, 30.5 pg-h/mL, 30.6 pg-h/mL, 30.7 pg-h/mL, 30.8 pg-h/mL, 30.9 pg-h/mL, 31 pg-h/mL, 31.1 pg-h/mL, 31.2 pg-h/mL, 31.3 pg-h/mL, 31.4 pg-h/mL, 31.5 pg-h/mL, 31.6 pg-h/mL, 31.7 pg-h/mL, 31.8 pg-h/mL, 31.9 pg-h/mL, 32 pg-h/mL, 32.1 pg-h/mL, 32.2 pg-h/mL, 32.3 pg-h/mL, 32.4 pg-h/mL, 32.5 pg-h/mL, 32.6 pg-h/mL, 32.7 pg-h/mL, 32.8 pg-h/mL, 32.9 pg-h/mL, 33 pg-h/mL, 33.1 pg-h/mL, 33.2 pg-h/mL, 33.3 pg-h/mL, 33.4 pg-h/mL, 33.5 pg-h/mL, 33.6 pg-h/mL, 33.7 pg-h/mL, 33.8 pg-h/mL, 33.9 pg-h/mL, 34 pg-h/mL, 34.1 pg-h/mL, 34.2 pg-h/mL, 34.3 pg-h/mL, 34.4 pg-h/mL, 34.5 pg-h/mL, 34.6 pg-h/mL, 34.7 pg-h/mL, 34.8 pg-h/mL, 34.9 pg-h/mL, 35 pg-h/mL, 35.1 pg-h/mL, 35.2 pg-h/mL, 35.3 pg-h/mL, 35.4 pg-h/mL, 35.5 pg-h/mL, 35.6 pg-h/mL, 35.7 pg-h/mL, 35.8 pg-h/mL, 35.9 pg-h/mL, 36 pg-h/mL, 36.1 pg-h/mL, 36.2 pg-h/mL, 36.3 pg-h/mL, 36.4 pg-h/mL, 36.5 pg-h/mL, 36.6 pg-h/mL, 36.7 pg-h/mL, 36.8 pg-h/mL, 36.9 pg-h/mL, 37 pg-h/mL, 37.1 pg-h/mL, 37.2 pg-h/mL, 37.3 pg-h/mL, 37.4 pg-h/mL, 37.5 pg-h/mL, 37.6 pg-h/mL, 37.7 pg-h/mL, 37.8 pg-h/mL, 37.9 pg-h/mL, 38 pg-h/mL, 38.1 pg-h/mL, 38.2 pg-h/mL, 38.3 pg-h/mL, 38.4 pg-h/mL, 38.5 pg-h/mL, 38.6 pg-h/mL, 38.7 pg-h/mL, 38.8 pg-h/mL, 38.9 pg-h/mL, 39 pg-h/mL, 39.1 pg-h/mL, 39.2 pg-h/mL, 39.3 pg-h/mL, 39.4 pg-h/mL, 39.5 pg-h/mL, 39.6 pg-h/mL, 39.7 pg-h/mL, 39.8 pg-h/mL, 39.9 pg-h/mL, 40 pg-h/mL, 40.1 pg-h/mL, or the AUC (such as an AUC₀₋₆) may be 40.2 pg-h/mL. In certain embodiments, for example, the Cₘₐₓ may be 12 pg/mL, 12.1 pg/mL, 12.2 pg/mL, 12.3 pg/mL, 12.4 pg/mL, 12.5 pg/mL, 12.6 pg/mL, 12.7 pg/mL, 12.8 pg/mL, 12.9 pg/mL, 13 pg/mL, 13.1 pg/mL, 13.2 pg/mL, 13.3 pg/mL, 13.4 pg/mL, 13.5 pg/mL, 13.6 pg/mL, 13.7 pg/mL, 13.8 pg/mL, 13.9 pg/mL, 14 pg/mL, 14.1 pg/mL, 14.2 pg/mL, 14.3 pg/mL, 14.4 pg/mL, 14.5 pg/mL, 14.6 pg/mL, 14.7 pg/mL, 14.8 pg/mL, 14.9 pg/mL, 15 pg/mL, 15.1 pg/mL, 15.2 pg/mL, 15.3 pg/mL, 15.4 pg/mL, 15.5 pg/mL, 15.6 pg/mL, 15.7 pg/mL, 15.8 pg/mL, 15.9 pg/mL, 16 pg/mL, 16.1 pg/mL, 16.2 pg/mL, 16.3 pg/mL, 16.4 pg/mL, 16.5 pg/mL, 16.6 pg/mL, 16.7 pg/mL, 16.8 pg/mL, 16.9 pg/mL, 17 pg/mL, 17.1 pg/mL, 17.2 pg/mL, 17.3 pg/mL, 17.4 pg/mL, 17.5 pg/mL, 17.6 pg/mL, 17.7 pg/mL, 17.8 pg/mL, 17.9 pg/mL, 18 pg/mL, 18.1 pg/mL, 18.2 pg/mL, 18.3 pg/mL, 18.4 pg/mL, 18.5 pg/mL, 18.6 pg/mL, 18.7 pg/mL, 18.8 pg/mL, 18.9 pg/mL, 19 pg/mL, 19.1 pg/mL, 19.2 pg/mL, 19.3 pg/mL, 19.4 pg/mL, or the Cₘₐₓ may be 19.5 pg/mL.

G. In certain embodiments, for example, the tiotropium may be present in the pharmaceutical composition at a concentration in the range of 7-8.8 mcg/mL or 7.8-10 mcg/mL (for example the concentration of the tiotropium present in the pharmaceutical composition may be 7 mcg/mL, 7.1 mcg/mL, 7.2 mcg/mL, 7.3 mcg/mL, 7.4 mcg/mL, 7.5 mcg/mL, 7.6 mcg/mL, 7.7 mcg/mL, 7.8 mcg/mL, 7.9 mcg/mL, 8 mcg/mL, 8.1 mcg/mL, 8.2 mcg/mL, 8.3 mcg/mL, 8.4 mcg/mL, 8.5 mcg/mL, 8.6 mcg/mL, 8.7 mcg/mL, 8.8 mcg/mL, 8.9 mcg/mL, 9 mcg/mL, 9.1 mcg/mL, 9.2 mcg/mL, 9.3 mcg/mL, 9.4 mcg/mL, 9.5 mcg/mL, 9.6 mcg/mL, 9.7 mcg/mL, 9.8 mcg/mL, 9.9 mcg/mL, or the concentration of the tiotropium present in the pharmaceutical composition may be 10 mcg/mL).

Certain embodiments may provide, for example, a benzalkonium chloride-free, EDTA-free pharmaceutical composition, comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water.

A. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

B. In certain embodiments, for example, a 2 mL nebulized dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer may be bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6.

C. In certain embodiments, for example, the inhaler-dispensed tiotropium solution may be administered to one or more healthy humans to provide: (a) an AUC (such as an AUC₀₋₆) of tiotropium in the range of 9.7-15.3 pg-h/mL (for example an AUC (such as an AUC₀₋₆) of tiotropium in the range of 9.7-15.3 pg-h/mL with a 90% or 95% confidence interval); and (b) a Cₘₐₓ of tiotropium in the range of 4.9-7.8 pg/mL (for example a Cₘₐₓ of tiotropium in the range of 4.9-7.8 pg/mL with a 90% or 95% confidence interval). In certain embodiments, for example, the AUC (such as an AUC₀₋₆) may be 9.7 pg-h/mL, 9.8 pg-h/mL, 9.9 pg-h/mL, 10 pg-h/mL, 10.1 pg-h/mL, 10.2 pg-h/mL, 10.3 pg-h/mL, 10.4 pg-h/mL, 10.5 pg-h/mL, 10.6 pg-h/mL, 10.7 pg-h/mL, 10.8 pg-h/mL, 10.9 pg-h/mL, 11 pg-h/mL, 11.1 pg-h/mL, 11.2 pg-h/mL, 11.3 pg-h/mL, 11.4 pg-h/mL, 11.5 pg-h/mL, 11.6 pg-h/mL, 11.7 pg-h/mL, 11.8 pg-h/mL, 11.9 pg-h/mL, 12 pg-h/mL, 12.1 pg-h/mL, 12.2 pg-h/mL, 12.3 pg-h/mL, 12.4 pg-h/mL, 12.5 pg-h/mL, 12.6 pg-h/mL, 12.7 pg-h/mL, 12.8 pg-h/mL, 12.9 pg-h/mL, 13 pg-h/mL, 13.1 pg-h/mL, 13.2 pg-h/mL, 13.3 pg-h/mL, 13.4 pg-h/mL, 13.5 pg-h/mL, 13.6 pg-h/mL, 13.7 pg-h/mL, 13.8 pg-h/mL, 13.9 pg-h/mL, 14 pg-h/mL, 14.1 pg-h/mL, 14.2 pg-h/mL, 14.3 pg-h/mL, 14.4 pg-h/mL, 14.5 pg-h/mL, 14.6 pg-h/mL, 14.7 pg-h/mL, 14.8 pg-h/mL, 14.9 pg-h/mL, 15 pg-h/mL, 15.1 pg-h/mL, 15.2 pg-h/mL, or the AUC (such as an AUC₀₋₆) may be 15.3 pg-h/mL. In certain embodiments, for example, the Cₘₐₓ may be 4.9 pg/mL, 5 pg/mL, 5.1 pg/mL, 5.2 pg/mL, 5.3 pg/mL, 5.4 pg/mL, 5.5 pg/mL, 5.6 pg/mL, 5.7 pg/mL, 5.8 pg/mL, 5.9 pg/mL, 6 pg/mL, 6.1 pg/mL, 6.2 pg/mL, 6.3 pg/mL, 6.4 pg/mL, 6.5 pg/mL, 6.6 pg/mL, 6.7 pg/mL, 6.8 pg/mL, 6.9 pg/mL, 7 pg/mL, 7.1 pg/mL, 7.2 pg/mL, 7.3 pg/mL, 7.4 pg/mL, 7.5 pg/mL, 7.6 pg/mL, 7.7 pg/mL, or the Cₘₐₓ may be 7.8 pg/mL.

D. In certain embodiments, for example, the inhaler-dispensed tiotropium solution may be administered to one or more healthy humans to provide: (a) an AUC (such as an AUC₀₋₆) of tiotropium in the range of 12.8-20.1 pg-h/mL (for example an AUC (such as an AUC₀₋₆) of tiotropium in the range of 12.8-20.1 pg-h/mL with a 90% or 95% confidence interval); and (b) a Cₘₐₓ of tiotropium in the range of 6-9.8 pg/mL (for example a Cₘₐₓ of tiotropium in the range of 6-9.8 pg/mL with a 90% or 95% confidence interval). In certain embodiments, for example, the AUC (such as an AUC₀₋₆) may be 12.8 pg-h/mL, 12.9 pg-h/mL, 13 pg-h/mL, 13.1 pg-h/mL, 13.2 pg-h/mL, 13.3 pg-h/mL, 13.4 pg-h/mL, 13.5 pg-h/mL, 13.6 pg-h/mL, 13.7 pg-h/mL, 13.8 pg-h/mL, 13.9 pg-h/mL, 14 pg-h/mL, 14.1 pg-h/mL, 14.2 pg-h/mL, 14.3 pg-h/mL, 14.4 pg-h/mL, 14.5 pg-h/mL, 14.6 pg-h/mL, 14.7 pg-h/mL, 14.8 pg-h/mL, 14.9 pg-h/mL, 15 pg-h/mL, 15.1 pg-h/mL, 15.2 pg-h/mL, 15.3 pg-h/mL, 15.4 pg-h/mL, 15.5 pg-h/mL, 15.6 pg-h/mL, 15.7 pg-h/mL, 15.8 pg-h/mL, 15.9 pg-h/mL, 16 pg-h/mL, 16.1 pg-h/mL, 16.2 pg-h/mL, 16.3 pg-h/mL, 16.4 pg-h/mL, 16.5 pg-h/mL, 16.6 pg-h/mL, 16.7 pg-h/mL, 16.8 pg-h/mL, 16.9 pg-h/mL, 17 pg-h/mL, 17.1 pg-h/mL, 17.2 pg-h/mL, 17.3 pg-h/mL, 17.4 pg-h/mL, 17.5 pg-h/mL, 17.6 pg-h/mL, 17.7 pg-h/mL, 17.8 pg-h/mL, 17.9 pg-h/mL, 18 pg-h/mL, 18.1 pg-h/mL, 18.2 pg-h/mL, 18.3 pg-h/mL, 18.4 pg-h/mL, 18.5 pg-h/mL, 18.6 pg-h/mL, 18.7 pg-h/mL, 18.8 pg-h/mL, 18.9 pg-h/mL, 19 pg-h/mL, 19.1 pg-h/mL, 19.2 pg-h/mL, 19.3 pg-h/mL, 19.4 pg-h/mL, 19.5 pg-h/mL, 19.6 pg-h/mL, 19.7 pg-h/mL, 19.8 pg-h/mL, 19.9 pg-h/mL, 20 pg-h/mL, or the AUC (such as an AUC₀₋₆) may be 20.1 pg-h/mL. In certain embodiments, for example, the Cₘₐₓ may be 6 pg/mL, 6.1 pg/mL, 6.2 pg/mL, 6.3 pg/mL, 6.4 pg/mL, 6.5 pg/mL, 6.6 pg/mL, 6.7 pg/mL, 6.8 pg/mL, 6.9 pg/mL, 7 pg/mL, 7.1 pg/mL, 7.2 pg/mL, 7.3 pg/mL, 7.4 pg/mL, 7.5 pg/mL, 7.6 pg/mL, 7.7 pg/mL, 7.8 pg/mL, 7.9 pg/mL, 8 pg/mL, 8.1 pg/mL, 8.2 pg/mL, 8.3 pg/mL, 8.4 pg/mL, 8.5 pg/mL, 8.6 pg/mL, 8.7 pg/mL, 8.8 pg/mL, 8.9 pg/mL, 9 pg/mL, 9.1 pg/mL, 9.2 pg/mL, 9.3 pg/mL, 9.4 pg/mL, 9.5 pg/mL, 9.6 pg/mL, 9.7 pg/mL, 9.8 pg/mL, or the Cₘₐₓ may be 9.8 pg/mL.

E. In certain embodiments, for example, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer may be administered to one or more healthy human to provide: (a) an AUC (such as an AUC₀₋₆) of tiotropium in the range of 9.7-15.3 pg-h/mL (for example an AUC (such as an AUC₀₋₆) of tiotropium in the range of 9.7-15.3 pg-h/mL with a 90% or 95% confidence interval); and (b) a Cₘₐₓ of tiotropium in the range of 4.9-7.8 pg/mL (for example a Cₘₐₓ of tiotropium in the range of 4.9-7.8 pg/mL with a 90% or 95% confidence interval). In certain embodiments, for example, the AUC (such as an AUC₀₋₆) may be 9.7 pg-h/mL, 9.8 pg-h/mL, 9.9 pg-h/mL, 10 pg-h/mL, 10.1 pg-h/mL, 10.2 pg-h/mL, 10.3 pg-h/mL, 10.4 pg-h/mL, 10.5 pg-h/mL, 10.6 pg-h/mL, 10.7 pg-h/mL, 10.8 pg-h/mL, 10.9 pg-h/mL, 11 pg-h/mL, 11.1 pg-h/mL, 11.2 pg-h/mL, 11.3 pg-h/mL, 11.4 pg-h/mL, 11.5 pg-h/mL, 11.6 pg-h/mL, 11.7 pg-h/mL, 11.8 pg-h/mL, 11.9 pg-h/mL, 12 pg-h/mL, 12.1 pg-h/mL, 12.2 pg-h/mL, 12.3 pg-h/mL, 12.4 pg-h/mL, 12.5 pg-h/mL, 12.6 pg-h/mL, 12.7 pg-h/mL, 12.8 pg-h/mL, 12.9 pg-h/mL, 13 pg-h/mL, 13.1 pg-h/mL, 13.2 pg-h/mL, 13.3 pg-h/mL, 13.4 pg-h/mL, 13.5 pg-h/mL, 13.6 pg-h/mL, 13.7 pg-h/mL, 13.8 pg-h/mL, 13.9 pg-h/mL, 14 pg-h/mL, 14.1 pg-h/mL, 14.2 pg-h/mL, 14.3 pg-h/mL, 14.4 pg-h/mL, 14.5 pg-h/mL, 14.6 pg-h/mL, 14.7 pg-h/mL, 14.8 pg-h/mL, 14.9 pg-h/mL, 15 pg-h/mL, 15.1 pg-h/mL, 15.2 pg-h/mL, or the AUC (such as an AUC₀₋₆) may be 15.3 pg-h/mL. In certain embodiments, for example, the Cₘₐₓ may be 4.9 pg/mL, 5 pg/mL, 5.1 pg/mL, 5.2 pg/mL, 5.3 pg/mL, 5.4 pg/mL, 5.5 pg/mL, 5.6 pg/mL, 5.7 pg/mL, 5.8 pg/mL, 5.9 pg/mL, 6 pg/mL, 6.1 pg/mL, 6.2 pg/mL, 6.3 pg/mL, 6.4 pg/mL, 6.5 pg/mL, 6.6 pg/mL, 6.7 pg/mL, 6.8 pg/mL, 6.9 pg/mL, 7 pg/mL, 7.1 pg/mL, 7.2 pg/mL, 7.3 pg/mL, 7.4 pg/mL, 7.5 pg/mL, 7.6 pg/mL, 7.7 pg/mL, or the Cₘₐₓ may be 7.8 pg/mL.

F. In certain embodiments, for example, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer may be administered to one or more healthy human to provide: (a) an AUC (such as an AUC₀₋₆) of tiotropium in the range of 12.8-20.1 pg-h/mL (for example an AUC (such as an AUC₀₋₆) of tiotropium in the range of 12.8-20.1 with a 90% or 95% confidence interval); and (b) a Cₘₐₓ of tiotropium in the range of 6-9.8 pg/mL (for example a Cₘₐₓ of tiotropium in the range of 6-9.8 pg/mL a 90% or 95% confidence interval). In certain embodiments, for example, the AUC (such as an AUC₀₋₆) may be 12.8 pg-h/mL, 12.9 pg-h/mL, 13 pg-h/mL, 13.1 pg-h/mL, 13.2 pg-h/mL, 13.3 pg-h/mL, 13.4 pg-h/mL, 13.5 pg-h/mL, 13.6 pg-h/mL, 13.7 pg-h/mL, 13.8 pg-h/mL, 13.9 pg-h/mL, 14 pg-h/mL, 14.1 pg-h/mL, 14.2 pg-h/mL, 14.3 pg-h/mL, 14.4 pg-h/mL, 14.5 pg-h/mL, 14.6 pg-h/mL, 14.7 pg-h/mL, 14.8 pg-h/mL, 14.9 pg-h/mL, 15 pg-h/mL, 15.1 pg-h/mL, 15.2 pg-h/mL, 15.3 pg-h/mL, 15.4 pg-h/mL, 15.5 pg-h/mL, 15.6 pg-h/mL, 15.7 pg-h/mL, 15.8 pg-h/mL, 15.9 pg-h/mL, 16 pg-h/mL, 16.1 pg-h/mL, 16.2 pg-h/mL, 16.3 pg-h/mL, 16.4 pg-h/mL, 16.5 pg-h/mL, 16.6 pg-h/mL, 16.7 pg-h/mL, 16.8 pg-h/mL, 16.9 pg-h/mL, 17 pg-h/mL, 17.1 pg-h/mL, 17.2 pg-h/mL, 17.3 pg-h/mL, 17.4 pg-h/mL, 17.5 pg-h/mL, 17.6 pg-h/mL, 17.7 pg-h/mL, 17.8 pg-h/mL, 17.9 pg-h/mL, 18 pg-h/mL, 18.1 pg-h/mL, 18.2 pg-h/mL, 18.3 pg-h/mL, 18.4 pg-h/mL, 18.5 pg-h/mL, 18.6 pg-h/mL, 18.7 pg-h/mL, 18.8 pg-h/mL, 18.9 pg-h/mL, 19 pg-h/mL, 19.1 pg-h/mL, 19.2 pg-h/mL, 19.3 pg-h/mL, 19.4 pg-h/mL, 19.5 pg-h/mL, 19.6 pg-h/mL, 19.7 pg-h/mL, 19.8 pg-h/mL, 19.9 pg-h/mL, 20 pg-h/mL, or the AUC (such as an AUC₀₋₆) may be 20.1 pg-h/mL. In certain embodiments, for example, the Cₘₐₓ may be 6 pg/mL, 6.1 pg/mL, 6.2 pg/mL, 6.3 pg/mL, 6.4 pg/mL, 6.5 pg/mL, 6.6 pg/mL, 6.7 pg/mL, 6.8 pg/mL, 6.9 pg/mL, 7 pg/mL, 7.1 pg/mL, 7.2 pg/mL, 7.3 pg/mL, 7.4 pg/mL, 7.5 pg/mL, 7.6 pg/mL, 7.7 pg/mL, 7.8 pg/mL, 7.9 pg/mL, 8 pg/mL, 8.1 pg/mL, 8.2 pg/mL, 8.3 pg/mL, 8.4 pg/mL, 8.5 pg/mL, 8.6 pg/mL, 8.7 pg/mL, 8.8 pg/mL, 8.9 pg/mL, 9 pg/mL, 9.1 pg/mL, 9.2 pg/mL, 9.3 pg/mL, 9.4 pg/mL, 9.5 pg/mL, 9.6 pg/mL, 9.7 pg/mL, 9.8 pg/mL, or the Cₘₐₓ may be 9.8 pg/mL.

G. In certain embodiments, for example, the tiotropium may be present in the pharmaceutical composition at a concentration in the range of 2.5-7 mcg/mL (for example the concentration of the tiotropium present in the pharmaceutical composition may be 2.5 mcg/mL, 2.6 mcg/mL, 2.7 mcg/mL, 2.8 mcg/mL, 2.9 mcg/mL, 3 mcg/mL, 3.1 mcg/mL, 3.2 mcg/mL, 3.3 mcg/mL, 3.4 mcg/mL, 3.5 mcg/mL, 3.6 mcg/mL, 3.7 mcg/mL, 3.8 mcg/mL, 3.9 mcg/mL, 4 mcg/mL, 4.1 mcg/mL, 4.2 mcg/mL, 4.3 mcg/mL, 4.4 mcg/mL, 4.5 mcg/mL, 4.6 mcg/mL, 4.7 mcg/mL, 4.8 mcg/mL, 4.9 mcg/mL, 5 mcg/mL, 5.1 mcg/mL, 5.2 mcg/mL, 5.3 mcg/mL, 5.4 mcg/mL, 5.5 mcg/mL, 5.6 mcg/mL, 5.7 mcg/mL, 5.8 mcg/mL, 5.9 mcg/mL, 6 mcg/mL, 6.1 mcg/mL, 6.2 mcg/mL, 6.3 mcg/mL, 6.4 mcg/mL, 6.5 mcg/mL, 6.6 mcg/mL, 6.7 mcg/mL, 6.8 mcg/mL, 6.9 mcg/mL, or the concentration of the tiotropium present in the pharmaceutical composition may be 7 mcg/mL).

Certain embodiments may provide, for example, a method of treating a patient (for example for treating a human COPD patient), comprising administering to the patient, once daily, a unit dose of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient (for example for treating a human COPD patient), comprising administering to the patient, once daily, a unit dose of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating COPD in a mammalian patient (for example a human patient) comprising administering to the patient a therapeutically effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg/mL of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating COPD in a mammalian patient (for example a human patient) comprising administering to the patient a therapeutically effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating COPD comprising administering to a mammal (for example a human) in need of such treatment an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating COPD comprising administering to a mammal (for example a human) in need of such treatment an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method for maintenance treatment of bronchospasm associated with COPD, and/or for reducing COPD exacerbations in a human patient, comprising administering to the patient a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method for maintenance treatment of bronchospasm associated with COPD, and/or for reducing COPD exacerbations in a human patient, comprising administering to the patient a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating or lessening the severity of bronchospasm associated with COPD and/or COPD exacerbations in a patient (for example a human patient), comprising administering to the patient a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating or lessening the severity of bronchospasm associated with COPD and/or COPD exacerbations in a patient (for example a human patient), comprising administering to the patient a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.1 wt. % citric acid 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient (for example a human patient) diagnosed as having COPD, the method comprising administering to the patient a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient (for example a human patient) diagnosed as having COPD, the method comprising administering to the patient a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with COPD, the method comprising administering to the patient an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with COPD, the method comprising administering to the patient an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) asthma, the method comprising administering to the patient an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) asthma, the method comprising administering to the patient an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) bronchial asthma, the method comprising administering to the patient an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) bronchial asthma, the method comprising administering to the patient an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) intrinsic asthma, the method comprising administering to the patient an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) intrinsic asthma, the method comprising administering to the patient an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) chronic bronchitis, the method comprising administering to the patient an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) chronic bronchitis, the method comprising administering to the patient an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) emphysema, the method comprising administering to the patient an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) emphysema, the method comprising administering to the patient an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) COPD, the method comprising administering to the patient an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) COPD, the method comprising administering to the patient an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) COPD exacerbations, the method comprising administering to the patient an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) COPD exacerbations, the method comprising administering to the patient an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) a combination of two or more of COPD, COPD exacerbations, asthma, pediatric asthma, bronchial asthma, allergic asthma, intrinsic asthma, chronic bronchitis, and emphysema, the method comprising administering to the patient an effective amount of a benzalkonium chloride-free, EDTA-free pharmaceutical composition comprising: i) 0.0007-0.001 wt. % (for example 0.0007-0.00088 wt. %) tiotropium (for example, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, or 0.001 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, wherein a 2 mL dose of the benzalkonium chloride-free, EDTA-free pharmaceutical composition containing 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium) dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0226 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a method of treating a patient with (or diagnosed with) a combination of two or more of COPD, COPD exacerbations, asthma, pediatric asthma, bronchial asthma, allergic asthma, intrinsic asthma, chronic bronchitis, and emphysema, the method comprising administering to the patient an effective amount of a benzalkonium chloride-free, EDTA-free pharmaceutical composition comprising: i) 0.00025-0.0007 wt. % tiotropium (for example, 0.00025 wt. %, 0.00026 wt. %, 0.00027 wt. %, 0.00028 wt. %, 0.00029 wt. %, 0.0003 wt. %, 0.00031 wt. %, 0.00032 wt. %, 0.00033 wt. %, 0.00034 wt. %, 0.00035 wt. %, 0.00036 wt. %, 0.00037 wt. %, 0.00038 wt. %, 0.00039 wt. %, 0.0004 wt. %, 0.00041 wt. %, 0.00042 wt. %, 0.00043 wt. %, 0.00044 wt. %, 0.00045 wt. %, 0.00046 wt. %, 0.00047 wt. %, 0.00048 wt. %, 0.00049 wt. %, 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, or 0.0007 wt. % tiotropium, or in the range of 0.00025-0.0005 wt. % tiotropium); ii) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); iii) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); iv) 0.6-0.9 wt. % sodium chloride (for example 0.6 wt. %, 0.7 wt. %, 0.8 wt. %, or 0.9 wt. % sodium chloride); and v) 97-99.2 wt. % water, a 2 mL dose of the benzalkonium chloride-free, EDTA-free pharmaceutical composition containing 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium) dispensed through a nebulizer is bioequivalent to a dose of a tiotropium solution containing 2.5 mcg of tiotropium dispensed through an inhaler, the tiotropium solution comprising water, tiotropium bromide monohydrate, edetate disodium, benzalkonium chloride and hydrochloric acid. In certain embodiments, for example, the inhaler-dispensed tiotropium solution may contain 0.0113 wt. % tiotropium, 0.01 wt. % benzalkonium chloride, 0.05 wt. % edetate disodium, water, and have a pH of 3.6. In certain embodiments, for example, the pharmaceutical composition may be complexing agent-free. In certain embodiments, for example, the pharmaceutical composition may be preservative-free. In certain embodiments, for example, the pharmaceutical composition may be complexing agent-free and preservative-free.

Certain embodiments may provide, for example, a pharmaceutical product comprising: i) a container having a volume in the range of 1-6 mL and; ii) a volume of a benzalkonium chloride-free, EDTA-free pharmaceutical composition (for example a sterile pharmaceutical composition) contained in the container, the pharmaceutical composition comprising: a) tiotropium; b) 0.08-0.6 wt. % sodium citrate (for example 0.08-0.2 wt. % sodium citrate, 0.2-0.6 wt. % sodium citrate, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % sodium citrate); c) 0.06-0.6 wt. % citric acid (for example in the range of 0.06 to 0.1 wt. % citric acid, in the range of 0.2-0.6 wt. % citric acid, 0.06 wt. %, 0.07 wt. %, 0.08 wt. %, 0.09 wt. %, 0.1 wt. %, 0.11 wt. %, 0.12 wt. %, 0.13 wt. %, 0.14 wt. %, 0.15 wt. %, 0.16 wt. %, 0.17 wt. %, 0.18 wt. %, 0.19 wt. %, 0.2 wt. %, 0.21 wt. %, 0.22 wt. %, 0.23 wt. %, 0.24 wt. %, 0.25 wt. %, 0.26 wt. %, 0.27 wt. %, 0.28 wt. %, 0.29 wt. %, 0.3 wt. %, 0.31 wt. %, 0.32 wt. %, 0.33 wt. %, 0.34 wt. %, 0.35 wt. %, 0.36 wt. %, 0.37 wt. %, 0.38 wt. %, 0.39 wt. %, 0.4 wt. %, 0.41 wt. %, 0.42 wt. %, 0.43 wt. %, 0.44 wt. %, 0.45 wt. %, 0.46 wt. %, 0.47 wt. %, 0.48 wt. %, 0.49 wt. %, 0.5 wt. %, 0.51 wt. %, 0.52 wt. %, 0.53 wt. %, 0.54 wt. %, 0.55 wt. %, 0.56 wt. %, 0.57 wt. %, 0.58 wt. %, 0.59 wt. %, or 0.6 wt. % citric acid); and d) at least 97 wt. % water, the pharmaceutical product administrable by a nebulizer to provide a nebulized dose of the pharmaceutical composition that is bioequivalent to a dose of a tiotropium solution administered through an inhaler. In certain embodiments, for example, the volume of the container may be greater than 2 mL and the volume of the pharmaceutical composition may be 2 mL. In certain embodiments, for example, the nebulized dose may contain 14-20 mcg (for example 14-17.6 mcg) of tiotropium (for example 14.0 mcg, 14.1 mcg, 14.2 mcg, 14.3 mcg, 14.4 mcg, 14.5 mcg, 14.6 mcg, 14.7 mcg, 14.8 mcg, 14.9 mcg, 15.0 mcg, 15.1 mcg, 15.2 mcg, 15.3 mcg, 15.4 mcg, 15.5 mcg, 15.6 mcg, 15.7 mcg, 15.8 mcg, 15.9 mcg, 16.0 mcg, 16.1 mcg, 16.2 mcg, 16.3 mcg, 16.4 mcg, 16.5 mcg, 16.6 mcg, 16.7 mcg, 16.8 mcg, 16.9 mcg, 17.0 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium). In certain embodiments, for example, the nebulized dose may contain 5-14 mcg of tiotropium (for example 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, or 14 mcg of tiotropium). In certain embodiments, for example, the tiotropium may be present in the pharmaceutical composition at a concentration in the range of 2.5-7 mcg/mL (for example the concentration of the tiotropium present in the pharmaceutical composition may be 2.5 mcg/mL, 2.6 mcg/mL, 2.7 mcg/mL, 2.8 mcg/mL, 2.9 mcg/mL, 3 mcg/mL, 3.1 mcg/mL, 3.2 mcg/mL, 3.3 mcg/mL, 3.4 mcg/mL, 3.5 mcg/mL, 3.6 mcg/mL, 3.7 mcg/mL, 3.8 mcg/mL, 3.9 mcg/mL, 4 mcg/mL, 4.1 mcg/mL, 4.2 mcg/mL, 4.3 mcg/mL, 4.4 mcg/mL, 4.5 mcg/mL, 4.6 mcg/mL, 4.7 mcg/mL, 4.8 mcg/mL, 4.9 mcg/mL, 5 mcg/mL, 5.1 mcg/mL, 5.2 mcg/mL, 5.3 mcg/mL, 5.4 mcg/mL, 5.5 mcg/mL, 5.6 mcg/mL, 5.7 mcg/mL, 5.8 mcg/mL, 5.9 mcg/mL, 6 mcg/mL, 6.1 mcg/mL, 6.2 mcg/mL, 6.3 mcg/mL, 6.4 mcg/mL, 6.5 mcg/mL, 6.6 mcg/mL, 6.7 mcg/mL, 6.8 mcg/mL, 6.9 mcg/mL, or the concentration of the tiotropium present in the pharmaceutical composition may be 7 mcg/mL). In certain embodiments, for example, the tiotropium may be present in the pharmaceutical composition at a concentration in the range of 7-8.8 mcg/mL or 7.8-10 mcg/mL (for example the concentration of the tiotropium present in the pharmaceutical composition may be 7 mcg/mL, 7.1 mcg/mL, 7.2 mcg/mL, 7.3 mcg/mL, 7.4 mcg/mL, 7.5 mcg/mL, 7.6 mcg/mL, 7.7 mcg/mL, 7.8 mcg/mL, 7.9 mcg/mL, 8 mcg/mL, 8.1 mcg/mL, 8.2 mcg/mL, 8.3 mcg/mL, 8.4 mcg/mL, 8.5 mcg/mL, 8.6 mcg/mL, 8.7 mcg/mL, 8.8 mcg/mL, 8.9 mcg/mL, 9 mcg/mL, 9.1 mcg/mL, 9.2 mcg/mL, 9.3 mcg/mL, 9.4 mcg/mL, 9.5 mcg/mL, 9.6 mcg/mL, 9.7 mcg/mL, 9.8 mcg/mL, 9.9 mcg/mL, or the concentration of the tiotropium present in the pharmaceutical composition may be 10 mcg/mL). In certain embodiments, for example, the pharmaceutical composition may be sterile. In certain embodiments, for example, the pharmaceutical composition may be preservative-free. In certain embodiments, for example, the pharmaceutical composition may be complexing agent-free. In certain embodiments, for example, the pharmaceutical composition may be complexing agent-free and preservative-free.

Each of the foregoing compositions, methods, and products may be inclusive of one or more (for example all) of the following embodiments. In certain embodiments, for example, the nebulized dose of the pharmaceutical composition may provide a blood plasma mean (for example a geometric or an arithmetic mean) steady-state area under the concentration-time curve (AUC) of tiotropium in the range of 80-125% (for example in the range of 85-120%, in the range of 90-110%, in the range of 95-105%, or in the range of 98-102% based on a 90% confidence interval or a 95% confidence interval) of an AUC of tiotropium of the inhaler-dispensed dose of the tiotropium solution. In certain embodiment, for example, the AUC may be an AUC to infinity. In certain embodiments, for example, the nebulized dose of the pharmaceutical composition may provide a Cₘₐₓ of tiotropium in the range of 80-125% (for example in the range of 85-120%, in the range of 90-110%, in the range of 95-105%, or in the range of 98-102% based on a 90% confidence interval or a 95% confidence interval) of a Cₘₐₓ of tiotropium of the inhaler-dispensed dose of the tiotropium solution. In certain embodiments, for example, the nebulized dose of the pharmaceutical composition may provide: (a) an AUC of tiotropium in the range of 80-125% (for example in the range of 85-120%, in the range of 90-110%, in the range of 95-105%, or in the range of 98-102% based on a 90% confidence interval or a 95% confidence interval) of an AUC of tiotropium of the inhaler-dispensed dose of the tiotropium solution; and (b) a Cₘₐₓ of tiotropium in the range of 80-125% (for example in the range of 85-120%, in the range of 90-110%, in the range of 95-105%, or in the range of 98-102% based on a 90% confidence interval or a 95% confidence interval) of a Cₘₐₓ of tiotropium of the inhaler-dispensed dose of the tiotropium solution. In certain embodiments, for example, the nebulized dose of the pharmaceutical composition may provide: (a) an AUC of tiotropium in the range of 80-125% (for example in the range of 85-120%, in the range of 90-110%, in the range of 95-105%, or in the range of 98-102% based on a 90% confidence interval or a 95% confidence interval) of an AUC of tiotropium of the inhaler-dispensed dose of the tiotropium solution; (b) a Cₘₐₓ of tiotropium in the range of 80-125% (for example in the range of 85-120%, in the range of 90-110%, in the range of 95-105%, or in the range of 98-102% based on a 90% confidence interval or a 95% confidence interval) of a Cₘₐₓ of tiotropium of the inhaler-dispensed dose of the tiotropium solution; and (c) a mean (for example a geometric or an arithmetic mean) steady-state time of maximum blood plasma concentration (tₘₐₓ) of tiotropium in the range of 80-125% (for example in the range of 85-120%, in the range of 90-110%, in the range of 95-105%, or in the range of 98-102% based on a 90% confidence interval or a 95% confidence interval) of a tₘₐₓ of tiotropium of the inhaler-dispensed dose of the tiotropium solution. In certain embodiments, for example, the nebulized dose of the pharmaceutical composition may provide: (a) an AUC of tiotropium in the range of 80-125% (for example in the range of 85-120%, in the range of 90-110%, in the range of 95-105%, or in the range of 98-102% based on a 90% confidence interval or a 95% confidence interval) of an AUC of tiotropium of the inhaler-dispensed dose of the tiotropium solution; (b) a Cₘₐₓ of tiotropium in the range of 80-125% (for example in the range of 85-120%, in the range of 90-110%, in the range of 95-105%, or in the range of 98-102% based on a 90% confidence interval or a 95% confidence interval) of a Cₘₐₓ of tiotropium of the inhaler-dispensed dose of the tiotropium solution; and (c) a mean (for example a geometric or an arithmetic mean) steady-state half-life (t_{1/2}) of tiotropium in the range of 80-125% (for example in the range of 85-120%, in the range of 90-110%, in the range of 95-105%, or in the range of 98-102% based on a 90% confidence interval or a 95% confidence interval) of a t_{1/2} of the inhaler-dispensed dose of the tiotropium solution. In certain embodiments, for example, the nebulized dose of the pharmaceutical composition may provide: (a) an AUC of tiotropium in the range of 80-125% (for example in the range of 85-120%, in the range of 90-110%, in the range of 95-105%, or in the range of 98-102% based on a 90% confidence interval or a 95% confidence interval) of an AUC of tiotropium of the inhaler-dispensed dose of the tiotropium solution; (b) a Cₘₐₓ of tiotropium in the range of 80-125% (for example in the range of 85-120%, in the range of 90-110%, in the range of 95-105%, or in the range of 98-102% based on a 90% confidence interval or a 95% confidence interval) of a Cₘₐₓ of tiotropium of the inhaler-dispensed dose of the tiotropium solution; and (c) a mean (for example a geometric or an arithmetic mean) steady-state elimination rate constant (λ_{Z}) of tiotropium in the range of 80-125% (for example in the range of 85-120%, in the range of 90-110%, in the range of 95-105%, or in the range of 98-102% based on a 90% confidence interval or a 95% confidence interval) of a λ_{Z} of tiotropium of the inhaler-dispensed dose of the tiotropium solution. In certain embodiments, for example, the nebulized dose of the pharmaceutical composition may provide: (a) an AUC of tiotropium in the range of 80-125% (for example in the range of 85-120%, in the range of 90-110%, in the range of 95-105%, or in the range of 98-102% based on a 90% confidence interval or a 95% confidence interval) of an AUC of tiotropium of the inhaler-dispensed dose of the tiotropium solution; (b) a Cₘₐₓ of tiotropium in the range of 80-125% (for example in the range of 85-120%, in the range of 90-110%, in the range of 95-105%, or in the range of 98-102% based on a 90% confidence interval or a 95% confidence interval) of a Cₘₐₓ of tiotropium of the inhaler-dispensed dose of the tiotropium solution; (c) a tₘₐₓ of tiotropium in the range of 80-125% (for example in the range of 85-120%, in the range of 90-110%, in the range of 95-105%, or in the range of 98-102% based on a 90% confidence interval or a 95% confidence interval) of a tₘₐₓ of tiotropium of the inhaler-dispensed dose of the tiotropium solution; (d) a t_{1/2} of tiotropium in the range of 80-125% (for example in the range of 85-120%, in the range of 90-110%, in the range of 95-105%, or in the range of 98-102% based on a 90% confidence interval or a 95% confidence interval) of a t_{1/2} of the inhaler-dispensed dose of the tiotropium solution; and (e) a λ_{Z} of tiotropium in the range of 80-125% (for example in the range of 85-120%, in the range of 90-110%, in the range of 95-105%, or in the range of 98-102% based on a 90% confidence interval or a 95% confidence interval) of a λ_{Z} of tiotropium of the inhaler-dispensed dose of the tiotropium solution.

Each of the foregoing compositions, methods, and products may be inclusive of one or more (for example all) of the following embodiments. In certain embodiments, for example, the nebulized dose of the pharmaceutical composition may be bioequivalent (for example therapeutically bioequivalent) to the inhaler-dispensed dose of the tiotropium solution. In certain embodiments, for example, the nebulized dose of the pharmaceutical composition may be bioequivalent (for example therapeutically bioequivalent) when compared to an AUC of tiotropium of the inhaler-dispensed dose of the tiotropium solution. In certain embodiments, for example, the AUC may be an AUC to infinity. In certain embodiments, for example, the nebulized dose of the pharmaceutical composition may be bioequivalent (for example therapeutically bioequivalent) when compared to a Cₘₐₓ of tiotropium of the inhaler-dispensed dose of the tiotropium solution. In certain embodiments, for example, the nebulized dose of the pharmaceutical composition may be bioequivalent (for example therapeutically bioequivalent) when compared to: (a) an AUC of tiotropium of the inhaler-dispensed dose of the tiotropium solution; and (b) a Cₘₐₓ of tiotropium of the inhaler-dispensed dose of the tiotropium solution. In certain embodiments, for example, the nebulized dose of the pharmaceutical composition may be bioequivalent (for example therapeutically bioequivalent) when compared to: (a) an AUC of tiotropium of the inhaler-dispensed dose of the tiotropium solution; (b) a Cₘₐₓ of tiotropium of the inhaler-dispensed dose of the tiotropium solution; and (c) a tₘₐₓ of tiotropium of the inhaler-dispensed dose of the tiotropium solution. In certain embodiments, for example, the nebulized dose of the pharmaceutical composition may be bioequivalent (for example therapeutically bioequivalent) when compared to: (a) an AUC of tiotropium of the inhaler-dispensed dose of the tiotropium solution; (b) a Cₘₐₓ of tiotropium of the inhaler-dispensed dose of the tiotropium solution; and (c) a t_{1/2} of tiotropium of the inhaler-dispensed dose of the tiotropium solution. In certain embodiments, for example, the nebulized dose of the pharmaceutical composition may be bioequivalent (for example therapeutically bioequivalent) when compared to: (a) an AUC of tiotropium of the inhaler-dispensed dose of the tiotropium solution; (b) a Cₘₐₓ of tiotropium of the inhaler-dispensed dose of the tiotropium solution; and (c) a λ_{Z} of tiotropium of the inhaler-dispensed dose of the tiotropium solution. In certain embodiments, for example, the nebulized dose of the pharmaceutical composition may be bioequivalent (for example therapeutically bioequivalent) when compared to: (a) an AUC of tiotropium of the inhaler-dispensed dose of the tiotropium solution; (b) a Cₘₐₓ of tiotropium of the inhaler-dispensed dose of the tiotropium solution; (c) a tₘₐₓ of tiotropium of the inhaler-dispensed dose of the tiotropium solution; (d) a t_{1/2} of tiotropium of the inhaler-dispensed dose of the tiotropium solution; and (e) a λ_{Z} of tiotropium of the inhaler-dispensed dose of the tiotropium solution.

Each of the foregoing compositions, methods, and products may be inclusive of one or more (for example all) of the following embodiments. In certain embodiments, for example, the nebulized dose of the pharmaceutical composition may be bioequivalent (for example bioequivalent (for example therapeutically bioequivalent)) when compared to: an improvement (for example an arithmetic or geometric mean improvement in one or more subjects) in trough forced expiratory volume in one second (trough FEV₁) compared to placebo of the inhaler-dispensed dose of the tiotropium solution. In certain embodiments, for example, the improvement may be determined based on once-daily use of the nebulized dose of the pharmaceutical composition for 48 weeks. In certain embodiments, for example, the nebulized dose of the pharmaceutical composition may be bioequivalent (for example therapeutically bioequivalent) one of the aqueous inhalation solutions disclosed by New Drug Application No. 021936 when administered according to the package insert for SPIRIVA RESPIMAT dated September, 2014. In certain embodiments, for example, the inhaler-dispensed dose of the tiotropium solution may comprise 5 mcg of tiotropium delivered in the form of liquid droplets (for example an aerosol). In certain embodiments, for example, the inhaler-dispensed dose of the tiotropium solution may comprise 2.5 mcg of tiotropium delivered in the form of liquid droplets (for example an aerosol). In certain embodiments, for example, the nebulized dose of the pharmaceutical composition may provide an improvement in trough FEV₁ in a patient that is in the range of 80-125% (for example in the range of 85-120%, in the range of 90-110%, in the range of 95-105%, or in the range of 98-102% based on a 90% confidence interval or a 95% confidence interval) of an improvement in trough FEV₁ of the inhaler-dispensed dose of the tiotropium solution. In certain embodiments, for example, the trough FEV₁ of the nebulized dose of the pharmaceutical composition and the trough FEV1 of the inhaler-dispensed dose of the tiotropium solution may be averages determined from a series of experiments (for example clinical trials on a number of test subjects).

Certain embodiments may provide, for example, a method of treating a patient, comprising administering a plurality of any one of the foregoing pharmaceutical products according to a prescribed treatment schedule to provide an improvement in trough FEV₁ of at least 8% (for example at least 10%, at least 12%, or at least 15%) or at least 80 mL (for example at least 100 mL) above placebo. In certain embodiments, for example, the improvement may be realized after 1 week of the prescribed treatment. In certain embodiments, for example, the prescribed treatment schedule may extend for at least 48 weeks. In certain embodiments, for example, the prescribed treatment may comprise once-daily administration by a nebulizer. In certain embodiments, for example, the improvement may be maintained during the at least 48 weeks of the prescribed treatment schedule.

In any of the foregoing embodiments, for example, the pharmaceutical composition may be nebulized by a nebulizer, wherein the nebulizer may be an air-driven jet nebulizer (for example a nebulizer connected to an air compressor) or a vibrating mesh nebulizer. In any of the foregoing embodiments, for example, the inhaler-dispensed dose may inhaled by an actuated droplet inhaler (for example a Respimat inhaler). In any of the foregoing embodiments, for example, the inhaler may comprise a 4.5 mL plastic container crimped into an aluminum cylinder.

Certain embodiments may provide, for example, a therapeutically effective unit dose of sterile nebulization solution, comprising a total tiotropium content of no more than 1 mcg of tiotropium (for example the total tiotropium content of 0.625 mcg or a total tiotropium content of 1 mcg), and a total water content of no more than 2 mL water. In certain embodiments, one or more than one (including for instance all) of the following embodiments may comprise each of the embodiments or parts thereof. In certain embodiments, for example, the unit dose may be therapeutically effective for the treatment of chronic obstructive pulmonary disease. In certain embodiments, for example, the unit dose may have a volume of 0.5 mL. In certain embodiments, for example, the unit dose may have a volume of 1 mL. In certain embodiments, for example, the unit dose may have a volume of 2 mL. In certain embodiments, for example, the nebulization solution may be aqueous (for example the nebulization solution may comprise at least 97 wt. % water). In certain embodiments, for example, the nebulization solution may be complexing agent-free and/or stabilization agent-free (for example the nebulization solution may be ethylenediaminetetraacetic acid-free and disodium edetate-free). In certain embodiments, for example, the nebulization solution may be preservative-free (for example the nebulization solution may be benzalkonium chloride-free). In certain embodiments, for example, the nebulization solution may comprise 0.00005-0.0001 wt. % tiotropium. In certain embodiments, for example, the nebulization solution may comprise citric acid (for example the nebulization solution may comprise 0.025-0.075 wt. %, 0.06-0.1 wt. %, or 0.2-0.6 wt. % citric acid). In certain embodiments, for example, the nebulization solution may comprise sodium citrate (for example the nebulization solution may comprise in the range of 0.08-0.2 wt. % sodium citrate or in the range of 0.2-0.6 wt. % sodium citrate). In certain embodiments, for example, the nebulization solution may comprise sodium chloride (for example the nebulization solution may comprise 0.5-1 wt. % sodium chloride). In certain embodiments, for example, the nebulization solution may have a pH in the range of 2.7-3.2.

Certain embodiments may provide, for example, a therapeutically effective unit dose of sterile nebulization solution, comprising a total tiotropium content in the range of 1-10 mcg of tiotropium (for example the total tiotropium content of 1.25 mcg, of 2.5 mcg, or a total tiotropium content of 5 mcg), and a total water content of no more than 1 mL water. In certain embodiments, one or more than one (including for instance all) of the following embodiments may comprise each of the embodiments or parts thereof. In certain embodiments, for example, the unit dose may be therapeutically effective for the treatment of chronic obstructive pulmonary disease. In certain embodiments, for example, the unit dose may have a volume of 0.25 mL. In certain embodiments, for example, the unit dose may have a volume of 0.5 mL. In certain embodiments, for example, the unit dose may have a volume of 0.75 mL. In certain embodiments, for example, the unit dose may have a volume in the range of 0.25-0.75 mL. In certain embodiments, for example, the nebulization solution may be aqueous (for example the nebulization solution may comprise at least 95 wt. % water or at least 97 wt. % water). In certain embodiments, for example, the nebulization solution may be complexing agent-free and/or stabilization agent-free (for example the nebulization solution may be ethylenediaminetetraacetic acid-free and disodium edetate-free). In certain embodiments, for example, the nebulization solution may be preservative-free (for example the nebulization solution may be benzalkonium chloride-free). In certain embodiments, for example, the nebulization solution may comprise in the range of 0.00005-0.002 wt. % tiotropium (for example in the range of 0.000125-0.001 wt. %). In certain embodiments, for example, the nebulization solution may comprise citric acid (for example the nebulization solution may comprise in the range of 0.025-0.075 wt. %, 0.06-0.1 wt. %, 0.2-0.6 wt. % citric acid or in the range of 0.075-0.3 wt. % citric acid). In certain embodiments, for example, the nebulization solution may comprise sodium citrate (for example the nebulization solution may comprise in the range of 0.08-0.2 wt. % sodium citrate, in the range of 0.2-0.6 wt. % sodium citrate, or in the range of 0.6-2.4 wt. % sodium citrate). In certain embodiments, for example, the nebulization solution may comprise sodium chloride (for example the nebulization solution may comprise 0.5-1 wt. % sodium chloride). In certain embodiments, for example, the nebulization solution may have a pH in the range of 2.7-3.2.

Certain embodiments may provide, for example, a therapeutically effective unit dose of sterile nebulization solution, comprising 0.0008-0.001 wt. % tiotropium, and at least 95 wt. % water. In certain embodiments, one or more than one (including for instance all) of the following embodiments may comprise each of the embodiments or parts thereof. In certain embodiments, for example, the unit dose may be therapeutically effective for the treatment of chronic obstructive pulmonary disease. In certain embodiments, for example, the unit dose may have a volume of 0.25 mL. In certain embodiments, for example, the unit dose may have a volume of 0.5 mL. In certain embodiments, for example, the unit dose may have a volume of 0.75 mL. In certain embodiments, for example, the unit dose may have a volume in the range of 0.25-0.75 mL. In certain embodiments, for example, the nebulization solution may be aqueous. In certain embodiments, for example, the nebulization solution may be complexing agent-free and/or stabilization agent-free (for example the nebulization solution may be ethylenediaminetetraacetic acid-free and disodium edetate-free). In certain embodiments, for example, the nebulization solution may be preservative-free (for example the nebulization solution may be benzalkonium chloride-free). In certain embodiments, for example, the nebulization solution may comprise in the range of 1.25-5 mcg of tiotropium. In certain embodiments, for example, the nebulization solution may comprise citric acid (for example the nebulization solution may comprise in the range of 0.025-0.075 wt. %, 0.06-0.1 wt. %, 0.2-0.6 wt. % citric acid or in the range of 0.075-0.3 wt. % citric acid). In certain embodiments, for example, the nebulization solution may comprise sodium citrate (for example the nebulization solution may comprise in the range of 0.08-0.2 wt. % sodium citrate, in the range of 0.2-0.6 wt. % sodium citrate, or in the range of 0.6-2.4 wt. % sodium citrate). In certain embodiments, for example, the nebulization solution may comprise sodium chloride (for example the nebulization solution may comprise 0.5-1 wt. % sodium chloride). In certain embodiments, for example, the nebulization solution may have a pH in the range of 2.7-3.2.

Certain embodiments may provide, for example, a therapeutically effective unit dose (for example a once-daily unit dose) of sterile nebulization solution comprising tiotropium. In certain embodiments, for example, the unit dose may comprise 1.25 mcg of tiotropium in 0.25 mL, 0.5 mL, 0.75 mL, 1 mL, or 2 mL of solution. In certain embodiments, for example, the unit dose may comprise 2.5 mcg of tiotropium in 0.25 mL, 0.5 mL, 0.75 mL, 1 mL, or 2 mL of solution. In certain embodiments, for example, the unit dose may comprise 5 mcg of tiotropium in 0.25 mL, 0.5 mL, 0.75 mL, 1 mL, or 2 mL of solution. In certain embodiments, for example, the tiotropium-comprising solution may have a concentration of 0.625 mcg/mL tiotropium, 1.25 mcg/mL tiotropium, 1.67 mcg/mL tiotropium, 2.5 mcg/mL tiotropium, 3.3 mcg/mL tiotropium, 5 mcg/mL tiotropium, 6.7 mcg/mL tiotropium, 10 mcg/mL tiotropium, or 20 mcg/mL tiotropium.

In any of the foregoing embodiments, for example, the unit dose may be aged for at least 18 months at a temperature of 25° C. and 40-75% relative humidity (for example 40% relative humidity or 60% relative humidity) under no-light conditions in a low-density polyethylene blow-fill-seal container, wherein the aged unit dose may pass one or more laboratory tests for sterility conducted in accordance with U.S. Pharmacopeia <71>. In certain embodiments, for example, the blow-fill-seal container may be aged while in a foil wrap. In certain embodiments, for example, the tiotropium nebulization solution may be iso-osmolal with respect to fluids in the lungs. In certain embodiments, for example, the tiotropium may be amorphous. In certain embodiments, for example, the tiotropium may be anhydrous. In certain embodiments, for example, the tiotropium may be amorphous and anhydrous.

Certain embodiments may provide, for example, a therapeutically effective unit dose of sterile nebulization solution, the sterile nebulization solution comprising: 0.002-0.006 wt. % sodium citrate, 0.0006-0.0001 wt. % citric acid, and at least 97 wt. % water. In certain further embodiments, for example, the sterile nebulization solution may be preservative-free, complexing agent-free, and have a pH in the range of 2.8-3.0. In certain further embodiments, for example, the unit dose may have a total volume of less than 2 mL and may comprise a total tiotropium content of 0.5 mcg to no more than 1 mcg of tiotropium. In certain embodiments, for example, the unit dose may have a total volume of less than 1 mL and may comprise a total tiotropium content of 1.25 mcg to no more than 10 mcg of tiotropium. In certain embodiments, for example, the unit dose may have a total volume of 0.25-0.75 mL and may comprise a total tiotropium content of 1.25 mcg to no more than 10 mcg of tiotropium. In certain embodiments, for example, the unit dose may have a total volume of 0.25-0.75 mL and may have tiotropium concentration in the range of 0.0005-0.001 wt. % (for example 0.0008-0.001 wt. %).

Certain embodiments may provide, for example, a method of treating, preventing, or ameliorating one or more symptoms of a bronchoconstriction-related disease or disorder (for example chronic obstructive pulmonary disease), comprising: nebulizing, by a nebulizer, one of the therapeutically effective unit doses one or more times per day (for example twice per day or three times per day). In certain embodiments, for example, the nebulizer may be a vibrating mesh nebulizer. In certain embodiments, for example, the nebulizer may be a hand-held, battery powered nebulizer. In certain embodiments, for example, the method may further comprise: providing the unit dose in a single use, blow-fill-seal container for use in the nebulizer. In certain embodiments, for example, the nebulized one of the therapeutically effective unit doses may form droplets having an average size in the range of 0.5-10 microns (for example 1-5 microns, 2-5 microns, 3-5 microns, or 1-6 microns when passed through a Pari LC Jet Plus Nebulizer connected to a Pari Master or a Pari VIOS compressor).

Certain embodiments may provide, for example, a method of increasing patient compliance with a therapeutically effective dosage regimen (for example a once daily dosage regimen). In certain embodiments, for example, patient compliance may be increased by reducing the amount of time required to deliver the dosage regimen (for example, the amount of time required to deliver the dosage regimen by nebulization). In certain embodiments, for example, the method may comprise nebulizing, by a vibrating mesh nebulizer, a therapeutically effective unit dose of tiotropium present in 1 mL or less sterile nebulization solution (for example a 1 mL solution containing tiotropium at a concentration of 5 mcg/mL or an 0.5 mL solution containing tiotropium at a concentration of 10 mcg/mL). In certain embodiments, for example, the vibrating mesh nebulizer may be handheld. In certain embodiments, for example, the vibrating mesh nebulizer may comprise a removable and/or disposable medicine cup. In certain embodiments, for example, the vibrating mesh nebulizer may nebulize all but no more than 0.05 mL of the sterile nebulization solution (for example, the vibrating mesh nebulizer may retain less than 0.05 mL residual sterile nebulization solution following administration of the therapeutically effective dosage regimen), for example all but no more than 0.02 mL of the sterile nebulization solution. In certain embodiments, for example, the 1 mL or less sterile nebulization solution may be nebulized in less than 10 minutes, for example less than 9 minutes, less than 8 minutes, less than 7 minutes, less than 6 minutes, less than 5 minutes, less than 4 minutes, less than 3 minutes, less than 2 minutes, or the 1 mL or less sterile nebulization solution may be nebulized in less than 1 minute. In certain embodiments, for example, the 1 mL or less sterile nebulization solution may be an 0.5 mL solution that may be nebulized in less than 10 minutes, for example less than 9 minutes, less than 8 minutes, less than 7 minutes, less than 6 minutes, less than 5 minutes, less than 4 minutes, less than 3 minutes, less than 2 minutes, or the 1 mL or less sterile nebulization solution may be nebulized in less than 1 minute. In certain embodiments, for example, the 1 mL or less sterile nebulization solution may be an 0.75 mL solution that may be nebulized in less than 10 minutes, for example less than 9 minutes, less than 8 minutes, less than 7 minutes, less than 6 minutes, less than 5 minutes, less than 4 minutes, less than 3 minutes, less than 2 minutes, or the 1 mL or less sterile nebulization solution may be nebulized in less than 1 minute. In certain embodiments, for example, the 1 mL or less sterile nebulization solution may be a 1 mL solution that may be nebulized in less than 10 minutes, for example less than 9 minutes, less than 8 minutes, less than 7 minutes, less than 6 minutes, less than 5 minutes, less than 4 minutes, less than 3 minutes, less than 2 minutes, or the 1 mL or less sterile nebulization solution may be nebulized in less than 1 minute.

Certain embodiments may provide, for example, a blow-fill-seal plastic ampoule containing a sterile, preservative-free, complexing agent-free pharmaceutical composition, the pharmaceutical composition comprising: 0.00005-0.0001 wt. % tiotropium, at least 0.4 wt. % sodium citrate (for example 0.4 wt. % sodium citrate), citric acid, and at least 97 wt. % water. In certain embodiments, for example, the sterile, preservative-free, complexing agent-free pharmaceutical composition may comprise no more than 0.06 wt. % citric acid (for example 0.06 wt % citric acid or 0.4 wt. % citric acid). In certain embodiments, for example, the sterile, preservative-free, complexing agent-free pharmaceutical composition of claim may be a unit dose for nebulizer inhalation therapy.

Certain embodiments may provide, for example, a blow-fill-seal plastic ampoule containing a sterile, preservative-free, complexing agent-free pharmaceutical composition, the pharmaceutical composition comprising: 0.0005-0.003 wt. % tiotropium (for example 0.0005-0.0015 wt. % tiotropium or 0.0008-0.001 wt. % tiotropium), at least 0.4 wt. % sodium citrate (for example 0.4 wt. % sodium citrate), citric acid, and at least 97 wt. % water. In certain embodiments, for example, the sterile, preservative-free, complexing agent-free pharmaceutical composition may comprise no more than 0.06 wt. % citric acid (for example 0.06 wt % citric acid or 0.4 wt. % citric acid). In certain embodiments, for example, the sterile, preservative-free, complexing agent-free pharmaceutical composition of claim may be a unit dose for nebulizer inhalation therapy.

Certain embodiments may provide, for example, a method to prepare a tiotropium nebulization solution, the method comprising: dissolving a quantity of tiotropium in a quantity of water, followed by adjusting the pH and/or osmolality of the tiotropium nebulization solution. In certain embodiments, for example, the tiotropium may be anhydrous. In certain embodiments, for example, the tiotropium may be amorphous. In certain embodiments, for example, the tiotropium may be anhydrous and amorphous.

Certain embodiments may provide, for example, a process to make a sterile tiotropium nebulization product, comprising: sterilizing a quantity of one of the tiotropium nebulization solution disclosed herein, injecting a therapeutically effective unit dose of the sterilized tiotropium nebulization solution into a sterile blow-fill-seal container, and sealing the sterile blow-fill-seal container, wherein the process may be exclusive of sterilization following the sealing. In certain embodiments, one or more than one (including for instance all) of the following embodiments may comprise each of the embodiments or parts thereof. In certain embodiments, for example, the process may be aseptic. In certain embodiments, for example, the process may be exclusive of heat sterilization following the sealing. In certain embodiments, for example, the sterilizing may be exclusive of heat sterilization prior to the injecting. In certain embodiments, for example, the sterilizing may comprise passing the tiotropium nebulization solution through a filter (i.e., sterile filtration). In certain further embodiments, for example, the sterile filtration may be performed prior to the injecting. In certain embodiments, for example, the therapeutically effective unit dose of the sterilized tiotropium nebulization solution may be further (for example increasingly or redundantly) sterilized by heat transfer from the blow-fill-seal container.

In certain embodiments, one or more than one (including for instance all) of the following embodiments may comprise each of the embodiments or parts thereof. In certain embodiments, for example, any one of the foregoing nebulization solutions, sterile nebulization solutions, pharmaceutical compositions (sterile, preservative-free, complexing agent-free), or tiotropium nebulization solutions (together, the “foregoing solutions”) may be precipitate-free. In certain embodiments, for example, any one of the foregoing solutions may be solids-free. In certain embodiments, for example, any one of the foregoing solutions may be preservative-free. In certain embodiments, for example, any one of the foregoing solutions may be benzalkonium chloride-free. In certain embodiments, for example, any one of the foregoing solutions may have a pH in the range of 2.5-3.2. In certain embodiments, for example, any one of the foregoing solutions may form less than 0.008 wt. % precipitate after being stored under dark conditions for 18 months at a temperature of 25° C. In certain embodiments, for example, any one of the foregoing solutions may be clear. In certain embodiments, for example, any one of the foregoing solutions may be colorless. In certain embodiments, for example, any one of the foregoing solutions may be foam-free. In certain embodiments, for example, any one of the foregoing solutions may be sterile and/or pyrogen-free. In certain embodiments, for example, a tiotropium solution may pass one or more laboratory tests for sterility conducted in accordance with U.S. Pharmacopeia <71>.

In certain embodiments, one or more than one (including for instance all) of the following embodiments may comprise each of the embodiments or parts thereof. In certain embodiments, for example, any one of the foregoing solutions may comprise an aqueous complexing agent-free, preservative-free aqueous buffer solution. In certain embodiments, for example, the complexing agent-free, preservative-free aqueous buffer solution may comprise citric acid. In certain embodiments, for example, the complexing agent-free, preservative-free aqueous buffer solution may comprise sodium citrate. In certain embodiments, for example, the complexing agent-free, preservative-free aqueous buffer solution may comprise sodium chloride. In certain embodiments, for example, the complexing agent-free, preservative-free aqueous buffer solution may be formed by combining in the range of 0.025-0.075 wt. % citric acid anhydrous (for example 0.06 wt. % citric acid anhydrous), relative to the total weight of the buffer solution, in the range of 0.2-0.6 wt. % sodium citrate dihydrate (for example 0.4 wt. % sodium citrate dihydrate), relative to the total weight of the buffer solution, in the range of 0.25-1.25 wt. % sodium chloride (for example 0.75 wt. % sodium chloride), relative to the total weight of the buffer solution, and water. In certain embodiments, for example, the pH of the complexing agent-free, preservative-free aqueous buffer solution may be adjusted to a pH in the range of 2.8-3 by adding a quantity of 1 N hydrochloric acid or 1 N sulfuric acid.

In certain embodiments, for example, the tiotropium solution may be clear. In certain embodiments, for example, the tiotropium solution may be colorless. In certain embodiments, for example, the tiotropium solution may be solids-free. In certain embodiments, for example, the tiotropium solution may be foam-free. In certain embodiments, for example, the tiotropium solution may be stable (for example: less than 5% of the tiotropium present in the tiotropium solution may decompose after at least 3 months at 25° C., no observable precipitate may be present in the tiotropium solution for at least 18 months at 25° C., the tiotropium solution may remain pharmaceutically acceptable for least 18 months at 25° C., the tiotropium solution may remain homogeneous for least 18 months at 25° C., the tiotropium solution may remain sterile for least 18 months at 25° C., and/or the tiotropium solution may remain solids-free for at least 18 months at 25° C.). In certain embodiments, for example, the tiotropium solution may have a tiotropium concentration in the range of 1-10 mcg/mL (for example 3.57 mcg/mL). In certain embodiments, for example, the tiotropium solution may be sterile. In certain embodiments, for example, the tiotropium solution may remain sterile for at least 18 months in a blow-fill-seal container sealed in a foil wrapping.

In any of the foregoing methods, for example, preparation of the drug product solution may comprise adjusting the pH of the diluted volume of the precipitate-free tiotropium solution, for example by adding citric acid, sodium citrate, and/or hydrochloric acid (for example by mixing the diluted volume of the precipitate-free tiotropium solution with an aqueous solution containing citric acid, sodium citrate, and/or hydrochloric acid). In certain embodiments, for example, the adjusted pH may be in the range of 2-6, for example in the range of 2.8-3.

Certain embodiments may provide, for example, a drug product comprising a sterile volume of any one of the foregoing solutions in a container (for example a plastic container such as a low-density polyethylene container). In certain embodiments, for example, the sterile volume of the any one of the foregoing solutions may be a single dose. In certain embodiments, for example, the container may be a single-use container. In certain embodiments, for example, the container may be sized to contain a single dose of the any one of the foregoing solutions. In certain embodiments, for example, the container may be formed by a blow-fill-seal process (for example a single use, blow-fill-seal container with a twist-off top formed by a blow-fill-seal process).

Certain embodiments may provide, for example, an aseptic process to make a drug product, comprising injecting a volume of any one of the foregoing tiotropium solutions into a sterile blow-fill-seal container or partially formed container (for example a container in the process of being formed), and sealing the sterile blow-fill-seal container. In certain embodiments, for example, the blow-fill-seal container may be formed from molten plastic at a temperature above where microorganisms can survive in a sterile chamber, whereby the formed blow-fill-seal container may be sterile. In certain embodiments, for example, the molten plastic may be at a temperature in the range of 170-193° C. In certain embodiments, for example, the process may be exclusive of sterilization (for example exclusive of heat sterilization) following the sealing. In certain further embodiments, for example, the blow-fill-seal container and the tiotropium solution disposed therein may be sterile (for example due to being formed in a sterile chamber), and may remain sterile for an extended period (for example at least 24 months) even if the process may be exclusive of heat sterilization following the sealing. In certain embodiments, for example, the blow-fill-seal container may be impermeable to air. In certain further embodiments, for example, the blow-fill-seal container may be permeable to air or at least one component thereof. In certain embodiments, for example, the blow-fill-seal container may be impermeable to microorganisms, inclusive of bacteria and viruses. In certain embodiments, for example, the process may further comprise impressing an electronic batch data code (for example cipher text) onto the sterile blow-fill-seal container. In certain embodiments, for example, the electronic batch data referenced by the electronic batch data code may be configured for distributed ledger processing (for example the electronic batch data or ciphertext thereof may be included in a blockchain or other distributed ledger technology). In certain embodiments, for example, a blow-fill-seal method may comprise impressing an electronic batch data code (for example ciphertext) onto a blow-fill-seal container. In certain embodiments, for example, the electronic batch data referenced by the electronic batch data code may be configured for distributed ledger processing (for example the electronic batch data or a ciphertext thereof may be included in a blockchain or another distributed ledger).

In certain further embodiments, for example, the process may further comprise sterile filtration of the any one of the tiotropium solutions disclosed herein prior to introduction to the blow-fill-seal container. In certain embodiments, for example, the sterile filtration may comprise microfiltration using one or more filters (for example the one or more filters may comprise a membrane filter). In certain embodiments, for example, the one or more filters may have a pore size of less than 1 micron, for example less than 0.5 microns, less than 0.2 microns, less than 0.1 micron, or the one or more filters may have a pore size of less than 0.05 microns. In certain embodiment, for example, the one or more filters may have a pore size in the range of 0.05-1 microns, for example in the range of 0.1-0.5 microns, in the range of 0.15-0.3 microns, or the one or more filters may have a pore size in the range of 0.19-0.25 microns. In certain embodiments, for example, any of the foregoing one or more filters may be a pre-filter. In certain embodiments, for example, the one or more filters may have a pore size of less than 100 nm, for example less than 75 nm, less than 50 nm, less than 30 nm, or the one or more filters may have a pore size of less than 20 nm. In certain embodiment, for example, the one or more filters may have a pore size in the range of 1-100 nm, for example in the range of 10-75 nm, in the range of 20-50 nm, or the one or more filters may have a pore size in the range of 30-50 nm. In certain embodiments, for example, the one or more filters may comprise mixed cellulose ester. In certain embodiments, for example, the one or more filters may comprise polyethersulfone (PES). In certain embodiments, for example, the one or more filters may comprise a pre-sterilized disposable unit. In certain further embodiments, for example, the pre-sterilized disposable unit may be provided in a sealed package. In certain embodiments, for example, the one or more filters may be sterilized (for example by autoclaving at a temperature that does not damage the filter). In certain embodiments, for example, the one or more filters may be tested for integrity and/or sterility prior to use. In certain embodiments, for example, the one or more filters may be tested for integrity and/or sterility after use. In certain embodiments, for example, the sterile filtration may be performed in a clean room. In certain further embodiments, for example, the clean room may have no more than 1,000,000 particles, per cubic meter, having a size larger than 0.5 microns, for example no more than 100,000 particles per cubic meter, for example no more than 10,000 particles per cubic meter, for example no more than 5,000 particles per cubic meter, no more than 2,500 particles per cubic meter, no more than 2,000 particles per cubic meter, no more than 1,000 particles per cubic meter, no more than 100 particles per cubic meter, no more than 50 particles per cubic meter, no more than 12 particles per cubic meter, or the clean room may have fewer than 1000 particles, per cubic meter, having a size larger than 0.5 microns. In certain embodiments, for example, the clean room may be an ISO class 5 clean room. In certain embodiments, for example, the clean room may be an ISO class 4 clean room. In certain embodiments, for example, the clean room may be an ISO class 3 clean room. In certain embodiments, for example, the clean room may be an ISO class 2 clean room. In certain embodiments, for example, the clean room may be an ISO class 1 clean room. In certain embodiments, for example, air entering the clean room may be passed through a high-efficiency particulate air (HEPA) filter prior to entering the clean room. In certain embodiments, for example, air entering the clean room may be passed through a high-efficiency particulate air (HEPA) filter prior to entering the clean room. In certain embodiments, for example, air entering the clean room may be passed through an ultra-low particulate air (ULPA) filter.

In certain embodiments, for example, the sterile filtration of the tiotropium solution may remove at least 95 wt. % of microorganisms or other pathogens present in the tiotropium solution prior to the sterile filtration, for example at least 99 wt. %, 99.9 wt. %, 99.99 wt. %, 99.999 wt. %, 99.9999 wt. %, 99.99999 wt. %, 99.999999 wt. %, 99.9999999 wt. %, or the sterile filtration of the tiotropium solution may remove at least 99.99999999 wt. % of microorganisms or other pathogens present in the tiotropium solution prior to the sterile filtration.

Certain embodiments may provide, for example, a method of treating, preventing, or ameliorating one or more symptoms of a bronchoconstriction-related disease or disorder, comprising nebulizing, by a nebulizer, a unit dose of any one of the tiotropium solutions disclosed herein. In certain embodiments, for example, the unit dose may be nebulized by a vibrating mesh nebulizer. In certain embodiments, for example, the nebulizer may be a hand-held, battery-powered nebulizer (for example a hand-held, battery powered, vibrating mesh nebulizer). In certain embodiments, for example, the method may further comprise providing the unit dose to the nebulizer from a single use, blow-fill-seal container.

### DETAILED DESCRIPTION OF THE INVENTION

Certain embodiments may provide, for example, a tiotropium solution. In certain embodiments, for example, the tiotropium solution may be a drug product. In certain embodiments, for example, the drug product may be a sterile nebulizable pharmaceutical solution for inhalation via nebulization. In certain embodiments, for example, the drug product may be a sterile ophthalmic solution. In certain embodiments, for example, the drug product may be a sterile nasal spray. In certain embodiments, for example, the drug product may be a sterile topical solution. In certain embodiments, for example, the drug product may be a sterile solution suitable for intravenous injection or injection into tissue. In certain embodiments, for example, the tiotropium solution may be a solution that may be dried (for example spray dried or freeze-dried) to form a sterile powdered drug product (for example a powdered drug product suitable for delivery by nasal or pulmonary inhalation).

Certain embodiments may provide, for example, a tiotropium solution providing a therapeutically effective unit dose of tiotropium. In certain embodiments, for example, the therapeutically effective unit dose of tiotropium may be in the range of 0.25 mcg to no more than 30 mcg, for example in the range of 0.25 mcg to no more than 0.5 mcg, in the range of 0.5 mcg to no more than 0.75 mcg, in the range of 0.75 mcg to no more than 1 mcg, in the range of 1 mcg to no more than 1.25 mcg, in the range of 1.25 mcg to no more than 2 mcg, in the range of 2 mcg to no more than 2.25 mcg, in the range of 2.25 mcg to no more than 2.5 mcg, in the range of 2.5 mcg to no more than 2.625 mcg, in the range of 2.625 mcg to no more than 3 mcg, in the range of 3 mcg to no more than 5 mcg, in the range of 5 mcg to no more than 8 mcg, in the range of 8 mcg to no more than 10 mcg, in the range of 10 mcg to no more than 12 mcg, in the range of 12 mcg to no more than 15 mcg, in the range of 15 mcg to no more than 18 mcg, in the range of 18 mcg to no more than 21 mcg, in the range of 21 mcg to no more than 25 mcg, in the range of 25 mcg to no more than 28 mcg, or the therapeutically effective unit dose of tiotropium may be in the range of 28 mcg to no more than 30 mcg. In certain embodiments, for example, the therapeutically effective unit dose of tiotropium may be less than 30 mcg, less than 28 mcg, less than 25 mcg, less than 21 mcg, less than 18 mcg, less than 15 mcg, less than 12 mcg, less than 10 mcg, less than 8 mcg, less than 5 mcg, less than 2.5 mcg, less than 2.125 mcg, less than 1.25 mcg, or the therapeutically effective unit dose of tiotropium may be less than 0.625 mcg. In certain embodiments, for example, a unit dose of tiotropium (for example a unit dose of tiotropium in a sterile, preservative-free, complexing agent-free pharmaceutical composition, such as a sterile, preservative-free, complexing agent-free pharmaceutical composition contained in a container such as a blow-fill-seal container, and/or a therapeutically effective unit dose, and/or a unit dose bioequivalent to an inhaler-delivered (and/or inhaler-dispensed) dose of a tiotropium solution containing 2.5 or 5 mcg of tiotropium) may be 2.5 mcg, 2.6 mcg, 2.7 mcg, 2.8 mcg, 2.9 mcg, 3.0 mcg, 3.1 mcg, 3.2 mcg, 3.3 mcg, 3.4 mcg, 3.5 mcg, 3.6 mcg, 3.7 mcg, 3.8 mcg, 3.9 mcg, 4.0 mcg, 4.1 mcg, 4.2 mcg, 4.3 mcg, 4.4 mcg, 4.5 mcg, 4.6 mcg, 4.7 mcg, 4.8 mcg, 4.9 mcg, 5 mcg, 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, 14 mcg, 14.1 mcg, 14.2 mcg, 14.3 mcg, 14.4 mcg, 14.5 mcg, 14.6 mcg, 14.7 mcg, 14.8 mcg, 14.9 mcg, 15 mcg, 15.1 mcg, 15.2 mcg, 15.3 mcg, 15.4 mcg, 15.5 mcg, 15.6 mcg, 15.7 mcg, 15.8 mcg, 15.9 mcg, 16 mcg, 16.1 mcg, 16.2 mcg, 16.3 mcg, 16.4 mcg, 16.5 mcg, 16.6 mcg, 16.7 mcg, 16.8 mcg, 16.9 mcg, 17 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or the unit dose of tiotropium may be 20.0 mcg.

In certain embodiments, for example, the tiotropium solution may comprise tiotropium at a concentration in the range of 0.000025-0.012 wt. %, for example in the range of 0.000025-0.00005 wt. %, in the range of 0.00005-0.000075 wt. %, in the range of 0.000075-0.0001 wt. %, in the range of 0.0001-0.000125 wt. %, in the range of 0.000125-0.0002 wt. %, in the range of 0.0002-0.000225 wt. %, in the range of 0.000225-0.00025 wt. %, in the range of 0.00025-0.0002625 wt. %, in the range of 0.0002625-0.0003 wt. %, in the range of 0.0003-0.0005 wt. %, in the range of 0.0005-0.0008 wt. %, in the range of 0.0008-0.001 wt. %, in the range of 0.001-0.0015 wt. %, in the range of 0.0015-0.002 wt. %, in the range of 0.002-0.0025 wt. %, in the range of 0.0025-0.004 wt. %, in the range of 0.004-0.005 wt. %, in the range of 0.005-0.006 wt. %, in the range of 0.006-0.007 wt. %, in the range of 0.007-0.008 wt. %, in the range of 0.008-0.009 wt. %, in the range of 0.009-0.01 wt. %, in the range of 0.01-0.011 wt. %, or the tiotropium solution may comprise tiotropium at a concentration in the range of 0.011-0.012 wt. %. In certain embodiments, for example, the tiotropium solution may comprise tiotropium at a concentration of less than 0.012 wt. %, less than 0.011 wt. %, less than 0.01 wt. %, less than 0.009 wt. %, less than 0.008 wt. %, less than 0.007 wt. %, less than 0.006 wt. %, less than 0.005 wt. %, less than 0.004 wt. %, less than 0.0025 wt. %, less than 0.002 wt. %, less than 0.0015 wt. %, less than 0.001 wt. %, less than 0.0008 wt. %, less than 0.0005 wt. %, less than 0.00025 wt. %, less than 0.0002125 wt. %, less than 0.000126 wt. %, or the tiotropium solution may comprise tiotropium at a concentration of less than 0.0000625 wt. %. In certain embodiments, for example, the tiotropium solution (for example a sterile, preservative-free, complexing agent-free tiotropium solution, such as a sterile, preservative-free, complexing agent-free tiotropium solution contained in a container such as a blow-fill-seal container, and/or a therapeutically effective unit dose, and/or a unit dose bioequivalent to an inhaler-delivered (and/or inhaler-dispensed) dose of a tiotropium solution containing 2.5 or 5 mcg of tiotropium) may have a concentration of 0.001 wt. %, 0.00102 wt. %, 0.00104 wt. %, 0.00106 wt. %, 0.00108 wt. %, 0.0011 wt. %, 0.00112 wt. %, 0.00114 wt. %, 0.00116 wt. %, 0.00118 wt. %, 0.0012 wt. %, 0.00122 wt. %, 0.00124 wt. %, 0.00126 wt. %, 0.00128 wt. %, 0.0013 wt. %, 0.00132 wt. %, 0.00134 wt. %, 0.00136 wt. %, 0.00138 wt. %, 0.0014 wt. %, 0.00142 wt. %, 0.00144 wt. %, 0.00146 wt. %, 0.00148 wt. %, 0.0015 wt. %, 0.00152 wt. %, 0.00154 wt. %, 0.00156 wt. %, 0.00158 wt. %, 0.0016 wt. %, 0.00162 wt. %, 0.00164 wt. %, 0.00166 wt. %, 0.00168 wt. %, 0.0017 wt. %, 0.00172 wt. %, 0.00174 wt. %, 0.00176 wt. %, 0.00178 wt. %, 0.0018 wt. %, 0.00182 wt. %, 0.00184 wt. %, 0.00186 wt. %, 0.00188 wt. %, 0.0019 wt. %, 0.00192 wt. %, 0.00194 wt. %, 0.00196 wt. %, 0.00198 wt. %, 0.002 wt. %, 0.00202 wt. %, 0.00204 wt. %, 0.00206 wt. %, 0.00208 wt. %, 0.0021 wt. %, 0.00212 wt. %, 0.00214 wt. %, 0.00216 wt. %, 0.00218 wt. %, 0.0022 wt. %, 0.00222 wt. %, 0.00224 wt. %, 0.00226 wt. %, 0.00228 wt. %, 0.0023 wt. %, 0.00232 wt. %, 0.00234 wt. %, 0.00236 wt. %, 0.00238 wt. %, 0.0024 wt. %, 0.00242 wt. %, 0.00244 wt. %, 0.00246 wt. %, 0.00248 wt. %, 0.0025 wt. %. 0.00252 wt. %, 0.00254 wt. %. 0.00256 wt. %, 0.00258 wt. %. 0.0026 wt. %, 0.00262 wt. %. 0.00264 wt. %, 0.00266 wt. %. 0.00268 wt. %, 0.0027 wt. %. 0.00272 wt. %, 0.00274 wt. %. 0.00276 wt. %, 0.00278 wt. %. 0.0028 wt. %, 0.00282 wt. %. 0.00284 wt. %, 0.00286 wt. %. 0.00288 wt. %, 0.0029 wt. %. 0.00292 wt. %, 0.00294 wt. %. 0.00296 wt. %, 0.00298 wt. %. 0.003 wt. %, 0.00302 wt. %. 0.00304 wt. %, 0.00306 wt. %. 0.00308 wt. %, 0.0031 wt. %. 0.00312 wt. %, 0.00314 wt. %. 0.00316 wt. %, 0.00318 wt. %. 0.0032 wt. %, 0.00322 wt. %. 0.00324 wt. %, 0.00326 wt. %. 0.00328 wt. %, 0.0033 wt. %. 0.00332 wt. %, 0.00334 wt. %. 0.00336 wt. %, 0.00338 wt. %. 0.0034 wt. %, 0.00342 wt. %. 0.00344 wt. %, 0.00346 wt. %. 0.00348 wt. %, 0.0035 wt. %. 0.00352 wt. %, 0.00354 wt. %. 0.00356 wt. %, 0.00358 wt. %. 0.0036 wt. %, 0.00362 wt. %. 0.00364 wt. %, 0.00366 wt. %. 0.00368 wt. %, 0.0037 wt. %. 0.00372 wt. %, 0.00374 wt. %. 0.00376 wt. %, 0.00378 wt. %. 0.0038 wt. %, 0.00382 wt. %. 0.00384 wt. %, 0.00386 wt. %. 0.00388 wt. %, 0.0039 wt. %. 0.00392 wt. %, 0.00394 wt. %. 0.00396 wt. %, 0.00398 wt. %, or the tiotropium solution may have a concentration of 0.004 wt. %. In certain embodiments, for example, the tiotropium solution (for example a sterile, preservative-free, complexing agent-free tiotropium solution, such as a sterile, preservative-free, complexing agent-free tiotropium solution contained in a container such as a blow-fill-seal container, and/or a therapeutically effective unit dose, and/or a unit dose bioequivalent to an inhaler-delivered (and/or inhaler-dispensed) dose of a tiotropium solution containing 2.5 or 5 mcg of tiotropium) may have a concentration of 0.0005 wt. %, 0.00051 wt. %, 0.00052 wt. %, 0.00053 wt. %, 0.00054 wt. %, 0.00055 wt. %, 0.00056 wt. %, 0.00057 wt. %, 0.00058 wt. %, 0.00059 wt. %, 0.0006 wt. %, 0.00061 wt. %, 0.00062 wt. %, 0.00063 wt. %, 0.00064 wt. %, 0.00065 wt. %, 0.00066 wt. %, 0.00067 wt. %, 0.00068 wt. %, 0.00069 wt. %, 0.0007 wt. %, 0.00071 wt. %, 0.00072 wt. %, 0.00073 wt. %, 0.00074 wt. %, 0.00075 wt. %, 0.00076 wt. %, 0.00077 wt. %, 0.00078 wt. %, 0.00079 wt. %, 0.0008 wt. %, 0.00081 wt. %, 0.00082 wt. %, 0.00083 wt. %, 0.00084 wt. %, 0.00085 wt. %, 0.00086 wt. %, 0.00087 wt. %, 0.00088 wt. %, 0.00089 wt. %, 0.0009 wt. %, 0.00091 wt. %, 0.00092 wt. %, 0.00093 wt. %, 0.00094 wt. %, 0.00095 wt. %, 0.00096 wt. %, 0.00097 wt. %, 0.00098 wt. %, 0.00099 wt. %, 0.001 wt. %, 0.00101 wt. %, 0.00102 wt. %, 0.00103 wt. %, 0.00104 wt. %, 0.00105 wt. %, 0.00106 wt. %, 0.00107 wt. %, 0.00108 wt. %, 0.00109 wt. %, 0.0011 wt. %, 0.00111 wt. %, 0.00112 wt. %, 0.00113 wt. %, 0.00114 wt. %, 0.00115 wt. %, 0.00116 wt. %, 0.00117 wt. %, 0.00118 wt. %, 0.00119 wt. %, 0.0012 wt. %, 0.00121 wt. %, 0.00122 wt. %, 0.00123 wt. %, 0.00124 wt. %, 0.00125 wt. %, 0.00126 wt. %, 0.00127 wt. %, 0.00128 wt. %, 0.00129 wt. %, 0.0013 wt. %, 0.00131 wt. %, 0.00132 wt. %, 0.00133 wt. %, 0.00134 wt. %, 0.00135 wt. %, 0.00136 wt. %, 0.00137 wt. %, 0.00138 wt. %, 0.00139 wt. %, 0.0014 wt. %, 0.00141 wt. %, 0.00142 wt. %, 0.00143 wt. %, 0.00144 wt. %, 0.00145 wt. %, 0.00146 wt. %, 0.00147 wt. %, 0.00148 wt. %, 0.00149 wt. %, 0.0015 wt. %, 0.00151 wt. %, 0.00152 wt. %, 0.00153 wt. %, 0.00154 wt. %, 0.00155 wt. %, 0.00156 wt. %, 0.00157 wt. %, 0.00158 wt. %, 0.00159 wt. %, 0.0016 wt. %, 0.00161 wt. %, 0.00162 wt. %, 0.00163 wt. %, 0.00164 wt. %, 0.00165 wt. %, 0.00166 wt. %, 0.00167 wt. %, 0.00168 wt. %, 0.00169 wt. %, 0.0017 wt. %, 0.00171 wt. %, 0.00172 wt. %, 0.00173 wt. %, 0.00174 wt. %, 0.00175 wt. %, 0.00176 wt. %, 0.00177 wt. %, 0.00178 wt. %, 0.00179 wt. %, 0.0018 wt. %, 0.00181 wt. %, 0.00182 wt. %, 0.00183 wt. %, 0.00184 wt. %, 0.00185 wt. %, 0.00186 wt. %, 0.00187 wt. %, 0.00188 wt. %, 0.00189 wt. %, 0.0019 wt. %, 0.00191 wt. %, 0.00192 wt. %, 0.00193 wt. %, 0.00194 wt. %, 0.00195 wt. %, 0.00196 wt. %, 0.00197 wt. %, 0.00198 wt. %, 0.00199 wt. %, or the tiotropium solution may have a concentration of 0.002 wt. %. In certain embodiments, for example, the tiotropium solution (for example a sterile, preservative-free, complexing agent-free tiotropium solution, such as a sterile, preservative-free, complexing agent-free tiotropium solution contained in a container such as a blow-fill-seal container, and/or a therapeutically effective unit dose, and/or a unit dose bioequivalent to an inhaler-delivered (and/or inhaler-dispensed) dose of a tiotropium solution containing 2.5 or 5 mcg of tiotropium) may have a concentration of 0.00025 wt. %, 0.000255 wt. %, 0.00026 wt. %, 0.000265 wt. %, 0.00027 wt. %, 0.000275 wt. %, 0.00028 wt. %, 0.000285 wt. %, 0.00029 wt. %, 0.000295 wt. %, 0.0003 wt. %, 0.000305 wt. %, 0.00031 wt. %, 0.000315 wt. %, 0.00032 wt. %, 0.000325 wt. %, 0.00033 wt. %, 0.000335 wt. %, 0.00034 wt. %, 0.000345 wt. %, 0.00035 wt. %, 0.000355 wt. %, 0.00036 wt. %, 0.000365 wt. %, 0.00037 wt. %, 0.000375 wt. %, 0.00038 wt. %, 0.000385 wt. %, 0.00039 wt. %, 0.000395 wt. %, 0.0004 wt. %, 0.000405 wt. %, 0.00041 wt. %, 0.000415 wt. %, 0.00042 wt. %, 0.000425 wt. %, 0.00043 wt. %, 0.000435 wt. %, 0.00044 wt. %, 0.000445 wt. %, 0.00045 wt. %, 0.000455 wt. %, 0.00046 wt. %, 0.000465 wt. %, 0.00047 wt. %, 0.000475 wt. %, 0.00048 wt. %, 0.000485 wt. %, 0.00049 wt. %, 0.000495 wt. %, 0.0005 wt. %, 0.000505 wt. %, 0.00051 wt. %, 0.000515 wt. %, 0.00052 wt. %, 0.000525 wt. %, 0.00053 wt. %, 0.000535 wt. %, 0.00054 wt. %, 0.000545 wt. %, 0.00055 wt. %, 0.000555 wt. %, 0.00056 wt. %, 0.000565 wt. %, 0.00057 wt. %, 0.000575 wt. %, 0.00058 wt. %, 0.000585 wt. %, 0.00059 wt. %, 0.000595 wt. %, 0.0006 wt. %, 0.000605 wt. %, 0.00061 wt. %, 0.000615 wt. %, 0.00062 wt. %, 0.000625 wt. %, 0.00063 wt. %, 0.000635 wt. %, 0.00064 wt. %, 0.000645 wt. %, 0.00065 wt. %, 0.000655 wt. %, 0.00066 wt. %, 0.000665 wt. %, 0.00067 wt. %, 0.000675 wt. %, 0.00068 wt. %, 0.000685 wt. %, 0.00069 wt. %, 0.000695 wt. %, 0.0007 wt. %, 0.000705 wt. %, 0.00071 wt. %, 0.000715 wt. %, 0.00072 wt. %, 0.000725 wt. %, 0.00073 wt. %, 0.000735 wt. %, 0.00074 wt. %, 0.000745 wt. %, 0.00075 wt. %, 0.000755 wt. %, 0.00076 wt. %, 0.000765 wt. %, 0.00077 wt. %, 0.000775 wt. %, 0.00078 wt. %, 0.000785 wt. %, 0.00079 wt. %, 0.000795 wt. %, 0.0008 wt. %, 0.000805 wt. %, 0.00081 wt. %, 0.000815 wt. %, 0.00082 wt. %, 0.000825 wt. %, 0.00083 wt. %, 0.000835 wt. %, 0.00084 wt. %, 0.000845 wt. %, 0.00085 wt. %, 0.000855 wt. %, 0.00086 wt. %, 0.000865 wt. %, 0.00087 wt. %, 0.000875 wt. %, 0.00088 wt. %, 0.000885 wt. %, 0.00089 wt. %, 0.000895 wt. %, 0.0009 wt. %, 0.000905 wt. %, 0.00091 wt. %, 0.000915 wt. %, 0.00092 wt. %, 0.000925 wt. %, 0.00093 wt. %, 0.000935 wt. %, 0.00094 wt. %, 0.000945 wt. %, 0.00095 wt. %, 0.000955 wt. %, 0.00096 wt. %, 0.000965 wt. %, 0.00097 wt. %, 0.000975 wt. %, 0.00098 wt. %, 0.000985 wt. %, 0.00099 wt. %, 0.000995 wt. %, or the tiotropium solution may have a concentration of 0.001 wt. %.

In certain embodiments, for example, the tiotropium solution may provide tiotropium at a concentration in the range of 0.25-10 mcg/mL, for example in the range of 0.25-0.5 mcg/mL, in the range of 0.5-0.75 mcg/mL, in the range of 0.75-1 mcg/mL, in the range of 1-1.25 mcg/mL, in the range of 1.25-2 mcg/mL, in the range of 2-2.25 mcg/mL, in the range of 2.25-2.5 mcg/mL, in the range of 2.5-2.625 mcg/mL, in the range of 2.625-3 mcg/mL, in the range of 3-5 mcg/mL, in the range of 5-8 mcg/mL, in the range of 8-10 mcg/mL, in the range of 10-12 mcg/mL, in the range of 12-15 mcg/mL, in the range of 15-18 mcg/mL, in the range of 18-21 mcg/mL, in the range of 21-25 mcg/mL, in the range of 25-28 mcg/mL, the tiotropium solution may provide tiotropium at a concentration in the range of 28-30 mcg/mL. In certain embodiments, for example, the tiotropium solution may comprise tiotropium at a concentration of less than 30 mcg/mL, less than 28 mcg/mL, less than 25 mcg/mL, less than 21 mcg/mL, less than 18 mcg/mL, less than 15 mcg/mL, less than 12 mcg/mL, less than 10 mcg/mL, less than 8 mcg/mL, less than 5 mcg/mL, less than 2.5 mcg/mL, less than 2.125 mcg/mL, less than 1.25 mcg/mL, or the tiotropium solution may comprise tiotropium at a concentration of less than 0.625 mcg/mL.

In certain embodiments, for example, the tiotropium solution may comprise tiotropium at a concentration of 5 mcg/mL, 5.1 mcg/mL, 5.2 mcg/mL, 5.3 mcg/mL, 5.4 mcg/mL, 5.5 mcg/mL, 5.6 mcg/mL, 5.7 mcg/mL, 5.8 mcg/mL, 5.9 mcg/mL, 6 mcg/mL, 6.1 mcg/mL, 6.2 mcg/mL, 6.3 mcg/mL, 6.4 mcg/mL, 6.5 mcg/mL, 6.6 mcg/mL, 6.7 mcg/mL, 6.8 mcg/mL, 6.9 mcg/mL, 7 mcg/mL, 7.1 mcg/mL, 7.2 mcg/mL, 7.3 mcg/mL, 7.4 mcg/mL, 7.5 mcg/mL, 7.6 mcg/mL, 7.7 mcg/mL, 7.8 mcg/mL, 7.9 mcg/mL, 8 mcg/mL, 8.1 mcg/mL, 8.2 mcg/mL, 8.3 mcg/mL, 8.4 mcg/mL, 8.5 mcg/mL, 8.6 mcg/mL, 8.7 mcg/mL, 8.8 mcg/mL, 8.9 mcg/mL, 9 mcg/mL, 9.1 mcg/mL, 9.2 mcg/mL, 9.3 mcg/mL, 9.4 mcg/mL, 9.5 mcg/mL, 9.6 mcg/mL, 9.7 mcg/mL, 9.8 mcg/mL, 9.9 mcg/mL, 10 mcg/mL, 10.1 mcg/mL, 10.2 mcg/mL, 10.3 mcg/mL, 10.4 mcg/mL, 10.5 mcg/mL, 10.6 mcg/mL, 10.7 mcg/mL, 10.8 mcg/mL, 10.9 mcg/mL, 11 mcg/mL, 11.1 mcg/mL, 11.2 mcg/mL, 11.3 mcg/mL, 11.4 mcg/mL, 11.5 mcg/mL, 11.6 mcg/mL, 11.7 mcg/mL, 11.8 mcg/mL, 11.9 mcg/mL, 12 mcg/mL, 12.1 mcg/mL, 12.2 mcg/mL, 12.3 mcg/mL, 12.4 mcg/mL, 12.5 mcg/mL, 12.6 mcg/mL, 12.7 mcg/mL, 12.8 mcg/mL, 12.9 mcg/mL, 13 mcg/mL, 13.1 mcg/mL, 13.2 mcg/mL, 13.3 mcg/mL, 13.4 mcg/mL, 13.5 mcg/mL, 13.6 mcg/mL, 13.7 mcg/mL, 13.8 mcg/mL, 13.9 mcg/mL, 14 mcg/mL, 14.1 mcg/mL, 14.2 mcg/mL, 14.3 mcg/mL, 14.4 mcg/mL, 14.5 mcg/mL, 14.6 mcg/mL, 14.7 mcg/mL, 14.8 mcg/mL, 14.9 mcg/mL, 15 mcg/mL, 15.1 mcg/mL, 15.2 mcg/mL, 15.3 mcg/mL, 15.4 mcg/mL, 15.5 mcg/mL, 15.6 mcg/mL, 15.7 mcg/mL, 15.8 mcg/mL, 15.9 mcg/mL, 16 mcg/mL, 16.1 mcg/mL, 16.2 mcg/mL, 16.3 mcg/mL, 16.4 mcg/mL, 16.5 mcg/mL, 16.6 mcg/mL, 16.7 mcg/mL, 16.8 mcg/mL, 16.9 mcg/mL, 17 mcg/mL, 17.1 mcg/mL, 17.2 mcg/mL, 17.3 mcg/mL, 17.4 mcg/mL, 17.5 mcg/mL, 17.6 mcg/mL, 17.7 mcg/mL, 17.8 mcg/mL, 17.9 mcg/mL, 18 mcg/mL, 18.1 mcg/mL, 18.2 mcg/mL, 18.3 mcg/mL, 18.4 mcg/mL, 18.5 mcg/mL, 18.6 mcg/mL, 18.7 mcg/mL, 18.8 mcg/mL, 18.9 mcg/mL, 19 mcg/mL, 19.1 mcg/mL, 19.2 mcg/mL, 19.3 mcg/mL, 19.4 mcg/mL, 19.5 mcg/mL, 19.6 mcg/mL, 19.7 mcg/mL, 19.8 mcg/mL, 19.9 mcg/mL, or the tiotropium solution may comprise tiotropium at a concentration of 20 mcg/mL.

In certain embodiments, for example, the tiotropium solution may be provided in a therapeutic unit dose volume of 0.25 mL, the therapeutic unit dose volume comprising tiotropium at a concentration in the range of 0.25-10 mcg/0.25 mL, for example in the range of 0.25-0.5 mcg/0.25 mL, in the range of 0.5-0.75 mcg/0.25 mL, in the range of 0.75-1 mcg/0.25 mL, in the range of 1-1.25 mcg/0.25 mL, in the range of 1.25-2 mcg/0.25 mL, in the range of 2-2.25 mcg/0.25 mL, in the range of 2.25-2.5 mcg/0.25 mL, in the range of 2.5-2.625 mcg/0.25 mL, in the range of 2.625-3 mcg/0.25 mL, in the range of 3-5 mcg/0.25 mL, in the range of 5-8 mcg/0.25 mL, or the therapeutic (for example therapeutically effective or minimal therapeutically effective) unit dose volume may comprise tiotropium at a concentration in the range of 8-10 mcg/0.25 mL. In certain embodiments, for example, the tiotropium solution may be provided in a therapeutic unit dose volume of 0.25 mL, the therapeutic unit dose volume comprising tiotropium at a concentration of less than 10 mcg/0.25 mL, less than 8 mcg/0.25 mL, less than 5 mcg/0.25 mL, less than 2.5 mcg/0.25 mL, less than 2.125 mcg/0.25 mL, less than 1.25 mcg/0.25 mL, or the therapeutic unit dose volume may comprise tiotropium at a concentration of less than 0.625 mcg/0.25 mL. In certain embodiments, for example, the tiotropium solution may be provided in a therapeutic unit dose volume of 0.25 mL, the therapeutic unit dose volume comprising tiotropium at a concentration of 5 mcg/0.25 mL, 5.1 mcg/0.25 mL, 5.2 mcg/0.25 mL, 5.3 mcg/0.25 mL, 5.4 mcg/0.25 mL, 5.5 mcg/0.25 mL, 5.6 mcg/0.25 mL, 5.7 mcg/0.25 mL, 5.8 mcg/0.25 mL, 5.9 mcg/0.25 mL, 6 mcg/0.25 mL, 6.1 mcg/0.25 mL, 6.2 mcg/0.25 mL, 6.3 mcg/0.25 mL, 6.4 mcg/0.25 mL, 6.5 mcg/0.25 mL, 6.6 mcg/0.25 mL, 6.7 mcg/0.25 mL, 6.8 mcg/0.25 mL, 6.9 mcg/0.25 mL, 7 mcg/0.25 mL, 7.1 mcg/0.25 mL, 7.2 mcg/0.25 mL, 7.3 mcg/0.25 mL, 7.4 mcg/0.25 mL, 7.5 mcg/0.25 mL, 7.6 mcg/0.25 mL, 7.7 mcg/0.25 mL, 7.8 mcg/0.25 mL, 7.9 mcg/0.25 mL, 8 mcg/0.25 mL, 8.1 mcg/0.25 mL, 8.2 mcg/0.25 mL, 8.3 mcg/0.25 mL, 8.4 mcg/0.25 mL, 8.5 mcg/0.25 mL, 8.6 mcg/0.25 mL, 8.7 mcg/0.25 mL, 8.8 mcg/0.25 mL, 8.9 mcg/0.25 mL, 9 mcg/0.25 mL, 9.1 mcg/0.25 mL, 9.2 mcg/0.25 mL, 9.3 mcg/0.25 mL, 9.4 mcg/0.25 mL, 9.5 mcg/0.25 mL, 9.6 mcg/0.25 mL, 9.7 mcg/0.25 mL, 9.8 mcg/0.25 mL, 9.9 mcg/0.25 mL, 10 mcg/0.25 mL, 10.1 mcg/0.25 mL, 10.2 mcg/0.25 mL, 10.3 mcg/0.25 mL, 10.4 mcg/0.25 mL, 10.5 mcg/0.25 mL, 10.6 mcg/0.25 mL, 10.7 mcg/0.25 mL, 10.8 mcg/0.25 mL, 10.9 mcg/0.25 mL, 11 mcg/0.25 mL, 11.1 mcg/0.25 mL, 11.2 mcg/0.25 mL, 11.3 mcg/0.25 mL, 11.4 mcg/0.25 mL, 11.5 mcg/0.25 mL, 11.6 mcg/0.25 mL, 11.7 mcg/0.25 mL, 11.8 mcg/0.25 mL, 11.9 mcg/0.25 mL, 12 mcg/0.25 mL, 12.1 mcg/0.25 mL, 12.2 mcg/0.25 mL, 12.3 mcg/0.25 mL, 12.4 mcg/0.25 mL, 12.5 mcg/0.25 mL, 12.6 mcg/0.25 mL, 12.7 mcg/0.25 mL, 12.8 mcg/0.25 mL, 12.9 mcg/0.25 mL, 13 mcg/0.25 mL, 13.1 mcg/0.25 mL, 13.2 mcg/0.25 mL, 13.3 mcg/0.25 mL, 13.4 mcg/0.25 mL, 13.5 mcg/0.25 mL, 13.6 mcg/0.25 mL, 13.7 mcg/0.25 mL, 13.8 mcg/0.25 mL, 13.9 mcg/0.25 mL, 14 mcg/0.25 mL, 14.1 mcg/0.25 mL, 14.2 mcg/0.25 mL, 14.3 mcg/0.25 mL, 14.4 mcg/0.25 mL, 14.5 mcg/0.25 mL, 14.6 mcg/0.25 mL, 14.7 mcg/0.25 mL, 14.8 mcg/0.25 mL, 14.9 mcg/0.25 mL, 15 mcg/0.25 mL, 15.1 mcg/0.25 mL, 15.2 mcg/0.25 mL, 15.3 mcg/0.25 mL, 15.4 mcg/0.25 mL, 15.5 mcg/0.25 mL, 15.6 mcg/0.25 mL, 15.7 mcg/0.25 mL, 15.8 mcg/0.25 mL, 15.9 mcg/0.25 mL, 16 mcg/0.25 mL, 16.1 mcg/0.25 mL, 16.2 mcg/0.25 mL, 16.3 mcg/0.25 mL, 16.4 mcg/0.25 mL, 16.5 mcg/0.25 mL, 16.6 mcg/0.25 mL, 16.7 mcg/0.25 mL, 16.8 mcg/0.25 mL, 16.9 mcg/0.25 mL, 17 mcg/0.25 mL, 17.1 mcg/0.25 mL, 17.2 mcg/0.25 mL, 17.3 mcg/0.25 mL, 17.4 mcg/0.25 mL, 17.5 mcg/0.25 mL, 17.6 mcg/0.25 mL, 17.7 mcg/0.25 mL, 17.8 mcg/0.25 mL, 17.9 mcg/0.25 mL, 18 mcg/0.25 mL, 18.1 mcg/0.25 mL, 18.2 mcg/0.25 mL, 18.3 mcg/0.25 mL, 18.4 mcg/0.25 mL, 18.5 mcg/0.25 mL, 18.6 mcg/0.25 mL, 18.7 mcg/0.25 mL, 18.8 mcg/0.25 mL, 18.9 mcg/0.25 mL, 19 mcg/0.25 mL, 19.1 mcg/0.25 mL, 19.2 mcg/0.25 mL, 19.3 mcg/0.25 mL, 19.4 mcg/0.25 mL, 19.5 mcg/0.25 mL, 19.6 mcg/0.25 mL, 19.7 mcg/0.25 mL, 19.8 mcg/0.25 mL, 19.9 mcg/0.25 mL, or tiotropium at a concentration of 20 mcg/0.25 mL.

In certain embodiments, for example, the tiotropium solution may be provided in a therapeutic unit dose volume of 0.5 mL, the therapeutic unit dose volume comprising tiotropium at a concentration in the range of 0.25-10 mcg/0.5 mL, for example in the range of 0.25-0.5 mcg/0.5 mL, in the range of 0.5-0.75 mcg/0.5 mL, in the range of 0.75-1 mcg/0.5 mL, in the range of 1-1.25 mcg/0.5 mL, in the range of 1.25-2 mcg/0.5 mL, in the range of 2-2.25 mcg/0.5 mL, in the range of 2.25-2.5 mcg/0.5 mL, in the range of 2.5-2.625 mcg/0.5 mL, in the range of 2.625-3 mcg/0.5 mL, in the range of 3-5 mcg/0.5 mL, in the range of 5-8 mcg/0.5 mL, or the therapeutic unit dose volume may comprise tiotropium at a concentration in the range of 8-10 mcg/0.5 mL. In certain embodiments, for example, the tiotropium solution may be provided in a therapeutic unit dose volume of 0.5 mL, the therapeutic unit dose volume comprising tiotropium at a concentration of less than 10 mcg/0.5 mL, less than 8 mcg/0.5 mL, less than 5 mcg/0.5 mL, less than 2.5 mcg/0.5 mL, less than 2.125 mcg/0.5 mL, less than 1.25 mcg/0.5 mL, or the therapeutic unit dose volume may comprise tiotropium at a concentration of less than 0.625 mcg/0.5 mL. In certain embodiments, for example, the tiotropium solution may be provided in a therapeutic unit dose volume of 0.5 mL, the therapeutic unit dose volume comprising tiotropium at a concentration of 5 mcg/0.5 mL, 5.1 mcg/0.5 mL, 5.2 mcg/0.5 mL, 5.3 mcg/0.5 mL, 5.4 mcg/0.5 mL, 5.5 mcg/0.5 mL, 5.6 mcg/0.5 mL, 5.7 mcg/0.5 mL, 5.8 mcg/0.5 mL, 5.9 mcg/0.5 mL, 6 mcg/0.5 mL, 6.1 mcg/0.5 mL, 6.2 mcg/0.5 mL, 6.3 mcg/0.5 mL, 6.4 mcg/0.5 mL, 6.5 mcg/0.5 mL, 6.6 mcg/0.5 mL, 6.7 mcg/0.5 mL, 6.8 mcg/0.5 mL, 6.9 mcg/0.5 mL, 7 mcg/0.5 mL, 7.1 mcg/0.5 mL, 7.2 mcg/0.5 mL, 7.3 mcg/0.5 mL, 7.4 mcg/0.5 mL, 7.5 mcg/0.5 mL, 7.6 mcg/0.5 mL, 7.7 mcg/0.5 mL, 7.8 mcg/0.5 mL, 7.9 mcg/0.5 mL, 8 mcg/0.5 mL, 8.1 mcg/0.5 mL, 8.2 mcg/0.5 mL, 8.3 mcg/0.5 mL, 8.4 mcg/0.5 mL, 8.5 mcg/0.5 mL, 8.6 mcg/0.5 mL, 8.7 mcg/0.5 mL, 8.8 mcg/0.5 mL, 8.9 mcg/0.5 mL, 9 mcg/0.5 mL, 9.1 mcg/0.5 mL, 9.2 mcg/0.5 mL, 9.3 mcg/0.5 mL, 9.4 mcg/0.5 mL, 9.5 mcg/0.5 mL, 9.6 mcg/0.5 mL, 9.7 mcg/0.5 mL, 9.8 mcg/0.5 mL, 9.9 mcg/0.5 mL, 10 mcg/0.5 mL, 10.1 mcg/0.5 mL, 10.2 mcg/0.5 mL, 10.3 mcg/0.5 mL, 10.4 mcg/0.5 mL, 10.5 mcg/0.5 mL, 10.6 mcg/0.5 mL, 10.7 mcg/0.5 mL, 10.8 mcg/0.5 mL, 10.9 mcg/0.5 mL, 11 mcg/0.5 mL, 11.1 mcg/0.5 mL, 11.2 mcg/0.5 mL, 11.3 mcg/0.5 mL, 11.4 mcg/0.5 mL, 11.5 mcg/0.5 mL, 11.6 mcg/0.5 mL, 11.7 mcg/0.5 mL, 11.8 mcg/0.5 mL, 11.9 mcg/0.5 mL, 12 mcg/0.5 mL, 12.1 mcg/0.5 mL, 12.2 mcg/0.5 mL, 12.3 mcg/0.5 mL, 12.4 mcg/0.5 mL, 12.5 mcg/0.5 mL, 12.6 mcg/0.5 mL, 12.7 mcg/0.5 mL, 12.8 mcg/0.5 mL, 12.9 mcg/0.5 mL, 13 mcg/0.5 mL, 13.1 mcg/0.5 mL, 13.2 mcg/0.5 mL, 13.3 mcg/0.5 mL, 13.4 mcg/0.5 mL, 13.5 mcg/0.5 mL, 13.6 mcg/0.5 mL, 13.7 mcg/0.5 mL, 13.8 mcg/0.5 mL, 13.9 mcg/0.5 mL, 14 mcg/0.5 mL, 14.1 mcg/0.5 mL, 14.2 mcg/0.5 mL, 14.3 mcg/0.5 mL, 14.4 mcg/0.5 mL, 14.5 mcg/0.5 mL, 14.6 mcg/0.5 mL, 14.7 mcg/0.5 mL, 14.8 mcg/0.5 mL, 14.9 mcg/0.5 mL, 15 mcg/0.5 mL, 15.1 mcg/0.5 mL, 15.2 mcg/0.5 mL, 15.3 mcg/0.5 mL, 15.4 mcg/0.5 mL, 15.5 mcg/0.5 mL, 15.6 mcg/0.5 mL, 15.7 mcg/0.5 mL, 15.8 mcg/0.5 mL, 15.9 mcg/0.5 mL, 16 mcg/0.5 mL, 16.1 mcg/0.5 mL, 16.2 mcg/0.5 mL, 16.3 mcg/0.5 mL, 16.4 mcg/0.5 mL, 16.5 mcg/0.5 mL, 16.6 mcg/0.5 mL, 16.7 mcg/0.5 mL, 16.8 mcg/0.5 mL, 16.9 mcg/0.5 mL, 17 mcg/0.5 mL, 17.1 mcg/0.5 mL, 17.2 mcg/0.5 mL, 17.3 mcg/0.5 mL, 17.4 mcg/0.5 mL, 17.5 mcg/0.5 mL, 17.6 mcg/0.5 mL, 17.7 mcg/0.5 mL, 17.8 mcg/0.5 mL, 17.9 mcg/0.5 mL, 18 mcg/0.5 mL, 18.1 mcg/0.5 mL, 18.2 mcg/0.5 mL, 18.3 mcg/0.5 mL, 18.4 mcg/0.5 mL, 18.5 mcg/0.5 mL, 18.6 mcg/0.5 mL, 18.7 mcg/0.5 mL, 18.8 mcg/0.5 mL, 18.9 mcg/0.5 mL, 19 mcg/0.5 mL, 19.1 mcg/0.5 mL, 19.2 mcg/0.5 mL, 19.3 mcg/0.5 mL, 19.4 mcg/0.5 mL, 19.5 mcg/0.5 mL, 19.6 mcg/0.5 mL, 19.7 mcg/0.5 mL, 19.8 mcg/0.5 mL, 19.9 mcg/0.5 mL, or tiotropium at a concentration of 20 mcg/0.5 mL.

In certain embodiments, for example, the tiotropium solution may be provided in a therapeutic unit dose volume of 0.75 mL, the therapeutic unit dose volume comprising tiotropium at a concentration in the range of 0.25-10 mcg/0.75 mL, for example in the range of 0.25-0.5 mcg/0.75 mL, in the range of 0.5-0.75 mcg/0.75 mL, in the range of 0.75-1 mcg/0.75 mL, in the range of 1-1.25 mcg/0.75 mL, in the range of 1.25-2 mcg/0.75 mL, in the range of 2-2.25 mcg/0.75 mL, in the range of 2.25-2.5 mcg/0.75 mL, in the range of 2.5-2.625 mcg/0.75 mL, in the range of 2.625-3 mcg/0.75 mL, in the range of 3-5 mcg/0.75 mL, in the range of 5-8 mcg/0.75 mL, or the therapeutic unit dose volume may comprise tiotropium at a concentration in the range of 8-10 mcg/0.75 mL. In certain embodiments, for example, the tiotropium solution may be provided in a therapeutic unit dose volume of 0.75 mL, the therapeutic unit dose volume comprising tiotropium at a concentration of less than 10 mcg/0.75 mL, less than 8 mcg/0.75 mL, less than 5 mcg/0.75 mL, less than 2.5 mcg/0.75 mL, less than 2.125 mcg/0.75 mL, less than 1.25 mcg/0.75 mL, or the therapeutic unit dose volume may comprise tiotropium at a concentration of less than 0.625 mcg/0.75 mL. In certain embodiments, for example, the tiotropium solution may be provided in a therapeutic unit dose volume of 0.75 mL, the therapeutic unit dose volume comprising tiotropium at a concentration of 5 mcg/0.75 mL, 5.1 mcg/0.75 mL, 5.2 mcg/0.75 mL, 5.3 mcg/0.75 mL, 5.4 mcg/0.75 mL, 5.5 mcg/0.75 mL, 5.6 mcg/0.75 mL, 5.7 mcg/0.75 mL, 5.8 mcg/0.75 mL, 5.9 mcg/0.75 mL, 6 mcg/0.75 mL, 6.1 mcg/0.75 mL, 6.2 mcg/0.75 mL, 6.3 mcg/0.75 mL, 6.4 mcg/0.75 mL, 6.5 mcg/0.75 mL, 6.6 mcg/0.75 mL, 6.7 mcg/0.75 mL, 6.8 mcg/0.75 mL, 6.9 mcg/0.75 mL, 7 mcg/0.75 mL, 7.1 mcg/0.75 mL, 7.2 mcg/0.75 mL, 7.3 mcg/0.75 mL, 7.4 mcg/0.75 mL, 7.5 mcg/0.75 mL, 7.6 mcg/0.75 mL, 7.7 mcg/0.75 mL, 7.8 mcg/0.75 mL, 7.9 mcg/0.75 mL, 8 mcg/0.75 mL, 8.1 mcg/0.75 mL, 8.2 mcg/0.75 mL, 8.3 mcg/0.75 mL, 8.4 mcg/0.75 mL, 8.5 mcg/0.75 mL, 8.6 mcg/0.75 mL, 8.7 mcg/0.75 mL, 8.8 mcg/0.75 mL, 8.9 mcg/0.75 mL, 9 mcg/0.75 mL, 9.1 mcg/0.75 mL, 9.2 mcg/0.75 mL, 9.3 mcg/0.75 mL, 9.4 mcg/0.75 mL, 9.5 mcg/0.75 mL, 9.6 mcg/0.75 mL, 9.7 mcg/0.75 mL, 9.8 mcg/0.75 mL, 9.9 mcg/0.75 mL, 10 mcg/0.75 mL, 10.1 mcg/0.75 mL, 10.2 mcg/0.75 mL, 10.3 mcg/0.75 mL, 10.4 mcg/0.75 mL, 10.5 mcg/0.75 mL, 10.6 mcg/0.75 mL, 10.7 mcg/0.75 mL, 10.8 mcg/0.75 mL, 10.9 mcg/0.75 mL, 11 mcg/0.75 mL, 11.1 mcg/0.75 mL, 11.2 mcg/0.75 mL, 11.3 mcg/0.75 mL, 11.4 mcg/0.75 mL, 11.5 mcg/0.75 mL, 11.6 mcg/0.75 mL, 11.7 mcg/0.75 mL, 11.8 mcg/0.75 mL, 11.9 mcg/0.75 mL, 12 mcg/0.75 mL, 12.1 mcg/0.75 mL, 12.2 mcg/0.75 mL, 12.3 mcg/0.75 mL, 12.4 mcg/0.75 mL, 12.5 mcg/0.75 mL, 12.6 mcg/0.75 mL, 12.7 mcg/0.75 mL, 12.8 mcg/0.75 mL, 12.9 mcg/0.75 mL, 13 mcg/0.75 mL, 13.1 mcg/0.75 mL, 13.2 mcg/0.75 mL, 13.3 mcg/0.75 mL, 13.4 mcg/0.75 mL, 13.5 mcg/0.75 mL, 13.6 mcg/0.75 mL, 13.7 mcg/0.75 mL, 13.8 mcg/0.75 mL, 13.9 mcg/0.75 mL, 14 mcg/0.75 mL, 14.1 mcg/0.75 mL, 14.2 mcg/0.75 mL, 14.3 mcg/0.75 mL, 14.4 mcg/0.75 mL, 14.5 mcg/0.75 mL, 14.6 mcg/0.75 mL, 14.7 mcg/0.75 mL, 14.8 mcg/0.75 mL, 14.9 mcg/0.75 mL, 15 mcg/0.75 mL, 15.1 mcg/0.75 mL, 15.2 mcg/0.75 mL, 15.3 mcg/0.75 mL, 15.4 mcg/0.75 mL, 15.5 mcg/0.75 mL, 15.6 mcg/0.75 mL, 15.7 mcg/0.75 mL, 15.8 mcg/0.75 mL, 15.9 mcg/0.75 mL, 16 mcg/0.75 mL, 16.1 mcg/0.75 mL, 16.2 mcg/0.75 mL, 16.3 mcg/0.75 mL, 16.4 mcg/0.75 mL, 16.5 mcg/0.75 mL, 16.6 mcg/0.75 mL, 16.7 mcg/0.75 mL, 16.8 mcg/0.75 mL, 16.9 mcg/0.75 mL, 17 mcg/0.75 mL, 17.1 mcg/0.75 mL, 17.2 mcg/0.75 mL, 17.3 mcg/0.75 mL, 17.4 mcg/0.75 mL, 17.5 mcg/0.75 mL, 17.6 mcg/0.75 mL, 17.7 mcg/0.75 mL, 17.8 mcg/0.75 mL, 17.9 mcg/0.75 mL, 18 mcg/0.75 mL, 18.1 mcg/0.75 mL, 18.2 mcg/0.75 mL, 18.3 mcg/0.75 mL, 18.4 mcg/0.75 mL, 18.5 mcg/0.75 mL, 18.6 mcg/0.75 mL, 18.7 mcg/0.75 mL, 18.8 mcg/0.75 mL, 18.9 mcg/0.75 mL, 19 mcg/0.75 mL, 19.1 mcg/0.75 mL, 19.2 mcg/0.75 mL, 19.3 mcg/0.75 mL, 19.4 mcg/0.75 mL, 19.5 mcg/0.75 mL, 19.6 mcg/0.75 mL, 19.7 mcg/0.75 mL, 19.8 mcg/0.75 mL, 19.9 mcg/0.75 mL, or tiotropium at a concentration of 20 mcg/0.75 mL.

In certain embodiments, for example, the tiotropium solution may be provided in a therapeutic unit dose volume of 1 mL, the therapeutic unit dose volume comprising tiotropium at a concentration in the range of 0.25-10 mcg/1 mL, for example in the range of 0.25-0.5 mcg/1 mL, in the range of 0.5-0.75 mcg/1 mL, in the range of 0.75-1 mcg/1 mL, in the range of 1-1.25 mcg/1 mL, in the range of 1.25-2 mcg/1 mL, in the range of 2-2.25 mcg/1 mL, in the range of 2.25-2.5 mcg/1 mL, in the range of 2.5-2.625 mcg/1 mL, in the range of 2.625-3 mcg/1 mL, in the range of 3-5 mcg/1 mL, in the range of 5-8 mcg/1 mL, or the therapeutic unit dose volume may comprise tiotropium at a concentration in the range of 8-10 mcg/1 mL. In certain embodiments, for example, the tiotropium solution may be provided in a therapeutic unit dose volume of 1 mL, the therapeutic unit dose volume comprising tiotropium at a concentration of less than 10 mcg/1 mL, less than 8 mcg/1 mL, less than 5 mcg/1 mL, less than 2.5 mcg/1 mL, less than 2.125 mcg/1 mL, less than 1.25 mcg/1 mL, or the therapeutic unit dose volume may comprise tiotropium at a concentration of less than 0.625 mcg/1 mL. In certain embodiments, for example, the tiotropium solution may be provided in a therapeutic unit dose volume of 1 mL, the therapeutic unit dose volume comprising tiotropium at a concentration of 5 mcg/1 mL, 5.1 mcg/1 mL, 5.2 mcg/1 mL, 5.3 mcg/1 mL, 5.4 mcg/1 mL, 5.5 mcg/1 mL, 5.6 mcg/1 mL, 5.7 mcg/1 mL, 5.8 mcg/1 mL, 5.9 mcg/1 mL, 6 mcg/1 mL, 6.1 mcg/1 mL, 6.2 mcg/1 mL, 6.3 mcg/1 mL, 6.4 mcg/1 mL, 6.5 mcg/1 mL, 6.6 mcg/1 mL, 6.7 mcg/1 mL, 6.8 mcg/1 mL, 6.9 mcg/1 mL, 7 mcg/1 mL, 7.1 mcg/1 mL, 7.2 mcg/1 mL, 7.3 mcg/1 mL, 7.4 mcg/1 mL, 7.5 mcg/1 mL, 7.6 mcg/1 mL, 7.7 mcg/1 mL, 7.8 mcg/1 mL, 7.9 mcg/1 mL, 8 mcg/1 mL, 8.1 mcg/1 mL, 8.2 mcg/1 mL, 8.3 mcg/1 mL, 8.4 mcg/1 mL, 8.5 mcg/1 mL, 8.6 mcg/1 mL, 8.7 mcg/1 mL, 8.8 mcg/1 mL, 8.9 mcg/1 mL, 9 mcg/1 mL, 9.1 mcg/1 mL, 9.2 mcg/1 mL, 9.3 mcg/1 mL, 9.4 mcg/1 mL, 9.5 mcg/1 mL, 9.6 mcg/1 mL, 9.7 mcg/1 mL, 9.8 mcg/1 mL, 9.9 mcg/1 mL, 10 mcg/1 mL, 10.1 mcg/1 mL, 10.2 mcg/1 mL, 10.3 mcg/1 mL, 10.4 mcg/1 mL, 10.5 mcg/1 mL, 10.6 mcg/1 mL, 10.7 mcg/1 mL, 10.8 mcg/1 mL, 10.9 mcg/1 mL, 11 mcg/1 mL, 11.1 mcg/1 mL, 11.2 mcg/1 mL, 11.3 mcg/1 mL, 11.4 mcg/1 mL, 11.5 mcg/1 mL, 11.6 mcg/1 mL, 11.7 mcg/1 mL, 11.8 mcg/1 mL, 11.9 mcg/1 mL, 12 mcg/1 mL, 12.1 mcg/1 mL, 12.2 mcg/1 mL, 12.3 mcg/1 mL, 12.4 mcg/1 mL, 12.5 mcg/1 mL, 12.6 mcg/1 mL, 12.7 mcg/1 mL, 12.8 mcg/1 mL, 12.9 mcg/1 mL, 13 mcg/1 mL, 13.1 mcg/1 mL, 13.2 mcg/1 mL, 13.3 mcg/1 mL, 13.4 mcg/1 mL, 13.5 mcg/1 mL, 13.6 mcg/1 mL, 13.7 mcg/1 mL, 13.8 mcg/1 mL, 13.9 mcg/1 mL, 14 mcg/1 mL, 14.1 mcg/1 mL, 14.2 mcg/1 mL, 14.3 mcg/1 mL, 14.4 mcg/1 mL, 14.5 mcg/1 mL, 14.6 mcg/1 mL, 14.7 mcg/1 mL, 14.8 mcg/1 mL, 14.9 mcg/1 mL, 15 mcg/1 mL, 15.1 mcg/1 mL, 15.2 mcg/1 mL, 15.3 mcg/1 mL, 15.4 mcg/1 mL, 15.5 mcg/1 mL, 15.6 mcg/1 mL, 15.7 mcg/1 mL, 15.8 mcg/1 mL, 15.9 mcg/1 mL, 16 mcg/1 mL, 16.1 mcg/1 mL, 16.2 mcg/1 mL, 16.3 mcg/1 mL, 16.4 mcg/1 mL, 16.5 mcg/1 mL, 16.6 mcg/1 mL, 16.7 mcg/1 mL, 16.8 mcg/1 mL, 16.9 mcg/1 mL, 17 mcg/1 mL, 17.1 mcg/1 mL, 17.2 mcg/1 mL, 17.3 mcg/1 mL, 17.4 mcg/1 mL, 17.5 mcg/1 mL, 17.6 mcg/1 mL, 17.7 mcg/1 mL, 17.8 mcg/1 mL, 17.9 mcg/1 mL, 18 mcg/1 mL, 18.1 mcg/1 mL, 18.2 mcg/1 mL, 18.3 mcg/1 mL, 18.4 mcg/1 mL, 18.5 mcg/1 mL, 18.6 mcg/1 mL, 18.7 mcg/1 mL, 18.8 mcg/1 mL, 18.9 mcg/1 mL, 19 mcg/1 mL, 19.1 mcg/1 mL, 19.2 mcg/1 mL, 19.3 mcg/1 mL, 19.4 mcg/1 mL, 19.5 mcg/1 mL, 19.6 mcg/1 mL, 19.7 mcg/1 mL, 19.8 mcg/1 mL, 19.9 mcg/1 mL, or tiotropium at a concentration of 20 mcg/1 mL.

In certain embodiments, for example, the tiotropium solution may be provided in a therapeutic unit dose volume of 1.25 mL, the therapeutic unit dose volume comprising tiotropium at a concentration in the range of 0.25-10 mcg/1.25 mL, for example in the range of 0.25-0.5 mcg/1.25 mL, in the range of 0.5-0.75 mcg/1.25 mL, in the range of 0.75-1 mcg/1.25 mL, in the range of 1-1.25 mcg/1.25 mL, in the range of 1.25-2 mcg/1.25 mL, in the range of 2-2.25 mcg/1.25 mL, in the range of 2.25-2.5 mcg/1.25 mL, in the range of 2.5-2.625 mcg/1.25 mL, in the range of 2.625-3 mcg/1.25 mL, in the range of 3-5 mcg/1.25 mL, in the range of 5-8 mcg/1.25 mL, or the therapeutic unit dose volume may comprise tiotropium at a concentration in the range of 8-10 mcg/1.25 mL. In certain embodiments, for example, the tiotropium solution may be provided in a therapeutic unit dose volume of 1.25 mL, the therapeutic unit dose volume comprising tiotropium at a concentration of less than 10 mcg/1.25 mL, less than 8 mcg/1.25 mL, less than 5 mcg/1.25 mL, less than 2.5 mcg/1.25 mL, less than 2.125 mcg/1.25 mL, less than 1.25 mcg/1.25 mL, or the therapeutic unit dose volume may comprise tiotropium at a concentration of less than 0.625 mcg/1.25 mL. In certain embodiments, for example, the tiotropium solution may be provided in a therapeutic unit dose volume of 1.25 mL, the therapeutic unit dose volume comprising tiotropium at a concentration of 5 mcg/1.25 mL, 5.1 mcg/1.25 mL, 5.2 mcg/1.25 mL, 5.3 mcg/1.25 mL, 5.4 mcg/1.25 mL, 5.5 mcg/1.25 mL, 5.6 mcg/1.25 mL, 5.7 mcg/1.25 mL, 5.8 mcg/1.25 mL, 5.9 mcg/1.25 mL, 6 mcg/1.25 mL, 6.1 mcg/1.25 mL, 6.2 mcg/1.25 mL, 6.3 mcg/1.25 mL, 6.4 mcg/1.25 mL, 6.5 mcg/1.25 mL, 6.6 mcg/1.25 mL, 6.7 mcg/1.25 mL, 6.8 mcg/1.25 mL, 6.9 mcg/1.25 mL, 7 mcg/1.25 mL, 7.1 mcg/1.25 mL, 7.2 mcg/1.25 mL, 7.3 mcg/1.25 mL, 7.4 mcg/1.25 mL, 7.5 mcg/1.25 mL, 7.6 mcg/1.25 mL, 7.7 mcg/1.25 mL, 7.8 mcg/1.25 mL, 7.9 mcg/1.25 mL, 8 mcg/1.25 mL, 8.1 mcg/1.25 mL, 8.2 mcg/1.25 mL, 8.3 mcg/1.25 mL, 8.4 mcg/1.25 mL, 8.5 mcg/1.25 mL, 8.6 mcg/1.25 mL, 8.7 mcg/1.25 mL, 8.8 mcg/1.25 mL, 8.9 mcg/1.25 mL, 9 mcg/1.25 mL, 9.1 mcg/1.25 mL, 9.2 mcg/1.25 mL, 9.3 mcg/1.25 mL, 9.4 mcg/1.25 mL, 9.5 mcg/1.25 mL, 9.6 mcg/1.25 mL, 9.7 mcg/1.25 mL, 9.8 mcg/1.25 mL, 9.9 mcg/1.25 mL, 10 mcg/1.25 mL, 10.1 mcg/1.25 mL, 10.2 mcg/1.25 mL, 10.3 mcg/1.25 mL, 10.4 mcg/1.25 mL, 10.5 mcg/1.25 mL, 10.6 mcg/1.25 mL, 10.7 mcg/1.25 mL, 10.8 mcg/1.25 mL, 10.9 mcg/1.25 mL, 11 mcg/1.25 mL, 11.1 mcg/1.25 mL, 11.2 mcg/1.25 mL, 11.3 mcg/1.25 mL, 11.4 mcg/1.25 mL, 11.5 mcg/1.25 mL, 11.6 mcg/1.25 mL, 11.7 mcg/1.25 mL, 11.8 mcg/1.25 mL, 11.9 mcg/1.25 mL, 12 mcg/1.25 mL, 12.1 mcg/1.25 mL, 12.2 mcg/1.25 mL, 12.3 mcg/1.25 mL, 12.4 mcg/1.25 mL, 12.5 mcg/1.25 mL, 12.6 mcg/1.25 mL, 12.7 mcg/1.25 mL, 12.8 mcg/1.25 mL, 12.9 mcg/1.25 mL, 13 mcg/1.25 mL, 13.1 mcg/1.25 mL, 13.2 mcg/1.25 mL, 13.3 mcg/1.25 mL, 13.4 mcg/1.25 mL, 13.5 mcg/1.25 mL, 13.6 mcg/1.25 mL, 13.7 mcg/1.25 mL, 13.8 mcg/1.25 mL, 13.9 mcg/1.25 mL, 14 mcg/1.25 mL, 14.1 mcg/1.25 mL, 14.2 mcg/1.25 mL, 14.3 mcg/1.25 mL, 14.4 mcg/1.25 mL, 14.5 mcg/1.25 mL, 14.6 mcg/1.25 mL, 14.7 mcg/1.25 mL, 14.8 mcg/1.25 mL, 14.9 mcg/1.25 mL, 15 mcg/1.25 mL, 15.1 mcg/1.25 mL, 15.2 mcg/1.25 mL, 15.3 mcg/1.25 mL, 15.4 mcg/1.25 mL, 15.5 mcg/1.25 mL, 15.6 mcg/1.25 mL, 15.7 mcg/1.25 mL, 15.8 mcg/1.25 mL, 15.9 mcg/1.25 mL, 16 mcg/1.25 mL, 16.1 mcg/1.25 mL, 16.2 mcg/1.25 mL, 16.3 mcg/1.25 mL, 16.4 mcg/1.25 mL, 16.5 mcg/1.25 mL, 16.6 mcg/1.25 mL, 16.7 mcg/1.25 mL, 16.8 mcg/1.25 mL, 16.9 mcg/1.25 mL, 17 mcg/1.25 mL, 17.1 mcg/1.25 mL, 17.2 mcg/1.25 mL, 17.3 mcg/1.25 mL, 17.4 mcg/1.25 mL, 17.5 mcg/1.25 mL, 17.6 mcg/1.25 mL, 17.7 mcg/1.25 mL, 17.8 mcg/1.25 mL, 17.9 mcg/1.25 mL, 18 mcg/1.25 mL, 18.1 mcg/1.25 mL, 18.2 mcg/1.25 mL, 18.3 mcg/1.25 mL, 18.4 mcg/1.25 mL, 18.5 mcg/1.25 mL, 18.6 mcg/1.25 mL, 18.7 mcg/1.25 mL, 18.8 mcg/1.25 mL, 18.9 mcg/1.25 mL, 19 mcg/1.25 mL, 19.1 mcg/1.25 mL, 19.2 mcg/1.25 mL, 19.3 mcg/1.25 mL, 19.4 mcg/1.25 mL, 19.5 mcg/1.25 mL, 19.6 mcg/1.25 mL, 19.7 mcg/1.25 mL, 19.8 mcg/1.25 mL, 19.9 mcg/1.25 mL, or tiotropium at a concentration of 20 mcg/1.25 mL.

In certain embodiments, for example, the tiotropium solution may be provided in a therapeutic unit dose volume of 1.5 mL, the therapeutic unit dose volume comprising tiotropium at a concentration in the range of 0.25-10 mcg/1.5 mL, for example in the range of 0.25-0.5 mcg/1.5 mL, in the range of 0.5-0.75 mcg/1.5 mL, in the range of 0.75-1 mcg/1.5 mL, in the range of 1-1.25 mcg/1.5 mL, in the range of 1.25-2 mcg/1.5 mL, in the range of 2-2.25 mcg/1.5 mL, in the range of 2.25-2.5 mcg/1.5 mL, in the range of 2.5-2.625 mcg/1.5 mL, in the range of 2.625-3 mcg/1.5 mL, in the range of 3-5 mcg/1.5 mL, in the range of 5-8 mcg/1.5 mL, or the therapeutic unit dose volume may comprise tiotropium at a concentration in the range of 8-10 mcg/1.5 mL. In certain embodiments, for example, the tiotropium solution may be provided in a therapeutic unit dose volume of 1.5 mL, the therapeutic unit dose volume comprising tiotropium at a concentration of less than 10 mcg/1.5 mL, less than 8 mcg/1.5 mL, less than 5 mcg/1.5 mL, less than 2.5 mcg/1.5 mL, less than 2.125 mcg/1.5 mL, less than 1.25 mcg/1.5 mL, or the therapeutic unit dose volume may comprise tiotropium at a concentration of less than 0.625 mcg/1.5 mL. In certain embodiments, for example, the tiotropium solution may be provided in a therapeutic unit dose volume of 1.5 mL, the therapeutic unit dose volume comprising tiotropium at a concentration of 5 mcg/1.5 mL, 5.1 mcg/1.5 mL, 5.2 mcg/1.5 mL, 5.3 mcg/1.5 mL, 5.4 mcg/1.5 mL, 5.5 mcg/1.5 mL, 5.6 mcg/1.5 mL, 5.7 mcg/1.5 mL, 5.8 mcg/1.5 mL, 5.9 mcg/1.5 mL, 6 mcg/1.5 mL, 6.1 mcg/1.5 mL, 6.2 mcg/1.5 mL, 6.3 mcg/1.5 mL, 6.4 mcg/1.5 mL, 6.5 mcg/1.5 mL, 6.6 mcg/1.5 mL, 6.7 mcg/1.5 mL, 6.8 mcg/1.5 mL, 6.9 mcg/1.5 mL, 7 mcg/1.5 mL, 7.1 mcg/1.5 mL, 7.2 mcg/1.5 mL, 7.3 mcg/1.5 mL, 7.4 mcg/1.5 mL, 7.5 mcg/1.5 mL, 7.6 mcg/1.5 mL, 7.7 mcg/1.5 mL, 7.8 mcg/1.5 mL, 7.9 mcg/1.5 mL, 8 mcg/1.5 mL, 8.1 mcg/1.5 mL, 8.2 mcg/1.5 mL, 8.3 mcg/1.5 mL, 8.4 mcg/1.5 mL, 8.5 mcg/1.5 mL, 8.6 mcg/1.5 mL, 8.7 mcg/1.5 mL, 8.8 mcg/1.5 mL, 8.9 mcg/1.5 mL, 9 mcg/1.5 mL, 9.1 mcg/1.5 mL, 9.2 mcg/1.5 mL, 9.3 mcg/1.5 mL, 9.4 mcg/1.5 mL, 9.5 mcg/1.5 mL, 9.6 mcg/1.5 mL, 9.7 mcg/1.5 mL, 9.8 mcg/1.5 mL, 9.9 mcg/1.5 mL, 10 mcg/1.5 mL, 10.1 mcg/1.5 mL, 10.2 mcg/1.5 mL, 10.3 mcg/1.5 mL, 10.4 mcg/1.5 mL, 10.5 mcg/1.5 mL, 10.6 mcg/1.5 mL, 10.7 mcg/1.5 mL, 10.8 mcg/1.5 mL, 10.9 mcg/1.5 mL, 11 mcg/1.5 mL, 11.1 mcg/1.5 mL, 11.2 mcg/1.5 mL, 11.3 mcg/1.5 mL, 11.4 mcg/1.5 mL, 11.5 mcg/1.5 mL, 11.6 mcg/1.5 mL, 11.7 mcg/1.5 mL, 11.8 mcg/1.5 mL, 11.9 mcg/1.5 mL, 12 mcg/1.5 mL, 12.1 mcg/1.5 mL, 12.2 mcg/1.5 mL, 12.3 mcg/1.5 mL, 12.4 mcg/1.5 mL, 12.5 mcg/1.5 mL, 12.6 mcg/1.5 mL, 12.7 mcg/1.5 mL, 12.8 mcg/1.5 mL, 12.9 mcg/1.5 mL, 13 mcg/1.5 mL, 13.1 mcg/1.5 mL, 13.2 mcg/1.5 mL, 13.3 mcg/1.5 mL, 13.4 mcg/1.5 mL, 13.5 mcg/1.5 mL, 13.6 mcg/1.5 mL, 13.7 mcg/1.5 mL, 13.8 mcg/1.5 mL, 13.9 mcg/1.5 mL, 14 mcg/1.5 mL, 14.1 mcg/1.5 mL, 14.2 mcg/1.5 mL, 14.3 mcg/1.5 mL, 14.4 mcg/1.5 mL, 14.5 mcg/1.5 mL, 14.6 mcg/1.5 mL, 14.7 mcg/1.5 mL, 14.8 mcg/1.5 mL, 14.9 mcg/1.5 mL, 15 mcg/1.5 mL, 15.1 mcg/1.5 mL, 15.2 mcg/1.5 mL, 15.3 mcg/1.5 mL, 15.4 mcg/1.5 mL, 15.5 mcg/1.5 mL, 15.6 mcg/1.5 mL, 15.7 mcg/1.5 mL, 15.8 mcg/1.5 mL, 15.9 mcg/1.5 mL, 16 mcg/1.5 mL, 16.1 mcg/1.5 mL, 16.2 mcg/1.5 mL, 16.3 mcg/1.5 mL, 16.4 mcg/1.5 mL, 16.5 mcg/1.5 mL, 16.6 mcg/1.5 mL, 16.7 mcg/1.5 mL, 16.8 mcg/1.5 mL, 16.9 mcg/1.5 mL, 17 mcg/1.5 mL, 17.1 mcg/1.5 mL, 17.2 mcg/1.5 mL, 17.3 mcg/1.5 mL, 17.4 mcg/1.5 mL, 17.5 mcg/1.5 mL, 17.6 mcg/1.5 mL, 17.7 mcg/1.5 mL, 17.8 mcg/1.5 mL, 17.9 mcg/1.5 mL, 18 mcg/1.5 mL, 18.1 mcg/1.5 mL, 18.2 mcg/1.5 mL, 18.3 mcg/1.5 mL, 18.4 mcg/1.5 mL, 18.5 mcg/1.5 mL, 18.6 mcg/1.5 mL, 18.7 mcg/1.5 mL, 18.8 mcg/1.5 mL, 18.9 mcg/1.5 mL, 19 mcg/1.5 mL, 19.1 mcg/1.5 mL, 19.2 mcg/1.5 mL, 19.3 mcg/1.5 mL, 19.4 mcg/1.5 mL, 19.5 mcg/1.5 mL, 19.6 mcg/1.5 mL, 19.7 mcg/1.5 mL, 19.8 mcg/1.5 mL, 19.9 mcg/1.5 mL, or tiotropium at a concentration of 20 mcg/1.5 mL.

In certain embodiments, for example, the tiotropium solution may be provided in a therapeutic unit dose volume of 2 mL, the therapeutic unit dose volume comprising tiotropium at a concentration in the range of 0.25-10 mcg/2 mL, for example in the range of 0.25-0.5 mcg/2 mL, in the range of 0.5-0.75 mcg/2 mL, in the range of 0.75-1 mcg/2 mL, in the range of 1-1.25 mcg/2 mL, in the range of 1.25-2 mcg/2 mL, in the range of 2-2.25 mcg/2 mL, in the range of 2.25-2.5 mcg/2 mL, in the range of 2.5-2.625 mcg/2 mL, in the range of 2.625-3 mcg/2 mL, in the range of 3-5 mcg/2 mL, in the range of 5-8 mcg/2 mL, or the therapeutic unit dose volume may comprise tiotropium at a concentration in the range of 8-10 mcg/2 mL. In certain embodiments, for example, the tiotropium solution may be provided in a therapeutic unit dose volume of 2 mL, the therapeutic unit dose volume comprising tiotropium at a concentration of less than 10 mcg/2 mL, less than 8 mcg/2 mL, less than 5 mcg/2 mL, less than 2.5 mcg/2 mL, less than 2.125 mcg/2 mL, less than 1.25 mcg/2 mL, or the therapeutic unit dose volume may comprise tiotropium at a concentration of less than 0.625 mcg/2 mL. In certain embodiments, for example, the tiotropium solution may be provided in a therapeutic unit dose volume of 2 mL, the therapeutic unit dose volume comprising tiotropium at a concentration of 5 mcg/2 mL, 5.1 mcg/2 mL, 5.2 mcg/2 mL, 5.3 mcg/2 mL, 5.4 mcg/2 mL, 5.5 mcg/2 mL, 5.6 mcg/2 mL, 5.7 mcg/2 mL, 5.8 mcg/2 mL, 5.9 mcg/2 mL, 6 mcg/2 mL, 6.1 mcg/2 mL, 6.2 mcg/2 mL, 6.3 mcg/2 mL, 6.4 mcg/2 mL, 6.5 mcg/2 mL, 6.6 mcg/2 mL, 6.7 mcg/2 mL, 6.8 mcg/2 mL, 6.9 mcg/2 mL, 7 mcg/2 mL, 7.1 mcg/2 mL, 7.2 mcg/2 mL, 7.3 mcg/2 mL, 7.4 mcg/2 mL, 7.5 mcg/2 mL, 7.6 mcg/2 mL, 7.7 mcg/2 mL, 7.8 mcg/2 mL, 7.9 mcg/2 mL, 8 mcg/2 mL, 8.1 mcg/2 mL, 8.2 mcg/2 mL, 8.3 mcg/2 mL, 8.4 mcg/2 mL, 8.5 mcg/2 mL, 8.6 mcg/2 mL, 8.7 mcg/2 mL, 8.8 mcg/2 mL, 8.9 mcg/2 mL, 9 mcg/2 mL, 9.1 mcg/2 mL, 9.2 mcg/2 mL, 9.3 mcg/2 mL, 9.4 mcg/2 mL, 9.5 mcg/2 mL, 9.6 mcg/2 mL, 9.7 mcg/2 mL, 9.8 mcg/2 mL, 9.9 mcg/2 mL, 10 mcg/2 mL, 10.1 mcg/2 mL, 10.2 mcg/2 mL, 10.3 mcg/2 mL, 10.4 mcg/2 mL, 10.5 mcg/2 mL, 10.6 mcg/2 mL, 10.7 mcg/2 mL, 10.8 mcg/2 mL, 10.9 mcg/2 mL, 11 mcg/2 mL, 11.1 mcg/2 mL, 11.2 mcg/2 mL, 11.3 mcg/2 mL, 11.4 mcg/2 mL, 11.5 mcg/2 mL, 11.6 mcg/2 mL, 11.7 mcg/2 mL, 11.8 mcg/2 mL, 11.9 mcg/2 mL, 12 mcg/2 mL, 12.1 mcg/2 mL, 12.2 mcg/2 mL, 12.3 mcg/2 mL, 12.4 mcg/2 mL, 12.5 mcg/2 mL, 12.6 mcg/2 mL, 12.7 mcg/2 mL, 12.8 mcg/2 mL, 12.9 mcg/2 mL, 13 mcg/2 mL, 13.1 mcg/2 mL, 13.2 mcg/2 mL, 13.3 mcg/2 mL, 13.4 mcg/2 mL, 13.5 mcg/2 mL, 13.6 mcg/2 mL, 13.7 mcg/2 mL, 13.8 mcg/2 mL, 13.9 mcg/2 mL, 14 mcg/2 mL, 14.1 mcg/2 mL, 14.2 mcg/2 mL, 14.3 mcg/2 mL, 14.4 mcg/2 mL, 14.5 mcg/2 mL, 14.6 mcg/2 mL, 14.7 mcg/2 mL, 14.8 mcg/2 mL, 14.9 mcg/2 mL, 15 mcg/2 mL, 15.1 mcg/2 mL, 15.2 mcg/2 mL, 15.3 mcg/2 mL, 15.4 mcg/2 mL, 15.5 mcg/2 mL, 15.6 mcg/2 mL, 15.7 mcg/2 mL, 15.8 mcg/2 mL, 15.9 mcg/2 mL, 16 mcg/2 mL, 16.1 mcg/2 mL, 16.2 mcg/2 mL, 16.3 mcg/2 mL, 16.4 mcg/2 mL, 16.5 mcg/2 mL, 16.6 mcg/2 mL, 16.7 mcg/2 mL, 16.8 mcg/2 mL, 16.9 mcg/2 mL, 17 mcg/2 mL, 17.1 mcg/2 mL, 17.2 mcg/2 mL, 17.3 mcg/2 mL, 17.4 mcg/2 mL, 17.5 mcg/2 mL, 17.6 mcg/2 mL, 17.7 mcg/2 mL, 17.8 mcg/2 mL, 17.9 mcg/2 mL, 18 mcg/2 mL, 18.1 mcg/2 mL, 18.2 mcg/2 mL, 18.3 mcg/2 mL, 18.4 mcg/2 mL, 18.5 mcg/2 mL, 18.6 mcg/2 mL, 18.7 mcg/2 mL, 18.8 mcg/2 mL, 18.9 mcg/2 mL, 19 mcg/2 mL, 19.1 mcg/2 mL, 19.2 mcg/2 mL, 19.3 mcg/2 mL, 19.4 mcg/2 mL, 19.5 mcg/2 mL, 19.6 mcg/2 mL, 19.7 mcg/2 mL, 19.8 mcg/2 mL, 19.9 mcg/2 mL, or tiotropium at a concentration of 20 mcg/2 mL.

In certain embodiments, for example, the tiotropium solution may comprise added tiotropium bromide. In certain embodiments, for example, the tiotropium solution may comprise added tiotropium bromide monohydrate. In certain embodiments, for example, the tiotropium solution may comprise an added crystalline tiotropium compound (for example the added tiotropium compound may comprise at least 25 wt. % crystalline tiotropium bromide, relative to the total weight of the tiotropium compound added, or the added tiotropium compound may comprise at least 25 wt. % crystalline tiotropium bromide monohydrate, relative to the total weight of the tiotropium compound added). In certain embodiments, for example, the tiotropium solution may comprise an added amorphous tiotropium compound (for example the added tiotropium compound may be at least 90 wt. % amorphous relative to the total weight of the tiotropium compound). In certain embodiments, for example, the tiotropium solution may comprise an added anhydrous tiotropium compound (for example the added tiotropium compound may contain less than 0.1 wt. % occluded and/or co-crystalline water relative to the total weight of the tiotropium compound). In certain embodiments, for example, the tiotropium solution may comprise an added anhydrous amorphous tiotropium compound.

In certain embodiments, for example, the tiotropium solution may comprise at least a second drug. In certain embodiments, for example, the at least a second drug may be an active ingredient for treatment of an inflammatory lung disease. In certain further embodiments, for example, the at least a second drug may comprise a therapeutically effective dose of olodaterol hydrochloride. In certain further embodiments, for example, the therapeutically effective unit dose of olodaterol hydrochloride may be in the range of 0.25 mcg to no more than 10 mcg, for example in the range of 0.25 mcg to no more than 0.5 mcg, in the range of 0.5 mcg to no more than 0.75 mcg, in the range of 0.75 mcg to no more than 1 mcg, in the range of 1 mcg to no more than 1.25 mcg, in the range of 1.25 mcg to no more than 2 mcg, in the range of 2 mcg to no more than 2.25 mcg, in the range of 2.25 mcg to no more than 2.5 mcg, in the range of 2.5 mcg to no more than 2.625 mcg, in the range of 2.625 mcg to no more than 3 mcg, in the range of 3 mcg to no more than 5 mcg, in the range of 5 mcg to no more than 8 mcg, or the therapeutically effective unit dose of olodaterol hydrochloride may be in the range of 8 mcg to no more than 10 mcg. In certain embodiments, for example, the therapeutically effective unit dose of olodaterol hydrochloride may be less than 10 mcg, less than 8 mcg, less than 5 mcg, less than 2.5 mcg, less than 2.125 mcg, less than 1.25 mcg, or the therapeutically effective unit dose of olodaterol hydrochloride may be less than 0.625 mcg. In certain embodiments, for example, the tiotropium solution may comprise olodaterol hydrochloride at a concentration in the range of 0.000025-0.001 wt. %, for example in the range of 0.000025-0.00005 wt. %, in the range of 0.00005-0.000075 wt. %, in the range of 0.000075-0.0001 wt. %, in the range of 0.0001-0.000125 wt. %, in the range of 0.000125-0.0002 wt. %, in the range of 0.0002-0.000225 wt. %, in the range of 0.000225-0.00025 wt. %, in the range of 0.00025-0.0002625 wt. %, in the range of 0.0002625-0.0003 wt. %, in the range of 0.0003-0.0005 wt. %, in the range of 0.0005-0.0008 wt. %, or the tiotropium solution may comprise olodaterol hydrochloride at a concentration in the range of 0.0008-0.001 wt. %. In certain embodiments, for example, the tiotropium solution may comprise olodaterol hydrochloride at a concentration of less than 0.001 wt. %, less than 0.0008 wt. %, less than 0.0005 wt. %, less than 0.00025 wt. %, less than 0.0002125 wt. %, less than 0.000126 wt. %, or the tiotropium solution may comprise olodaterol hydrochloride at a concentration of less than 0.0000625 wt. %. In certain embodiments, for example, the tiotropium solution may be provided in a therapeutic unit dose volume of 1 mL, the therapeutic unit dose volume comprising olodaterol hydrochloride at a concentration in the range of 0.25-10 mcg/mL, for example in the range of 0.25-0.5 mcg/mL, in the range of 0.5-0.75 mcg/mL, in the range of 0.75-1 mcg/mL, in the range of 1-1.25 mcg/mL, in the range of 1.25-2 mcg/mL, in the range of 2-2.25 mcg/mL, in the range of 2.25-2.5 mcg/mL, in the range of 2.5-2.625 mcg/mL, in the range of 2.625-3 mcg/mL, in the range of 3-5 mcg/mL, in the range of 5-8 mcg/mL, or the therapeutic unit dose volume may comprise olodaterol hydrochloride at a concentration in the range of 8-10 mcg/mL. In certain embodiments, for example, the tiotropium solution may be provided in a therapeutic unit dose volume of 1 mL, the therapeutic unit dose volume comprising olodaterol hydrochloride at a concentration of less than 10 mcg/mL, less than 8 mcg/mL, less than 5 mcg/mL, less than 2.5 mcg/mL, less than 2.125 mcg/mL, less than 1.25 mcg/mL, or the therapeutic unit dose volume may comprise olodaterol hydrochloride at a concentration of less than 0.625 mcg/mL. In certain embodiments, for example, the tiotropium solution may be provided in a therapeutic unit dose volume of 2 mL, the therapeutic unit dose volume comprising olodaterol hydrochloride at a concentration in the range of 0.25-10 mcg/2 mL, for example in the range of 0.25-0.5 mcg/2 mL, in the range of 0.5-0.75 mcg/2 mL, in the range of 0.75-1 mcg/2 mL, in the range of 1-1.25 mcg/2 mL, in the range of 1.25-2 mcg/2 mL, in the range of 2-2.25 mcg/2 mL, in the range of 2.25-2.5 mcg/2 mL, in the range of 2.5-2.625 mcg/2 mL, in the range of 2.625-3 mcg/2 mL, in the range of 3-5 mcg/2 mL, in the range of 5-8 mcg/2 mL, or the therapeutic unit dose volume may comprise olodaterol hydrochloride at a concentration in the range of 8-10 mcg/2 mL. In certain embodiments, for example, the tiotropium solution may be provided in a therapeutic unit dose volume of 2 mL, the therapeutic unit dose volume comprising olodaterol hydrochloride at a concentration of less than 10 mcg/2 mL, less than 8 mcg/2 mL, less than 5 mcg/2 mL, less than 2.5 mcg/2 mL, less than 2.125 mcg/2 mL, less than 1.25 mcg/2 mL, or the therapeutic unit dose volume may comprise olodaterol hydrochloride at a concentration of less than 0.625 mcg/2 mL.

In certain embodiments, for example, the tiotropium solution may comprise water. In certain embodiments, for example, the tiotropium solution may comprise less than 99 wt. % water, for example less than 98 wt. %, less than 97.5 wt. % or the tiotropium solution may comprise less than 97 wt. % water. In certain embodiments, for example, the tiotropium solution may comprise in the range of 95-99 wt. % water, for example in the range of 97-99 wt. %, or the tiotropium solution may comprise in the range of 97-98 wt. % water. In certain embodiments, for example, the tiotropium solution may comprise at least 97 wt. % water. In certain embodiments, for example, the tiotropium solution may comprise in the range of 60-99 wt. % water, for example in the range of 60-70 wt. % water, in the range of 70-80 wt. % water, in the range of 80-90 wt. % water, or the tiotropium solution may comprise in the range of 90-99 wt. % water. In certain embodiments, for example, the tiotropium solution may comprise a quantum satis amount (“q.s.”) sufficient to bring a concentration of tiotropium in the solution to an indicated concentration.

In certain embodiments, for example, the tiotropium solution may comprise one or plural cosolvents. In certain further embodiments, for example, the one or plural cosolvents may be selected from the group consisting of alcohols, ethers, hydrocarbons, perfluorocarbons, and a combination of two of more of the foregoing cosolvents. In certain embodiments, for example, the one or plural cosolvents may comprise a short chain polar alcohol. In certain embodiments, for example, the one or plural cosolvents may comprise an aliphatic alcohol having from one to six carbon atoms, such as methanol, ethanol or propanol (for example isopropanol). In certain embodiments, for example, the one or plural cosolvents may comprise ethanol (for example the one or plural cosolvents may be ethanol). In certain embodiments, for example, the one or plural cosolvents may comprise a hydrocarbon selected from the group consisting of n-butane, isobutane, pentane, neopentane, isopentane, and a combination of two or more of the foregoing hydrocarbons. In certain embodiments, for example, the one or plural cosolvents may comprise an ether selected from the group consisting of dimethyl ether, diethyl ether, and a combination of the two ethers. In certain embodiments, for example, the one or plural cosolvents may comprise a perfluorocarbon selected from the group consisting of perfluoropropane, perfluorobutane, perfluorocyclobutane, perfluoropentane, and a combination of two or more of the foregoing perfluorocarbons.

In certain embodiments, for example, the cosolvent may comprise ethanol at a concentration in the tiotropium solution in the range of 0.5-40 wt. %, for example at a concentration in the range of 0.5-10 wt. %, in the range of 10-20 wt. %, in the range of 20-30 wt. %, or the ethanol may be present in the tiotropium solution at a concentration in the range of 30-40 wt. %. In certain embodiments, for example, the tiotropium solution may comprise an ethanol concentration of at least 5 wt. %, at least 10 wt. %, at least 25 wt. %, or the tiotropium solution may comprise an ethanol concentration of at least 35 wt. %. In certain embodiments, for example, the tiotropium solution may comprise an ethanol concentration of less than 25 wt. %, for example an ethanol concentration of less than 10 wt. %, or the tiotropium solution may comprise an ethanol concentration of less than 5 wt. %.

In certain embodiments, for example, the tiotropium solution may comprise citric acid at a concentration in the range of 0.005-0.2 wt. %, for example at a concentration in the range of 0.01-0.15 wt. %, in the range of 0.015-0.1 wt. %, in the range of 0.025-0.075 wt. %, in the range of 0.04-0.06 wt. %, in the range of 0.06-0.08 wt. %, or the tiotropium solution may comprise citric acid at a concentration in the range of 0.055-0.065 wt. %. In certain embodiments, for example, the tiotropium solution may comprise citric acid at a concentration of less than 0.5 wt. %, for example a concentration of less than 0.25 wt. %, less than 0.1 wt. %, less than 0.075 wt. %, less than 0.06 wt. %, less than 0.03 wt. %, less than 0.02 wt. %, or the tiotropium solution may comprise citric acid at a concentration of less than 0.01 wt. %. In certain embodiments, for example, the tiotropium solution may comprise citric acid at a concentration of at least 0.01 wt. %, for example a concentration of at least 0.02 wt. %, at least 0.03 wt. %, at least 0.04 wt. %, or the tiotropium solution may comprise citric acid at a concentration of at least 0.06 wt. %.

In certain embodiments, for example, the tiotropium solution may comprise sodium chloride at a concentration in the range of 0.01-2 wt. %, for example at a concentration in the range of 0.25-1.5 wt. %, in the range of 0.5-1 wt. %, in the range of 0.6-0.7, in the range of 0.7-0.8 wt. %, or the tiotropium solution may comprise sodium chloride at a concentration in the range of 0.68-0.72 wt. %. In certain embodiments, for example, the tiotropium solution may comprise sodium chloride at a concentration of less than 1 wt. %, for example at a concentration of less than 0.72 wt. %.

In certain embodiments, for example, the tiotropium solution may comprise sodium citrate at a concentration in the range of 0.001-1 wt. %, for example at a concentration in the range of 0.01-0.75 wt. %, in the range of 0.1-0.6 wt. %, in the range of 0.25-0.5 wt. %, in the range of 0.3-0.4 wt. %, in the range of 0.4-0.5 wt. %, or the tiotropium solution may comprise sodium citrate at a concentration in the range of 0.35-0.45 wt. %. In certain embodiments, for example, the tiotropium solution may comprise sodium citrate at a concentration of less than 1 wt. %, for example a concentration of less than 0.75 wt. %, less than 0.6 wt. %, less than 0.5 wt. %, less than 0.45 wt. %, less than 0.4 wt. %, or the tiotropium solution may comprise sodium citrate at a concentration of less than 0.1 wt. %. In certain embodiments, for example, the tiotropium solution may comprise sodium citrate at a concentration of at least 0.1 wt. %, for example a concentration of at least 0.2 wt. %, at least 0.3 wt. %, at least 0.4 wt. %, or the tiotropium solution may comprise sodium citrate at a concentration of at least 0.5 wt. %.

In certain embodiments, for example, the tiotropium solution may be free, or substantially free (i.e., less than 0.008 wt. %), of preservative including, but not limited to, quaternary ammonium preservatives, such as a benzalkonium salt, (e.g., benzalkonium chloride). In certain embodiments, for example, the tiotropium solution may comprise less than 0.1 wt. % preservative (or quaternary ammonium preservative) (such as less than 0.05 wt. %, less than 0.02 wt. %, or less than 0.008 wt. % preservative).

In certain embodiments, for example, the tiotropium solution may be free, or substantially free (i.e., less than 0.008 wt. %), of complexing agent. In certain embodiments, for example, the tiotropium solution may comprise less than about 0.1 wt. % complexing agent (such as less than about 0.05 wt. %, less than about 0.02 wt. %, or less than about 0.008 wt. %), based on the weight of the tiotropium solution. In certain further embodiments, for example, the tiotropium solution may be free of ethylenediaminetetraacetic acid (EDTA). In certain embodiments, for example, the tiotropium solution may comprise EDTA at a concentration in the range of 0.0001-0.02 wt. % EDTA, for example at a concentration in the range of 0.0025-0.0175 wt. %, in the range of 0.0005-0.0015 wt. %, in the range of 0.0075-0.0125 wt. %, or the tiotropium solution may comprise EDTA at a concentration in the range of 0.009-0.012 wt. %. In certain embodiments, for example, the tiotropium solution may comprise EDTA at a concentration in the range of 0.0001-0.01 wt. % EDTA, for example at a concentration in the range of 0.00075-0.0075 wt. %, in the range of 0.001-0.0075 wt. %, in the range of 0.001-0.005 wt. %, or the tiotropium solution may comprise EDTA at a concentration in the range of 0.002-0.003 wt. %. In certain embodiments, for example, the tiotropium solution may comprise EDTA at a concentration of 0.0095 wt. %. In certain embodiments, for example, the tiotropium solution may comprise EDTA at a concentration of 0.003 wt. %. In certain embodiments, for example, the tiotropium solution may comprise EDTA at a concentration of less than 0.02 wt. %, for example at a concentration of less than 0.015 wt. %, less than 0.01 wt. %, a concentration of less than 0.005 wt. %, or the tiotropium solution may comprise EDTA at a concentration of less than 0.003 wt. %. In certain embodiments, for example, the tiotropium solution may be EDTA-free.

In certain embodiments, for example, the tiotropium solution may be free, or substantially free (i.e., less than 0.008 wt. %), of any component (for example any preservative (for example benzalkonium chloride), stabilizing agent, or complexing agent) which causes airway constriction in an ordinary subject when inhaled. In certain embodiments, for example, the tiotropium solution may be free, or substantially free (i.e., less than 0.008 wt. %) of any component (for example any preservative (for example benzalkonium chloride), stabilizing agent, or complexing agent) which reduces (or offsets) the effectiveness of tiotropium ordinary in an ordinary subject when inhaled.

In certain embodiments, for example, the tiotropium solution may comprise buffer. In certain embodiments, for example, the tiotropium solution may comprise 1-99 wt. % buffer. In certain embodiments, for example, the tiotropium solution may comprise less than 99 wt. % buffer, for example less than 95 wt. %, less than 90 wt. %, less than 75 wt. %, less than 70 wt. %, less than 65 wt. %, less than 60 wt. %, less than 55 wt. %, less than 50 wt. %, less than 45 wt. %, less than 40 wt. %, less than 35 wt. %, less than 30 wt. %, less than 25 wt. %, less than 20 wt. %, less than 15 wt. %, less than 10 wt. %, less than 9 wt. %, less than 8 wt. %, less than 7 wt. %, less than 6 wt. %, less than 5 wt. %, less than 4 wt. %, less than 3 wt. %, less than 2 wt. % or the tiotropium solution may comprise less than 1 wt. % buffer. In certain embodiments, for example, the tiotropium solution may comprise in the range of 1-99 wt. % buffer, for example in the range of 1-10 wt. %, in the range of 10-20 wt. %, in the range of 20-30 wt. %, in the range of 30-40 wt. %, in the range of 40-50 wt. %, in the range of 50-60 wt. %, in the range of 60-70 wt. %, in the range of 70-80 wt. %, in the range of 80-90 wt. %, in the range of 90-95 wt. %, or the tiotropium solution may comprise in the range of 95-99 wt. % buffer. In certain embodiments, for example, the tiotropium solution may comprise in the range of 10-40 wt. % buffer, for example in the range of 15-35 wt. %, in the range of 20-30 wt. %, in the range of 25-30 wt. %, or the tiotropium solution may comprise in the range of 26-29 wt. % buffer. In certain embodiments, for example, the tiotropium solution may comprise 28 wt. % buffer, 28.5 wt. % buffer, or 29 wt. % buffer. In certain embodiments, for example, the tiotropium solution may comprise in the range of 80-99 wt. % buffer, for example in the range of 85-99 wt. %, in the range of 90-99 wt. %, in the range of 95-999 wt. %, or the tiotropium solution may comprise in the range of 97-99 wt. % buffer. In certain embodiments, for example, the tiotropium solution may comprise greater than 97 wt. % buffer.

In certain embodiments, one or more than one (including for instance all) of the following embodiments may comprise each of the embodiments or parts thereof. In certain embodiments, for example, the buffer may comprise a complexing agent. In certain embodiments, for example, the buffer may be complexing agent-free. In certain embodiments, for example, the buffer may comprise a preservative. In certain embodiments, for example, the buffer may be preservative-free. In certain embodiments, for example, the buffer may comprise citric acid. In certain embodiments, for example, the buffer may comprise sodium citrate. In certain embodiments, for example, the buffer may comprise sodium chloride.

In certain embodiments, for example, the buffer may comprise water. In certain embodiments, for example, the buffer may comprise less than 99 wt. % water, for example less than 98 wt. %, less than 97.5 wt. % or the buffer may comprise less than 97 wt. % water. In certain embodiments, for example, the buffer may comprise in the range of 95-99 wt. % water, for example in the range of 97-99 wt. %, or the buffer may comprise in the range of 97-98 wt. % water. In certain embodiments, for example, the buffer may comprise at least 97 wt. % water. In certain embodiments, for example, the buffer may comprise in the range of 60-99 wt. % water, for example in the range of 60-70 wt. % water, in the range of 70-80 wt. % water, in the range of 80-90 wt. % water, or the buffer may comprise in the range of 90-99 wt. % water. In certain embodiments, for example, the buffer may comprise a quantum satis amount (“q.s.”) sufficient to bring a concentration of tiotropium in the solution to an indicated concentration.

In certain embodiments, for example, the buffer may comprise one or plural cosolvents. In certain further embodiments, for example, the one or plural cosolvents may be selected from the group consisting of alcohols, ethers, hydrocarbons, perfluorocarbons, and a combination of two of more of the foregoing cosolvents. In certain embodiments, for example, the one or plural cosolvents may comprise a short chain polar alcohol. In certain embodiments, for example, the one or plural cosolvents may comprise an aliphatic alcohol having from one to six carbon atoms, such as methanol, ethanol or propanol (for example isopropanol). In certain embodiments, for example, the one or plural cosolvents may comprise ethanol (for example the one or plural cosolvents may be ethanol). In certain embodiments, for example, the one or plural cosolvents may comprise a hydrocarbon selected from the group consisting of n-butane, isobutane, pentane, neopentane, isopentane, and a combination of two or more of the foregoing hydrocarbons. In certain embodiments, for example, the one or plural cosolvents may comprise an ether selected from the group consisting of dimethyl ether, diethyl ether, and a combination of the two ethers. In certain embodiments, for example, the one or plural cosolvents may comprise a perfluorocarbon selected from the group consisting of perfluoropropane, perfluorobutane, perfluorocyclobutane, perfluoropentane, and a combination of two or more of the foregoing perfluorocarbons.

In certain embodiments, for example, the cosolvent may comprise ethanol at a concentration in the buffer in the range of 0.5-40 wt. %, for example at a concentration in the range of 0.5-10 wt. %, in the range of 10-20 wt. %, in the range of 20-30 wt. %, or the ethanol may be present in the buffer at a concentration in the range of 30-40 wt. %. In certain embodiments, for example, the buffer may comprise an ethanol concentration of at least 5 wt. %, at least 10 wt. %, at least 25 wt. %, or the buffer may comprise an ethanol concentration of at least 35 wt. %. In certain embodiments, for example, the buffer may comprise an ethanol concentration of less than 25 wt. %, for example an ethanol concentration of less than 10 wt. %, or the buffer may comprise an ethanol concentration of less than 5 wt. %.

In certain embodiments, for example, the buffer may comprise citric acid at a concentration in the range of 0.005-0.2 wt. %, for example at a concentration in the range of 0.01-0.15 wt. %, in the range of 0.015-0.1 wt. %, in the range of 0.025-0.075 wt. %, in the range of 0.04-0.06 wt. %, in the range of 0.06-0.08 wt. %, or the buffer may comprise citric acid at a concentration in the range of 0.055-0.065 wt. %. In certain embodiments, for example, the buffer may comprise citric acid at a concentration of less than 0.5 wt. %, for example a concentration of less than 0.25 wt. %, less than 0.1 wt. %, less than 0.075 wt. %, less than 0.06 wt. %, less than 0.03 wt. %, less than 0.02 wt. %, or the buffer may comprise citric acid at a concentration of less than 0.01 wt. %. In certain embodiments, for example, the buffer may comprise citric acid at a concentration of at least 0.01 wt. %, for example a concentration of at least 0.02 wt. %, at least 0.03 wt. %, at least 0.04 wt. %, or the buffer may comprise citric acid at a concentration of at least 0.06 wt. %.

In certain embodiments, for example, the buffer may comprise sodium chloride at a concentration in the range of 0.01-2 wt. %, for example at a concentration in the range of 0.25-1.5 wt. %, in the range of 0.5-1 wt. %, in the range of 0.6-0.7, in the range of 0.7-0.8 wt. %, or the buffer may comprise sodium chloride at a concentration in the range of 0.68-0.72 wt. %. In certain embodiments, for example, the buffer may comprise sodium chloride at a concentration of less than 1 wt. %, for example at a concentration of less than 0.72 wt. %.

In certain embodiments, for example, the buffer may comprise sodium citrate at a concentration in the range of 0.001-1 wt. %, for example at a concentration in the range of 0.01-0.75 wt. %, in the range of 0.1-0.6 wt. %, in the range of 0.25-0.5 wt. %, in the range of 0.3-0.4 wt. %, in the range of 0.4-0.5 wt. %, or the buffer may comprise sodium citrate at a concentration in the range of 0.35-0.45 wt. %. In certain embodiments, for example, the buffer may comprise sodium citrate at a concentration of less than 1 wt. %, for example a concentration of less than 0.75 wt. %, less than 0.6 wt. %, less than 0.5 wt. %, less than 0.45 wt. %, less than 0.4 wt. %, or the buffer may comprise sodium citrate at a concentration of less than 0.1 wt. %. In certain embodiments, for example, the buffer may comprise sodium citrate at a concentration of at least 0.1 wt. %, for example a concentration of at least 0.2 wt. %, at least 0.3 wt. %, at least 0.4 wt. %, or the buffer may comprise sodium citrate at a concentration of at least 0.5 wt. %.

In certain embodiments, for example, the buffer may be free, or substantially free (i.e., less than 0.008 wt. %), of preservative including, but not limited to, quaternary ammonium preservatives, such as a benzalkonium salt, (e.g., benzalkonium chloride). In certain embodiments, for example, the buffer may comprise less than 0.1 wt. % preservative (or quaternary ammonium preservative) (such as less than 0.05 wt. %, less than 0.02 wt. %, or less than 0.008 wt. % preservative).

In certain embodiments, for example, the buffer may be free, or substantially free (i.e., less than 0.008 wt. %), of complexing agent. In certain embodiments, for example, the buffer may comprise less than about 0.1 wt. % complexing agent (such as less than about 0.05 wt. %, less than about 0.02 wt. %, or less than about 0.008 wt. %), based on the weight of the buffer. In certain further embodiments, for example, the buffer may be free of ethylenediaminetetraacetic acid (EDTA). In certain embodiments, for example, the buffer may comprise EDTA at a concentration in the range of 0.0001-0.02 wt. % EDTA, for example at a concentration in the range of 0.0025-0.0175 wt. %, in the range of 0.0005-0.0015 wt. %, in the range of 0.0075-0.0125 wt. %, or the buffer may comprise EDTA at a concentration in the range of 0.009-0.012 wt. %. In certain embodiments, for example, the buffer may comprise EDTA at a concentration in the range of 0.0001-0.01 wt. % EDTA, for example at a concentration in the range of 0.00075-0.0075 wt. %, in the range of 0.001-0.0075 wt. %, in the range of 0.001-0.005 wt. %, or the buffer may comprise EDTA at a concentration in the range of 0.002-0.003 wt. %. In certain embodiments, for example, the buffer may comprise EDTA at a concentration of 0.0095 wt. %. In certain embodiments, for example, the buffer may comprise EDTA at a concentration of 0.003 wt. %. In certain embodiments, for example, the buffer may comprise EDTA at a concentration of less than 0.02 wt. %, for example at a concentration of less than 0.015 wt. %, less than 0.01 wt. %, a concentration of less than 0.005 wt. %, or the buffer may comprise EDTA at a concentration of less than 0.003 wt. %.

In certain embodiments, for example, the buffer may be free, or substantially free (i.e., less than 0.008 wt. %), of any component (for example any preservative (for example benzalkonium chloride), stabilizing agent, or complexing agent) which causes airway constriction in an ordinary subject when inhaled. In certain embodiments, for example, the buffer may be free, or substantially free (i.e., less than 0.008 wt. %) of any component (for example any preservative (for example benzalkonium chloride), stabilizing agent, or complexing agent) which reduces (or offsets) the effectiveness of tiotropium ordinary in an ordinary subject when inhaled.

In certain embodiments, for example, the tiotropium solution may have a pH in the range of 2-6, for example a pH in the range of 2-5, in the range of 2-4.5, in the range of 2.5-3.5, in the range of 2.7-3.2, or the tiotropium solution may have a pH in the range of 2.8-3. In certain embodiments, for example, the pH of the tiotropium solution may be adjusted by adding a quantity of one or more pharmaceutically acceptable acids. In certain embodiments, for example, the one or more pharmaceutically acceptable acids may be an inorganic acid, such as hydrochloric acid, hydrobromic acid, nitric acid, sulfuric acid, and phosphoric acid, or a combination of two or more of the foregoing acids. In certain embodiments, for example, the one or more pharmaceutically acceptable acids may comprise one or more organic acids, such as ascorbic acid, citric acid, malic acid, maleic acid, tartaric acid, succinic acid, fumaric acid, acetic acid, formic acid, propionic acid, or a combination of two or more of the foregoing acids. In certain embodiments, for example, the pH of the tiotropium solution may be adjusted by adding a quantity of 1 N hydrochloric acid or 1 N sulfuric acid. In certain embodiments, for example, the pH of the tiotropium solution may be adjusted by adding a quantity of one or more organic acids selected from the group consisting of ascorbic acid, fumaric acid, citric acid, and combinations of two or more of the foregoing acids. In certain embodiments, for example, mixtures of two or more of the above-mentioned acids may be used.

In certain embodiments, for example, the tiotropium solution may be iso-osmolal with respect to fluids in the lungs. In certain embodiments, for example, the tiotropium solution may be iso-osmolal with respect to fluids in the eye. In certain embodiments, for example, the tiotropium solution may be iso-osmolal with respect to fluids in the nose. In certain embodiments, for example, the tiotropium solution may have an osmolality in the range of 200-500 mOsm/kg (for example as determined by a freezing point depression measurement), for example the tiotropium solution may have an osmolality in the range of 175-330 mOsm/kg, in the range of 275-325 mOsm/kg, or the tiotropium solution may have an osmolality in the range of 280-320 mOsm/kg. In certain embodiments, for example, the osmolality and/or tonicity of the tiotropium solution may be adjusted by adding a quantity of ammonium carbonate, ammonium chloride, ammonium lactate, ammonium nitrate, ammonium phosphate, ammonium sulfate, ascorbic acid, bismuth sodium tartrate, boric acid, calcium chloride, calcium disodium edetate, calcium gluconate, calcium lactate, citric acid, dextrose, diethanolamine, dimethyl sulfoxide, edetate disodium, edetate trisodium monohydrate, fluorescein sodium, fructose, galactose, glycerin, lactic acid, lactose, magnesium chloride, magnesium sulfate, mannitol, PEG (for example PEG 300), potassium acetate, potassium chlorate, potassium chloride, potassium iodide, potassium chloride, potassium nitrate, potassium phosphate, potassium sulfate, propylene glycol, silver nitrate, sodium acetate, sodium bicarbonate, sodium biphosphate, sodium bisulfite, sodium borate, sodium bromide, sodium cacodylate, sodium carbonate, sodium chloride, sodium citrate, sodium iodide, sodium lactate, sodium metabisulfite, sodium nitrate, sodium nitrite, sodium phosphate, sodium propionate, sodium succinate, sodium sulfate, sodium sulfite, sodium tartrate, sodium thiosulfate, sorbitol, sucrose, tartaric acid, triethanolamine, urea, urethan, uridine, zinc sulfate, one or more monosaccharides, or a combination of two or more of the foregoing components. In certain embodiments, for example, the tiotropium solution may be isotonic with respect to fluids in the lungs. In certain embodiments, for example, the tiotropium solution may be isotonic with respect to fluids in the eye. In certain embodiments, for example, the tiotropium solution may be isotonic with respect to fluids in the nose.

In certain embodiments, for example, the buffer may comprise one or more of acetate, barbital, borate, Britton-Robinson, cacodylate, citrate, collidine, formate, maleate, McMaine, phosphate, PrideauxWard, succinate, citrate-phosphate-borate (Teorell-Stanhagen), veronal acetate, MES, BIS-TRIS, ADA, ACES, PIPES, MOPSO, BIS-TRIS PROPANE, BES, MOPS, TES, HEPES, DIPSO, MOBS, TAPSO, TRIZMA, HEPPSO, POPSO, TEA, EPPS, TRICINE, GLY-GLY, BICINE, HEPBS, TAPS, orAMPD buffer. In certain embodiments, for example, the buffer may consist of acetate, barbital, borate, Britton-Robinson, cacodylate, citrate, collidine, formate, maleate, McMaine, phosphate, PrideauxWard, succinate, citrate-phosphate-borate (Teorell-Stanhagen), veronal acetate, MES, BIS-TRIS, ADA, ACES, PIPES, MOPSO, BIS-TRIS PROPANE, BES, MOPS, TES, HEPES, DIPSO, MOBS, TAPSO, TRIZMA, HEPPSO, POPSO, TEA, EPPS, TRICINE, GLY-GLY, BICINE, HEPBS, TAPS, or AMPD buffer.

In certain embodiments, for example, the tiotropium solution may comprise one or more surfactants. In certain embodiments, for example, the one or more surfactants may comprise C5-20 fatty alcohols, C5-20 fatty acids, C5-20 fatty acid esters, lecithin, glycerides, propylene glycol, esters, polyoxyethylenes, polysorbates, sorbitan esters and/or carbohydrates, or a combination of two or more of the foregoing surfactants.

In certain embodiments, for example, the tiotropium solution may comprise one or more antioxidants. In certain embodiments, for example, the one or more antioxidants may comprise ascorbic acid, vitamin A, vitamin E, tocopherols, vitamins or pro-vitamins occurring in the human body, or a combination of two or more of the foregoing antioxidants.

In certain embodiments, for example, the tiotropium solution may comprise one or more ingredients (for example one or more, such as all, inactive ingredients present in the tiotropium solution) at a concentration falling within limits defined by the United States Food and Drug Administration Inactive Ingredients Database and/or Inactive Ingredient Guide.

In certain embodiments, for example, the tiotropium solution may be free, or substantially free (i.e., less than 0.008 wt. %), of solids. In certain embodiments, for example, tiotropium solution may comprise less than 0.1 wt. % solids (such as less than 0.05 wt. %, less than 0.02 wt. %, or less than 0.008 wt. % solids), based on the total weight of the tiotropium solution. In certain embodiments, for example, the solids may comprise a precipitate. In certain embodiments, for example, the solids may comprise a flocculate. In certain embodiments, for example, the solids may comprise a residue. In certain embodiments, for example, the solids may comprise an impurity. In certain embodiments, for example, the solids may form in the tiotropium solution after a period of time (for example after 3 days, 7 days, 2 weeks, 3 weeks, 1 month, 3 months, 6 months, 12 months, 18 months, 24 months, or 36 months). In certain embodiments, for example, the solids may form after heating the tiotropium solution (for example heating from 25° C. to 40° C., from 25° C. to 60° C., or from 25° C. to a temperature greater than 40° C.). In certain embodiments, for example, the solids may form after exposing the tiotropium solution to oxygen.

In certain embodiments, for example, the tiotropium solution (or a second tiotropium solution) may be a drug product. In certain further embodiments, for example, the drug product may be a sterile nebulizable pharmaceutical solution for inhalation via nebulization. In certain embodiments, for example, the drug product may be a sterile ophthalmic solution. In certain embodiments, for example, the drug product may be a sterile nasal spray. In certain embodiments, for example, the drug product may be a sterile topical solution. In certain embodiments, for example, the drug product may be a sterile solution suitable for intravenous injection or injection into tissue. In certain embodiments, for example, the tiotropium solution (or the second tiotropium solution) may be dried (for example spray dried or freeze-dried) to form a sterile powdered drug product (for example a powdered drug product suitable for delivery by nasal or pulmonary inhalation).

In certain embodiments, for example, the tiotropium solution (for example a master batch or a drug product) may have a long shelf life (for example an 18 month shelf life when stored in a plastic semi-permeable container in dark conditions at a temperature of 25° C. and 40-75% relative humidity (for example 40% relative humidity or 60% relative humidity)). In certain embodiments, for example, the tiotropium solution may be stable during long-term storage. In certain embodiments, for example, the tiotropium solution may contain greater than 80% of an initial quantity of tiotropium following storage for a period of time, relative to a quantity of tiotropium initially present in the tiotropium solution, for example greater than 85%, greater than 90%, greater than 95%, or the tiotropium solution may contain or greater than 98% of an initial quantity of tiotropium following storage for a period of time, relative to a quantity of tiotropium initially present in the tiotropium solution. In certain further embodiments, for example, the storage may be at a temperature of 25° C. and the period of time may be 3 months, 6 months, 1 year, 2 years, or the period of time may be 3 years. In certain embodiments, for example, the storage may be at a temperature of 30° C. and the period of time may be 1 week, 2 weeks, 1 month, 2 months, 3 months, 4 months, 5 months, 6 months, 7 months, 8 months, 9 months, 10 months, 11 months, 12 months, 18 months, or the period of time may be 24 months. In certain embodiments, for example, the storage may be at a temperature of 40° C. and the period of time may be 1 week, 2 weeks, 1 month, 2 months, 3 months, 4 months, 5 months, 6 months, 7 months, 8 months, 9 months, 10 months, 11 months, 12 months, 18 months, or the period of time may be 24 months. In certain embodiments, for example, the storage may be at a temperature of 60° C. and the period of time may be 1 week, 2 weeks, 1 month, 2 months, or the period of time may be 3 months. In certain embodiments, for example, the storage may be at a specified relative humidity. In certain embodiments, for example, the specified relative humidity may be in the range of 10-90%, for example in the range of 20-30%, in the range of 30-50%, or the specified relative humidity may be in the range of 50-80%. In certain embodiments, for example, the specified relative humidity may be 40%, 60%, 65%, or 75%. In certain embodiments, for example, the storage temperature may be 25° C. and the specified relative humidity may be 60%. In certain embodiments, for example, the storage temperature may be 30° C. and the specified relative humidity may be 65%. In certain embodiments, for example, the storage temperature may be 40° C. and the specified relative humidity may be 75%. In certain embodiments, for example, the storage may be under low light or dark conditions (for example the container may be placed in an opaque wrapper such as a foil wrapper).

In certain embodiments, for example, a portion of a sample of the tiotropium solution may be passed through an 0.2 micron polyvinylidene fluoride filter. In certain embodiments, for example, the tiotropium content of the filtered portion may be reduced by less than 10% (as determined, for example, using high-performance liquid chromatography) compared to an unfiltered portion of the sample, for example reduced by less than less than 8%, less than 4%, less than 2%, less than 1%, or the tiotropium content of the filtered portion may be reduced by less than 0.5% when the tiotropium solution is passed through an 0.2 micron polyvinylidene fluoride filter. In certain embodiments, for example, the example, the portion of the sample passed through the filter may have been exposed to dark (no light) conditions at temperature of 25° C. and 40% or 60% relative humidity for 6 months, 1 year, or 2 years. In certain further embodiments, for example, the tiotropium solution may be stored (prior to filtration) in a glass or blow-fill-seal container having an impermeable or semipermeable lid.

In certain embodiments, for example, a portion of a sample of the tiotropium solution may be passed through an 0.2 micron polyvinylidene fluoride filter. In certain embodiments, for example, the tiotropium content of the filtered portion may be reduced by less than 10% (as determined, for example, using high-performance liquid chromatography) compared to an unfiltered portion of the sample, for example reduced by less than less than 8%, less than 4%, less than 2%, less than 1%, or the tiotropium content of the filtered portion may be reduced by less than 0.5% when the tiotropium solution is passed through an 0.2 micron polyvinylidene fluoride filter.

In certain embodiments, for example, the storage may be in a glass container (for example a sterilized glass container). In certain embodiments, for example, the storage may be in a plastic container (for example a sterilized plastic container). In certain embodiments, for example, the plastic container may be a low density polyethylene container. In certain embodiments, for example, the plastic container may be a sterile, blow-fill-seal polyethylene container that is semipermeable to air and impermeable to microorganisms. In certain embodiments, for example, the storage may be in a cyclic olefin polymer container. In certain embodiments, for example, the storage may be in a cyclic olefin copolymer container. In certain embodiments, for example, the storage may be in a unit dose container. In certain embodiments, for example, the storage may be in a unit dose blow-fill-seal container. In certain embodiments, for example, the unit dose blow-fill-seal container may be contained in a foil pouch (for example a sealed opaque foil pouch). In certain embodiments, for example, the sterile, stable, tiotropium solution may be sterile and remain sterile during the storage. In certain further embodiments, for example, the sterile, stable, tiotropium solution may be preservative-free or substantially preservative-free. In certain embodiments, for example, the sterile, stable, tiotropium solution may be benzalkonium chloride-free or substantially benzalkonium chloride-free.

In certain embodiments, for example, a unit dose of the tiotropium solution may retain greater than 85 wt. % of an initial quantity of tiotropium (for example greater than 95 wt. % or greater than 98 wt. %) and remain sterile when stored for 24 months in a unit dose, semi-permeable blow-fill-seal container under low light or no light (dark conditions) at 25° C. and 40% or 60% relative humidity. In certain embodiments, for example, a unit dose of the tiotropium solution may retain greater than 85 wt. % of an initial quantity of tiotropium (for example greater than 95 wt. % or greater than 98 wt. %) and remain sterile when stored for 1 month, 2 months, 3 months, or 6 months in a unit dose, semi-permeable blow-fill-seal container under low light or no light (dark) conditions at 40° C. and 75% relative humidity.

In certain embodiments, for example, the tiotropium solution may be nebulized to form droplets having an average size in the range of 0.1-10 microns (for example as determined by a next generator impactor), for example droplets having an average size in the range of 1-6 microns, in the range of 1-5 microns, in the range of 2-6 microns, or the solution may be nebulized to form droplets having an average size in the range of 0.5-5 microns when passed through a Pari LC Jet Plus Nebulizer connected to a Pari Master or Pari VIOS compressor. In certain further embodiments, for example, the nebulizer may be a vibrating mesh nebulizer. In certain further embodiments, for example, the nebulizer may be a battery-powered, hand-held vibrating mesh nebulizer.

Certain embodiments may provide, for example, a unit dose container that contains a unit dose of the tiotropium solution (for example a tiotropium drug product). In certain further embodiments, for example, the unit dose may have a volume in the range of 0.1-6 mL, for example a volume in the range of 0.5-3 mL, such as a unit dose volume of 0.5 mL, 1 mL, or 2 mL. In certain embodiments, for example, the unit dose container may contain a volume of the tiotropium solution comprising a therapeutic quantity of tiotropium. In certain further embodiments, for example, the unit dose container may contain in the range of 0.25 mcg to no more than 10 mcg of tiotropium, for example in the range of 0.25 mcg to no more than 0.5 mcg of tiotropium, in the range of 0.5 mcg to no more than 0.75 mcg, in the range of 0.75 mcg to no more than 1 mcg, in the range of 1 mcg to no more than 1.25 mcg, in the range of 1.25 mcg to no more than 2 mcg, in the range of 2 mcg to no more than 2.25 mcg, in the range of 2.25 mcg to no more than 2.5 mcg, in the range of 2.5 mcg to no more than 2.625 mcg, in the range of 2.625 mcg to no more than 3 mcg, in the range of 3 mcg to no more than 5 mcg, in the range of 5 mcg to no more than 8 mcg, or the unit dose container may contain in the range of 8 mcg to no more than 10 mcg of tiotropium. In certain embodiments, for example, any of the foregoing therapeutic quantities of tiotropium may be present in a 0.5 mL unit dose volume of the tiotropium solution. In certain embodiments, for example, any of the foregoing therapeutic quantities of tiotropium may be present in a 1 mL unit dose volume of the tiotropium solution. In certain embodiments, for example, any of the foregoing therapeutic quantities of tiotropium may be present in a 2 mL unit dose volume of the tiotropium solution. In certain embodiments, for example, the unit dose may be defined by 250 mcg of tiotropium in 2 mL of the tiotropium solution. In certain embodiments, for example, the unit dose may be defined by 500 mcg of tiotropium in 2 mL of the tiotropium solution. In certain embodiments, for example, the unit dose may be defined by 1000 mcg of tiotropium, in 2 mL of the tiotropium solution.

Certain embodiments may provide, for example, a unit dose container that contains a unit dose of the tiotropium solution (for example a tiotropium drug product such as a sterile, preservative-free, complexing agent-free unit dose of the tiotropium solution). In certain further embodiments, for example, the unit dose may have a volume in the range of 0.1-6 mL, for example a volume in the range of 0.5-3 mL, such as a unit dose volume of 0.5 mL, 1 mL, or 2 mL. In certain embodiments, for example, the unit dose container may contain a volume of the tiotropium solution comprising a therapeutic quantity of tiotropium. In certain further embodiments, for example, the unit dose container may contain in the range of 0.25 mcg to no more than 20 mcg of tiotropium, for example in the range of 0.25 mcg to no more than 0.5 mcg of tiotropium, in the range of 0.5 mcg to no more than 0.75 mcg, in the range of 0.75 mcg to no more than 1 mcg, in the range of 1 mcg to no more than 1.25 mcg, in the range of 1.25 mcg to no more than 2 mcg, in the range of 2 mcg to no more than 2.25 mcg, in the range of 2.25 mcg to no more than 2.5 mcg, in the range of 2.5 mcg to no more than 2.625 mcg, in the range of 2.625 mcg to no more than 3 mcg, in the range of 3 mcg to no more than 5 mcg, in the range of 5 mcg to no more than 8 mcg, in the range of 8 mcg to no more than 10 mcg, in the range of 10 mcg to more than 11 mcg, in the range of 11 mcg to no more than 12 mcg, in the range of 12 mcg to no more than 13 mcg, in the range of 13 mcg to no more than 14 mcg, in the range of 14 mcg to no more than 15 mcg, in the range of 15 mcg to no more than 16 mcg, in the range of 16 mcg to no more than 17 mcg, in the range of 17 mcg to no more than 18 mcg, in the range of 18 mcg to no more than 19 mcg, or the unit dose container may contain in the range of 19 mcg to no more than 20 mcg of tiotropium. In certain further embodiments, for example, the unit dose container may contain 5 mcg of tiotropium, for example 5.1 mcg, 5.2 mcg, 5.3 mcg, 5.4 mcg, 5.5 mcg, 5.6 mcg, 5.7 mcg, 5.8 mcg, 5.9 mcg, 6 mcg, 6.1 mcg, 6.2 mcg, 6.3 mcg, 6.4 mcg, 6.5 mcg, 6.6 mcg, 6.7 mcg, 6.8 mcg, 6.9 mcg, 7 mcg, 7.1 mcg, 7.2 mcg, 7.3 mcg, 7.4 mcg, 7.5 mcg, 7.6 mcg, 7.7 mcg, 7.8 mcg, 7.9 mcg, 8 mcg, 8.1 mcg, 8.2 mcg, 8.3 mcg, 8.4 mcg, 8.5 mcg, 8.6 mcg, 8.7 mcg, 8.8 mcg, 8.9 mcg, 9 mcg, 9.1 mcg, 9.2 mcg, 9.3 mcg, 9.4 mcg, 9.5 mcg, 9.6 mcg, 9.7 mcg, 9.8 mcg, 9.9 mcg, 10 mcg, 10.1 mcg, 10.2 mcg, 10.3 mcg, 10.4 mcg, 10.5 mcg, 10.6 mcg, 10.7 mcg, 10.8 mcg, 10.9 mcg, 11 mcg, 11.1 mcg, 11.2 mcg, 11.3 mcg, 11.4 mcg, 11.5 mcg, 11.6 mcg, 11.7 mcg, 11.8 mcg, 11.9 mcg, 12 mcg, 12.1 mcg, 12.2 mcg, 12.3 mcg, 12.4 mcg, 12.5 mcg, 12.6 mcg, 12.7 mcg, 12.8 mcg, 12.9 mcg, 13 mcg, 13.1 mcg, 13.2 mcg, 13.3 mcg, 13.4 mcg, 13.5 mcg, 13.6 mcg, 13.7 mcg, 13.8 mcg, 13.9 mcg, 14 mcg, 14.1 mcg, 14.2 mcg, 14.3 mcg, 14.4 mcg, 14.5 mcg, 14.6 mcg, 14.7 mcg, 14.8 mcg, 14.9 mcg, 15 mcg, 15.1 mcg, 15.2 mcg, 15.3 mcg, 15.4 mcg, 15.5 mcg, 15.6 mcg, 15.7 mcg, 15.8 mcg, 15.9 mcg, 16 mcg, 16.1 mcg, 16.2 mcg, 16.3 mcg, 16.4 mcg, 16.5 mcg, 16.6 mcg, 16.7 mcg, 16.8 mcg, 16.9 mcg, 17 mcg, 17.1 mcg, 17.2 mcg, 17.3 mcg, 17.4 mcg, 17.5 mcg, 17.6 mcg, 17.7 mcg, 17.8 mcg, 17.9 mcg, 18 mcg, 18.1 mcg, 18.2 mcg, 18.3 mcg, 18.4 mcg, 18.5 mcg, 18.6 mcg, 18.7 mcg, 18.8 mcg, 18.9 mcg, 19 mcg, 19.1 mcg, 19.2 mcg, 19.3 mcg, 19.4 mcg, 19.5 mcg, 19.6 mcg, 19.7 mcg, 19.8 mcg, 19.9 mcg, or 20 mcg of tiotropium. In certain embodiments, for example, any of the foregoing quantities (for example the quantities may be therapeutic quantities) of tiotropium may be present in a 0.5 mL unit dose volume of the tiotropium solution. In certain embodiments, for example, any of the foregoing quantities (for example the quantities may be therapeutic quantities) of tiotropium may be present in a 1 mL unit dose volume of the tiotropium solution. In certain embodiments, for example, any of the foregoing quantities (for example the quantities may be therapeutic quantities) of tiotropium may be present in a 2 mL unit dose volume of the tiotropium solution. In certain embodiments, for example, the unit dose may be defined by 250 mcg of tiotropium in 2 mL of the tiotropium solution. In certain embodiments, for example, the unit dose may be defined by 500 mcg of tiotropium in 2 mL of the tiotropium solution. In certain embodiments, for example, the unit dose may be defined by 1000 mcg of tiotropium, in 2 mL of the tiotropium solution.

In certain embodiments, for example, the unit dose container may be prepackaged. In certain embodiments, for example, the unit dose container may be sterile. In certain embodiments, for example, the unit dose container may contain a ready-to-use quantity of the tiotropium solution. In certain further embodiments, for example, the ready-to-use quantity of the tiotropium solution may not require any mixing or dilution prior to administration. In certain embodiments, for example, the unit dose container may contain a sterile, therapeutically effective quantity of tiotropium, for the treatment, prevention, or amelioration of one or more symptoms of a disease or condition that causes a constriction or narrowing of the bronchi (for example a bronchoconstrictive disorder). In certain embodiments, for example, the disease or condition may be asthma, pediatric asthma, bronchial asthma, allergic asthma, intrinsic asthma, chronic obstructive pulmonary disease (COPD), chronic bronchitis, emphysema, or a combination of two or more of the foregoing diseases or conditions. In certain embodiments, for example, the unit dose container may contain a sterile, therapeutically effective quantity of tiotropium inhalation solution (for example a unit dose containing 5 mcg of tiotropium in solution) for long-term, once-daily maintenance treatment of bronchospasm associated with COPD and/or reducing COPD exacerbations. In certain embodiments, for example, the unit dose container may contain a sterile, therapeutically effective quantity of tiotropium inhalation solution (for example a unit dose containing 2.5 mcg of tiotropium in solution) for long-term, once-daily maintenance treatment of asthma (for example asthma in patients of 6 years of age or older).

In certain embodiments, for example, the unit dose container may be formed aseptically using a blow-fill-seal process, wherein the container is formed, filled with a sterile volume of the tiotropium solution, and sealed in a continuous process without human intervention, in a sterile enclosed area inside a machine. In certain embodiments, for example, the blow-fill-seal process may comprise a) vertically heat extruding a pharmaceutical-grade plastic through a circular throat to form a parison (i.e., a tube such as a hanging tube); b) enclosing the extruded tube within a two-part mold; c) cutting the tube above the mold; transferring the mold to a sterile filling space, wherein one or more mandrels (i.e., filling needles) are lowered and used to inflate the plastic to form the container within the mold; d) filling the container, using the one or more filling needles, with the tiotropium solution; e) retracting the one or more filling needles; and f) forming a top in a secondary top mold to seal the container. In certain embodiments, for example, the process may comprise sterilization (for example sterile filtration) of the tiotropium solution prior to the filling. In certain embodiments, for example, the process may be exclusive of sterilization (for example thermal sterilization) following the filling. In certain embodiments, for example, the pharmaceutical-grade plastic may be polyethylene. In certain embodiments, for example, the pharmaceutical-grade plastic may be polypropylene.

Certain embodiments may provide, for example, a method to treat, prevent, or ameliorate one or more symptoms of a disease or condition that causes a constriction or narrowing of the bronchi (for example a bronchoconstrictive disorder). In certain embodiments, for example, the disease or condition may be asthma, pediatric asthma, bronchial asthma, allergic asthma, intrinsic asthma, chronic obstructive pulmonary disease (COPD), chronic bronchitis, and emphysema. In certain embodiments, for example, the method may comprise nebulization of one of the tiotropium solutions disclosed herein. In certain embodiments, for example, the method may comprise inhalation of one of the tiotropium solutions disclosed herein by a mammal, for example a human subject. In certain embodiments, for example, the method may comprise daily (for example once daily, twice daily, three times daily, or four times daily) nebulization of one of the tiotropium solutions disclosed herein by a mammal (for example a human subject in need of treatment). In certain further embodiments, for example, the method may comprise nebulization of at least a portion of a volume (for example a portion of 0.5 mL, 1 mL, 2 mL, or 4 mL) of the tiotropium solution using a nebulizer. In certain embodiments, for example, the nebulizer may be a jet nebulizer (for example an air-driven jet nebulizer or a jet nebulizer connected to an air compressor such as Pari LC Jet Plus Nebulizer connected to a Pari Master of Pari VIOS compressor). In certain embodiments, for example, the nebulizer may be an ultrasonic nebulizer. In certain embodiments, for example, the nebulizer may be a vibrating mesh nebulizer. In certain embodiments, for example, the nebulizer may be a breath-actuated nebulizer.

Certain embodiments, for example, may provide a method of treatment for subjects who find it difficult to use an inhaler. In certain embodiments, for example, the method may comprise administering one of the tiotropium solutions disclosed herein with one of the nebulizers disclosed herein (for example a jet nebulizer or a vibrating mesh nebulizer). In certain further embodiments, for example, the subject may be a pediatric patient. In certain further embodiments, for example, the subject may be a geriatric patient. In certain further embodiments, for example, the subject may be a patient with poor hand-inhalation coordination.

Certain embodiments may provide, for example, a kit to treat, prevent, or ameliorate one or more symptoms of a disease or condition that causes a constriction or narrowing of the bronchi (for example a bronchoconstrictive disorder). In certain embodiments, for example, the kit may comprise at least one (for example five) sterile unit dose container (for example a blow-fill-seal plastic container with a twist-off cap) containing a unit dose of the tiotropium solution, the unit dose having a therapeutically effective quantity tiotropium. In certain embodiments, for example, the kit may further comprise a foil pouch (for example an opaque aluminum foil pouch) that contains the at least one sterile unit dose container. In certain embodiments, for example, the kits may contain instructions for use. In certain embodiments, for example, the kit may contain a medicine cup component of a hand-held vibrating mesh nebulizer. In certain embodiments, for example, the kit may contain a hand-held, battery-powered nebulizer.

### EXAMPLES

In the following Examples:

“q.s.” refers to a quantity of buffer sufficient to bring the listed components to the concentrations indicated;

“n.a.” means “not applicable”;

and “-” or “--” indicates no data presented.

### Preparation of Tiotropium Nebulization Solutions

A stock buffer solution was prepared by dissolving sodium chloride, citric acid monohydrate, and sodium citrate dihydrate in purified water. To prepare each tiotropium nebulization solution, a quantity of the stock buffer solution was added to a volumetric flask and mixed with a quantity of amorphous, anhydrous tiotropium bromide under stirring, followed by diluting with purified water to the desired tiotropium concentration. The prepared solutions are presented in Table 1.

TABLE 1
Tiotropium Nebulization Solutions

| Component | A | B | C | D | E | F |
|---|---|---|---|---|---|---|
| Tiotropium (mcg/mL) | 4.4 | 5.0 | 5.6 | 17.6 | 20.0 | 22.4 |
| Sodium Chloride | 0.8 | 0.8 | 0.8 | 0.8 | 0.8 | 0.8 |
| (wt. %) | | | | | | |
| Sodium Citrate | 0.1 | 0.1 | 0.1 | 0.1 | 0.1 | 0.1 |
| Dihydrate (wt. %) | | | | | | |
| Citric Acid | 0.4 | 0.4 | 0.4 | 0.4 | 0.4 | 0.4 |
| Monohydrate (wt. % | | | | | | |
| Citric acid anhydrous) | | | | | | |
| Water (wt. %) | q.s. | q.s. | q.s. | q.s. | q.s. | q.s. |

### Delivered Dose Testing

In a series of examples, six different tiotropium bromide solutions were nebulized by two types of nebulizers (a Pari LC Sprint Nebulizer part no. 023F35 connected to a Pari VIOS compressor part no. 310B0003 produced by Pari Respiratory Equipment, Inc. and a Pocket Neb® Model MVD-70 vibrating mesh nebulizer produced by MicroVapor Devices, LLC) and passed through a breathing simulator. In each example, a deposited quantity of aerosol was collected from the breathing simulator and tiotropium bromide content determined via high pressure liquid chromatography. Breathing simulator parameters are given in Table 2 and results are presented in Table 3. In comparative examples, 1.25 μg and 2.5 μg Spiriva® Respimat were delivered via a Soft Mist™ inhaler (two actuations per replicate) into a Copley Metered Dose Sampling Tube (DUSA Tube) at a constant flow of 28.3 L/min and results are presented in Table 3.

TABLE 2
Breathing Simulator Parameters

| Parameter | Setting |
|---|---|
| Volume Inhaled per Inhalation | 500 mL |
| Breathing Pattern | Sinusoidal |
| Breaths Per Minute | 15 |
| Inspiratory to Expiratory (I:E) Ratio | 1:1 |

TABLE 3
Tiotropium Dosage

| | | | Fill | | Tiotropium |
|---|---|---|---|---|---|
| | | | Volume | Time | Delivered |
| Example¹ | Device | Composition | (mL) | (min)^{3, 4} | (mcg) |
| Example 1 | Pocket | A | 2 | 3:43 | 2.3 |
| | Neb ® | | | | |
| Example 2 | LC ® | A | 2 | 5:00 | 2.2 |
| Example 3 | Pocket | B | 2 | 4:24 | 2.8 |
| | Neb ® | | | | |
| Example 4 | LC ® | B | 2 | 5:00 | 2.5 |
| Example 5 | Pocket | C | 2 | 4:23 | 3.1 |
| | Neb ® | | | | |
| Example 6 | LC ® | C | 2 | 5:00 | 2.7 |
| Example 7 | Pocket | D | 0.5 | 1:01 | 2.2 |
| | Neb ® | | | | |
| Example 8 | Pocket | E | 0.5 | 1:04 | 2.6 |
| | Neb ® | | | | |
| Example 9 | Pocket | F | 0.5 | 0:55 | 3.0 |
| | Neb ® | | | | |
| Comparative | Soft | 1.25 μg | N/A | N/A | 2.4² |
| Example A | Mist ™ | Spiriva ® | | | |
| | Inhaler | Respimat | | | |
| Comparative | Soft | 2.5 μg | N/A | N/A | 4.4² |
| Example B | Mist ™ | Spiriva ® | | | |
| | Inhaler | Respimat | | | |

¹Each example and comparative example was replicated ten times. All results are averages.
²Based on two actuations of the Soft Mist ™ Inhaler.
³Pocket Neb ® nebulizer shut off when low volume detected in reservoir.
⁴LC ® nebulizer shut off at 5 minutes leaving a residual volume of approximately 0.9 mL.

### Prophetic Examples

### Prophetic Examples 1-8

In the following prophetic examples, delivered doses of tiotropium compositions via nebulization of a volume of tiotropium solution would be determined for a Pari LC Sprint Nebulizer part no. 023F35 connected to a Pari VIOS compressor part no. 310B0003 (produced by Pari Respiratory Equipment, Inc.) (the “Pari System”) and a Pocket Neb® Model MVD-70 vibrating mesh nebulizer (produced by MicroVapor Devices, LLC) (the “Pocket system”). Compositions that would be tested are presented in Table 4 and results that would be obtained are presented in Table 5.

TABLE 4
Tiotropium Compositions

Composition

| | A | B | C | D |
|---|---|---|---|---|
| Total Volume (mL) | 2 | 2 | 2 | 2 |
| Tiotropium Bromide | 5 | 8.8 | 11.2 | 17.6 |
| (amorphous, | | | | |
| anhydrous) (mcg | | | | |
| Tiotropium) | | | | |
| Sodium Chloride (wt. %) | 0.8 | 0.8 | 0.8 | 0.8 |
| Sodium Citrate | 0.1 | 0.1 | 0.1 | 0.1 |
| Dihydrate (wt. %) | | | | |
| Citric Acid | 0.4 | 0.4 | 0.4 | 0.4 |
| Monohydrate (wt. % | | | | |
| Citric acid anhydrous) | | | | |
| Water (wt. %) | q.s. | q.s. | q.s. | q.s. |

TABLE 5
Tiotropium Dosage

| | | | | Tiotropium |
|---|---|---|---|---|
| Prophetic | | | Time | Delivered |
| Example | Nebulizer | Composition | (min) | (mcg) |
| 1 | Pocket system | A | 4 | 0.9 |
| 2 | Pari system | A | 5 | 1.1 |
| 3 | Pocket system | B | 4 | 2.3 |
| 4 | Pari system | B | 5 | 2.2 |
| 5 | Pocket system | C | 4 | 3.1 |
| 6 | Pari system | C | 5 | 2.7 |
| 7 | Pocket system | D | 4 | 5.2 |
| 8 | Pari system | D | 5 | 4.5 |

Unless otherwise specified, a specified quantity, weight percentage, or concentration of tiotropium in a solution means the quantity, weight percentage, or concentration based on the molecular weight of tiotropium (392.508 g/mol) (not the molecular weight of a salt and/or a hydrate of tiotropium if such other salt and/or hydrate used).

As used herein, citric acid means citric acid or a hydrate thereof. Unless otherwise specified, a specified quantity, weight percentage, or concentration of citric acid or a hydrate thereof in a solution means the quantity, weight percentage, or concentration based on the molecular weight of anhydrous citric acid (not the molecular weight of a hydrate of citric acid if such a hydrate is used). A specified quantity, weight percentage, or concentration referring specifically to a hydrate of citric acid means the quantity, weight percentage, or concentration based on the molecular weight of the specified hydrate.

As used herein, sodium citrate means sodium citrate or a hydrate thereof. Unless otherwise specified, a specified quantity, weight percentage, or concentration of sodium citrate or a hydrate thereof in a solution means the quantity, weight percentage, or concentration based on the molecular weight of sodium citrate dihydrate (not the molecular weight of anhydrous sodium citrate if such is used). A specified quantity, weight percentage, or concentration referring specifically to a hydrate of sodium citrate means the quantity, weight percentage, or concentration based on the molecular weight of the specified hydrate.

As used herein, ethylenediaminetetraacetic acid (EDTA) refers to ethylenediaminetetraacetic acid or a salt thereof (for example disodium edetate). Unless otherwise specified, a specified quantity, weight percentage, or concentration of EDTA means the quantity, weight percentage, or concentration based on the molecular weight of EDTA (not the molecular weight of the salt if such a salt is employed). A specified quantity, weight percentage, or concentration referring specifically to a salt of EDTA means the quantity, weight percentage, or concentration based on the molecular weight of the specified salt.

Unless specified otherwise, a weight percentage of a component of a composition means the weight percentage, on an as-added basis, relative to the total weight of the composition. For example, “a composition comprising 1 wt. % tiotropium” means 1 wt. % of tiotropium was added (regardless of whether or not the tiotropium undergoes a chemical transformation once present in the composition) relative to the total weight of the composition.

While preferred embodiments of the present invention have been shown and described herein, it will be obvious to those skilled in the art that such embodiments are provided by way of example only. It is intended that the following claims define the scope of the invention and that methods and structures within the scope of these claims and their equivalents be covered thereby.

What is claimed is:

1. An benzalkonium chloride-free, EDTA-free pharmaceutical composition comprising:
i) 0.0007-0.00088 wt. % tiotropium;
ii) 0.08-0.2 wt. % sodium citrate;
iii) 0.2-0.6 wt. % citric acid;
iv) 0.6-0.9 wt. % sodium chloride; and
v) 97-99.2 wt. % water.

2. The composition of claim 1, wherein the composition is complexing agent-free.

3. The composition of claim 2, wherein the composition is preservative-free.

4. The composition of claim 3, wherein a 2 mL dose of the preservative-free, complexing agent-free pharmaceutical composition containing 14-17.6 mcg of tiotropium dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid.

5. The composition of claim 4, wherein the nebulized dose of the pharmaceutical composition is bioequivalent when compared to:
(a) an AUC of tiotropium of the inhaler-dispensed dose of the tiotropium solution; and
(b) a Cₘₐₓ of tiotropium of the inhaler-dispensed dose of the tiotropium solution.

6. The composition of claim 4, wherein the nebulized dose of the pharmaceutical composition provides:
(a) an AUC of tiotropium in the range of 80-125% of an AUC of tiotropium of the inhaler-dispensed dose of the tiotropium solution; and
(b) a Cₘₐₓ of tiotropium in the range of 80-125% of a Cₘₐₓ of tiotropium of the inhaler-dispensed dose of the tiotropium solution.

7. The composition of claim 4, wherein the nebulized dose of the pharmaceutical composition provides: (a) an AUC of tiotropium of 25.7-40.2 pg-h/mL; and (b) a Cₘₐₓ of tiotropium of 12-19.5 pg/mL.

8. The composition of claim 4, wherein the nebulized dose of the pharmaceutical composition is bioequivalent when compared to: an improvement in trough FEV₁ compared to placebo of the inhaler-dispensed dose of the tiotropium solution.

9. The composition of claim 4, wherein the nebulized dose is nebulized in an air-driven jet nebulizer.

10. The composition of claim 1, wherein the concentration of the sodium citrate is in the range of 0.1-0.11 wt. %.

11. The composition of claim 10, wherein the concentration of citric acid is 0.4 wt. %.

12. The composition of claim 11, wherein the concentration of sodium chloride is 0.8 wt. %.

13. The composition of claim 12, wherein the composition is exclusive of additional excipients.

14. A pharmaceutical product, comprising:
i) a container having a volume in the range of 0.25-6 mL and;
ii) a volume of a benzalkonium chloride-free, EDTA-free complexing agent-free pharmaceutical composition contained in the container, the pharmaceutical composition comprising:
a) tiotropium;
b) 0.08-0.2 wt. % sodium citrate;
c) 0.2-0.6 wt. % citric acid; and
d) at least 97 wt. % water,
the pharmaceutical product administrable by a nebulizer to provide a nebulized dose of the pharmaceutical composition that is bioequivalent to an inhaler-administered dose of a tiotropium solution.

15. The product of claim 14, wherein the volume of the pharmaceutical composition is 2 mL.

16. The product of claim 14, which contains 14-17.6 mcg of tiotropium.

17. A method of treating a patient, comprising administering a plurality of the pharmaceutical product of claim 14 according to a prescribed treatment schedule to achieve an improvement in trough FEV₁ of at least 10% or at least 100 mL above placebo.

18. The method of claim 17, wherein the prescribed treatment schedule extends for at least 48 weeks and the improvement is maintained from after 1 week of the prescribed treatment until the end of the at least 48 weeks.

19. A method of treating a human COPD patient, comprising administering, once daily, a unit dose of a benzalkonium chloride-free, EDTA-free pharmaceutical composition, the composition comprising:
i) 0.0007-0.00088 wt. % tiotropium;
ii) 0.08-0.2 wt. % sodium citrate;
iii) 0.2-0.6 wt. % citric acid;
iv) 0.6-0.9 wt. % sodium chloride; and
v) 97-99.2 wt. % water,
wherein a 2 mL dose of the benzalkonium chloride-free, EDTA-free pharmaceutical composition containing 14-17.6 mcg of tiotropium dispensed through a nebulizer is bioequivalent to a dose of a tiotropium solution containing 5 mcg of tiotropium dispensed through an inhaler, the tiotropium solution comprising water, tiotropium bromide monohydrate, edetate disodium, benzalkonium chloride and hydrochloric acid.

20. The method of claim 19, wherein the nebulized dose of the pharmaceutical composition provides an improvement in trough FEV₁ in a patient that is in the range of 80-125% of an improvement in trough FEV₁ of the inhaler-dispensed dose of the tiotropium solution.
